(19) United States
(12) Patent Application Publication
(10) Pub. No.: US 2023/0080265 A1
(43) Pub. Date: Mar. 16, 2023

(54) HUMANIZED MOUSE MODELS FOR STUDY OF COVID-19

(71) Applicant: Synbal, Inc., San Diego, CA (US)

(72) Inventors: ChenYen Lai, Escondido, CA (US); Oscar Alvarez, San Marcos, CA (US); Kurt Jarnagin, San Mateo, CA (US)

(21) Appl. No.: 17/850,770

(22) Filed: Jun. 27, 2022

Related U.S. Application Data
(60) Provisional application No. 63/237,445, filed on Aug. 26, 2021.

(51) Int. Cl. A01K 67/027; C12N 9/48; C12N 15/85; C12N 9/64

(52) U.S. Cl. CPC A01K 67/0278; C12N 9/485; C12Y 304/17023; C12N 15/8509; C12N 9/6424; C12N 2015/8581; A01K 2207/15; A01K 2227/105; A01K 2267/0337

(57) ABSTRACT

The present disclosure relates to genetically modified non-human animals (e.g., genetically-modified mice or rodents) that express a hACE2 and/or hTMPRRS2 under control of the mouse promoter and the genetically modified non-human animal does not express native ACE2 and/or hTMPRRS2. The present disclosure also relates to methods of generating the genetically-modified animals (e.g., genetically modified mice or rodents), and methods of using the genetically modified non-human animals (e.g., genetically modified mice or rodents) described herein.

### RELATED APPLICATIONS

This application claims priority to and the benefit of U.S. Provisional Patent Application Ser. No. 63/237,445, filed Aug. 26, 2021, and entitled “Humanized Mouse Models for Study of COVID-19”, the disclosure of which is incorporated herein by reference in its entirety.

### GOVERNMENT SPONSORSHIP

This invention was made with government support under grant number 1 R44 A1157900-01A1, awarded by the NIH-NIAID-Small Business Innovation Research (SBIR) program. The government has certain rights in the invention.

### SEQUENCE LISTING

This instant application contains a Sequence Listing which has been filed electronically in ASCII format and is hereby incorporated by reference in its entirety. Said ASCII copy, created on Nov. 23, 2022, is named “131764-0001UT01.txt” and is 88.6 KB bytes in size.

### FIELD

The present application relates to transgenic mouse models and methods for making the same for use in studying viral infections, including those caused by SARS-CoV-2.

### BACKGROUND

The global COVID-19 pandemic has seen more than 4.3 million deaths globally, leading to an unparalleled health and economic crisis worldwide (see, for e.g.: Dong et al. Lancet Infect Dis. 2020; 20(5): 533-41). The disease is caused by a new strain of coronavirus named severe acute respiratory syndrome coronavirus-2 or SARS-CoV-2 (see, for e.g.: Zhou et al. Nature. 2020; 579(7798): 270-3). Many coronaviruses are common human pathogens with low pathogenicity, such as the ones that cause the common cold. A general feature of coronavirus is the spike protein (S), a class I fusion protein that mediates attachment of the virus to cell surface receptors followed by uptake (see, for e.g.: Zhou et al., ibid). Proteolytic cleavage of the S protein by specific proteases and fusion of viral and endosomal membranes triggers the release of viral RNA into the cytosol (see, for e.g.: Fehr and Perlman Methods Mol Biol. 2015; 1282: 1-23). SARS-CoV-2 binds to the extracellular receptor angiotensin-converting enzyme 2 (ACE2) to enter the host's cells, and uses the cellular transmembrane serine protease TMPRSS2 as a cooperating partner for S protein cleavage and priming (see, for e.g.: Hoffmann et al. Cell. 2020. Epub 2020/03/07). FIG. 1, adapted from Heurich et al. Journal of Virology 2014; 88(2): 1293-307, illustrates the mechanism of SARS-COV-2 infection in host cells. Thus, ACE2 and TMPRSS2 are leading targets for antiviral therapies and vaccine development against SARS-CoV-2 (see, for e.g.: Amanat et al. Immunity. 2020; 52(4): 583-9). In addition, there is a need to improve modeling pharmacokinetic behavior of human IgG in mice to assess the activity of human anti-SARS-Cov-2 antibodies as a passive treatment of first responders, at-risk individuals, and other individuals immediately after initial exposure.

Individuals with COVID-19 manifest a wide range of symptoms, ranging from mild disease to fatal illness. Mild COVID-19 with fever, headache, muscle pain, sore throat, or cough may progress to life-threatening illness with dyspnea and cyanosis. Increasing reports indicate that many severe COVID-19 patients also exhibit signs of liver, heart, and kidney damage, diarrhea, conjunctivitis, stroke, seizure, and encephalitis (see, for e.g.: Wadman et al. Science. 2020; 368(6489): 356-60). Thus there is an urgent and critical need for well-defined, genetically tractable small animal models for SARS-CoV-2 infection that capture a wide spectrum of illnesses ranging from mild to lethal COVID-19 disease.

A suitable mouse model is essential for understanding disease pathogenesis and for evaluating the safety and efficacy of vaccine candidates, antibody candidates, or antiviral compounds. However, mice are not infectable with SARS-CoV-2 due to differences between the human and murine ACE2 (see, for e.g.: Zhou et al. Nature. 2020; 579(7798): 270-3). A few transgenic mouse models expressing the human ACE2 gene are available (see, for e.g.: McCray et al. J Virol. 2007; 81(2): 813-21) but show limited infectability by SARS-CoV-2 and develop mild or overly strong symptoms upon infection (see, for e.g.: Bao et al. The Pathogenicity of SARS-CoV-2 in hACE2 Transgenic Mice. bioRxiv. 2020: 2020.02.07.939389). The weaknesses of the current humanized models have been highlighted in scientific media and call for new models that reproduce key features of the human disease (see, for e.g.: Callaway, E. Nature. 2020; 579(7798): 183). The existing mouse models were created using first-generation transgenic approaches and have significant limitations. First, these mice still express the mouse ACE2 receptor, which can confound data from treatments aimed at disrupting the binding of ACE2 to the virus using small molecules or antibodies. Second, they do not express the human ACE2 gene in the same cellular pattern as the endogenous protein (see, for e.g.: McCray et al. J Virol. 2007; 81(2): 813-21). This is critical, as a wide variety of cell types, including pneumocytes, cardiomyocytes, cardiac fibroblasts, and coronary endothelial cells express high levels of ACE2 and are susceptible to infection with SARS-CoV-2 and may contribute to severe disease (see, for e.g.: Chen et al. Cardiovasc Res. 2020; 116(6): 1097-100). Also, it is likely that the humanization of a single receptor is not sufficient to achieve full infectivity in mice, as evidenced by the mild lung pathology in existing humanized models; thus, the human TMPRSS2 may be needed for efficient infection by SARS-CoV-2 (see, for e.g.: Hoffmann et al. Cell. 2020. Epub 2020/03/07). Lastly, all existing humanized models have been constructed in the Th1-dominant mouse strain C57BL/6; this strain exhibits mild symptoms upon viral and bacterial infections (see, for e.g.: Chen et al. J Virol. 2010; 84(3): 1289-301) and is generally considered a poor strain for respiratory investigations; indeed, Th2-dominant BALB/c mice appear to be better models of COVID-19 than C57BL/6 (see, for e.g.: Dinnon et al. Nature 2020. Epub 2020/08/28). Therefore, existing humanized mouse strains poorly model the wide range of disease severity observed in humans and are not suitable for COVID-19 research.

### SUMMARY

This Summary is provided to introduce a selection of concepts in a simplified form that are further described below in the Detailed Description. This Summary is not intended to identify key aspects or essential aspects of the claimed subject matter.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

As embodied and broadly described herein, an aspect of the present disclosure relates to a construct comprising a human ACE2 cDNA minigene comprising a start codon, and an SV40 mini-intron comprising a splice donor site and a splice acceptor site within the cDNA sequence of human ACE2. In various embodiments, the construct comprises a sequence having at least 80% sequence homology with the sequence of “pSYNB026_57BL6_Ace2_Humanizing Construct” (SEQ ID NO: 1), or any fragment thereof. In various embodiments, the construct comprises a sequence having at least 80% sequence homology with the sequence of “pSYNB028_Ace2_NEO_BalbC” (SEQ ID NO: 2), or any fragment thereof. In various embodiments, the construct further comprises a mouse promotor sequence for the mouse ACE2 gene.

In another aspect, described herein is a genetically-modified mouse comprising in its genome a human ACE2 cDNA minigene comprising a start codon, and an SV40 mini-intron comprising a splice donor site and a splice acceptor site within the cDNA sequence of human ACE2, and a start codon of native mouse ACE2, wherein the start codon of the human ACE2 cDNA minigene is upstream of the start codon of native mouse ACE2, and wherein the human ACE2 cDNA minigene disrupts expression of native mouse ACE2. In various embodiments, no more than 20 amino acids are encoded between the start codon of the human ACE2 cDNA minigene and the start codon of native mouse ACE2. In various embodiments, the mouse is a C57BL/6 mouse. In various embodiments, the mouse is a BALB/c mouse. In various embodiments, the human cDNA minigene is under control of a promotor sequence for the native mouse ACE2 gene.

In another aspect, described herein is a construct comprising a human TMPRSS2 cDNA minigene, the human TMPRSS2 cDNA minigene comprising a start codon, and an SV40 mini-intron comprising a splice donor site and a splice acceptor site within the cDNA sequence of human TMPRSS2. In various embodiments, the construct comprises a sequence having at least 80% sequence homology with the sequence of “pSYNB027_C57BL6_Tmprss2_Humanizing Construct” (SEQ ID NO: 3), or any fragment thereof. In various embodiments, the construct comprises a sequence having at least 80% sequence homology with the sequence of “pSYNB029_BalbC_Tmprss2_NEO Humanizing Construct” (SEQ ID NO: 4), or any fragment thereof. In various embodiments, the construct further comprises a mouse promotor sequence for the mouse TMPRSS2 gene.

In another aspect, described herein is a genetically-modified mouse comprising a human TMPRSS2 cDNA minigene, the human TMPRSS2 cDNA minigene comprising a start codon, and an SV40 mini-intron comprising a splice donor site and a splice acceptor site within the cDNA sequence of human TMPRSS2, the mouse further comprising in its genome a start codon for native mouse TMPRSS2, wherein the start codon of the human TMPRSS2 cDNA minigene is upstream of the start codon for native mouse TMPRSS2, and wherein the human TMPRSS2 cDNA minigene disrupts expression of native mouse TMPRSS2. In various embodiments, no more than 20 amino acids are encoded between the start codon of the human TMPRSS2 cDNA minigene and the start codon of native mouse TMPRSS2. In various embodiments, the mouse is a C57BL/6 mouse. In various embodiments, the mouse is a BALB/c mouse. In various embodiments, the mouse further comprises in its genome a human ACE2 cDNA minigene, the human ACE2 cDNA minigene comprising a start codon, and an SV40 mini-intron comprising a splice donor site and a splice acceptor site within the cDNA sequence of the human ACE2 cDNA minigene. In various embodiments, the start codon of the human ACE2 cDNA minigene is upstream of the start codon for native mouse ACE2, and the human ACE2 cDNA minigene disrupts expression of native mouse ACE2. In various embodiments, the human TMPRSS2 cDNA minigene is under control of the promotor sequence for the native mouse TMPRSS2 gene.

In another aspect, described herein is a genetically-modified mouse comprising in its genome at least one chromosome comprising a nucleic acid sequence encoding a human cDNA ACE2 minigene, wherein the mouse does not express endogenous ACE2. In various embodiments, the nucleic acid sequence encoding the human ACE2 is operably linked to an endogenous regulatory element at the endogenous ACE2 gene locus in the at least one chromosome.

In another aspect, described herein is a genetically-modified mouse comprising it its genome at least one chromosome comprising a nucleic acid sequence encoding a human cDNA TMPRSS2 minigene, wherein the mouse does not express endogenous TMPRSS2. In various embodiments, the nucleic acid sequence encoding the human TMPRSS2 is operably linked to an endogenous regulatory element at the endogenous TMPRSS2 gene locus in the at least one chromosome.

In another aspect, described herein is a method of making a genetically-modified mouse, the method comprising replacing in at least one cell of the animal, at an endogenous ACE2 gene locus, a sequence encoding a region of an endogenous ACE2 with a sequence encoding a corresponding region of human cDNA ACE2 minigene. In various embodiments, the mouse comprises a start codon of the human ACE2 cDNA minigene and a start codon of native mouse ACE2 gene, and wherein the start codon of the human ACE2 cDNA minigene is less than 20 amino acids from the start codon of native mouse ACE2 gene. In various embodiments, the mouse expresses the human cDNA ACE2 minigene.

### BRIEF DESCRIPTION OF THE FIGURES

In the Present Application:

FIG. 1 illustrates the mechanism of SARS-CoV-2 infection of host cells, and mouse models generated in accordance with embodiments of the disclosure.

FIG. 2 illustrates the general strategy for mouse humanization in accordance with embodiments of the disclosure.

FIG. 3 illustrates a C57BL6-ACE2 humanizing construct in accordance with embodiments of the disclosure.

FIGS. 4A and 4B illustrate PCR primer targets in C57BL6-ACE2 founder genotype assays in accordance with embodiments of the disclosure.

FIG. 5 shows PCR genotyping data for C45BL6-ACE2 founders in accordance with embodiments of the disclosure.

FIG. 6 illustrates an ACE2 knock-in allele validation assay in accordance with embodiments of the disclosure.

FIG. 7 illustrates off-target analysis of ACE2 animals on C57BL/6 background in accordance with embodiments of the disclosure.

FIG. 8 illustrates a C57BL6-TMPRSS2 humanizing construct in accordance with embodiments of the disclosure.

FIG. 9 illustrates PCR primer targets in C57BL6-TMPRSS2 founder genotype assays in accordance with embodiments of the disclosure.

FIG. 10 shows TMPRSS2-B6 founder genotyping data in accordance with embodiments of the disclosure.

FIG. 11 illustrates a summary of hTMPRSS2-B6 founder genotyping data in accordance with embodiments of the disclosure.

FIG. 12 illustrates a TMPRSS2 knock-in allele validation assay in accordance with embodiments of the disclosure.

FIG. 13 illustrates a BalbC-ACE2 humanizing construct in accordance with embodiments of the disclosure.

FIGS. 14A and 14B illustrate hACE2-NEO ES cell screening assays in accordance with embodiments of the disclosure.

FIGS. 15A and 15B show hACE2 knock-in Southern blot validation assay design and results for hACE2 knock-in ES cells in accordance with embodiments of the disclosure.

FIG. 16 illustrates sequence analysis of hACE2 knock-in allele from animal ACE2-C B4.13 in accordance with embodiments of the disclosure.

FIGS. 17A and 17B illustrate ACE2 and TMPRSS2 off-target sites and primer sequences in accordance with embodiments of the disclosure.

FIG. 18 illustrates off-target analysis of ACE2 animals on BALB/c background in accordance with embodiments of the disclosure.

FIG. 19 illustrates a BalbC-Tmprss2 humanizing construct in accordance with embodiments of the disclosure.

FIGS. 20A-20C show hTMPRSS2 Southern blot validation assay design and results for hTMPRSS2 knock-in ES cells in accordance with embodiments of the disclosure.

FIG. 21 illustrates a TMPRSS2 knock-in allele validation assay in accordance with embodiments of the disclosure.

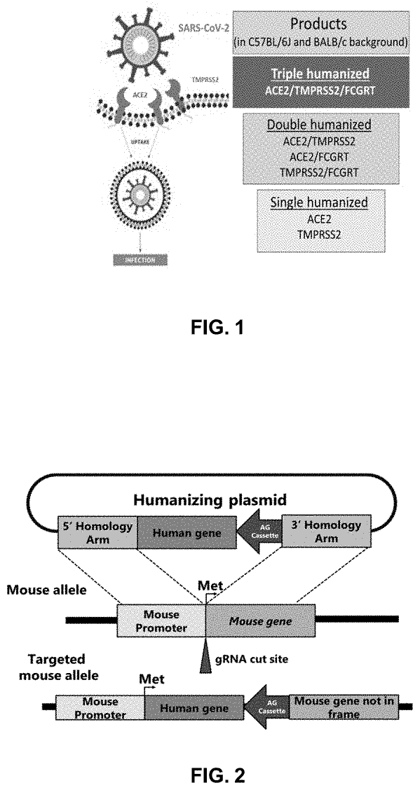

### DETAILED DESCRIPTION

A detailed description of one or more embodiments of the invention is provided below along with accompanying figures that illustrate the principles of the invention. The invention is described in connection with such embodiments, but the invention is not limited to any embodiment. The scope of the invention is limited only by the claims. Numerous specific details are set forth in the following description in order to provide a thorough understanding of the invention. These details are provided for the purpose of non-limiting examples and the invention may be practiced according to the claims without some or all of these specific details. For the purpose of clarity, technical material that is known in the technical fields related to the invention has not been described in detail so that the invention is not unnecessarily obscured.

### Overview of the Present Disclosure

The present disclosure describes nucleic acid constructs and methods for generating rodent models expressing human ACE2 and/or TMPRSS2, and not expressing native ACE2 and/or TMPRSS2.

### Definitions and Interpretation

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

As may be used herein, the term “animal” refers to living multi-cellular vertebrate organisms, a category that includes, for example, mammals and birds. The term “mammal” includes both human and non-human mammals, e.g., bovines, canines, felines, rodents such as rat, murines, simians, equines and humans. Additional examples include adults, juveniles and infants.

The terms “subject,” “host,” “individual,” and “patient” may be used interchangeably herein to refer to animals, typically mammalian animals. Any suitable mammal can be treated by a method, cell or composition described herein. A mammal is a human. A mammal may be any age or at any stage of development (e.g., an adult, teen, child, infant, or a mammal in utero). A mammal may be male or female. A mammal may be a pregnant female. In some embodiments a subject may be a human. In some embodiments, a subject may have, has, may be suspected, or is suspected of having a cancer or neoplastic disorder.

As may be used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “vector” refers to a nucleic acid construct deigned for transfer between different hosts, including but not limited to a plasmid, a virus, a cosmid, a phage, a BAC, a YAC, etc. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.). The term “construct” refers to a recombinant nucleic acid that has been generated for the purpose of the expression of a specific nucleotide sequence.

It is to be inferred without explicit recitation and unless otherwise intended, that when the present disclosure relates to a polypeptide, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide or nucleic acid, intends those having minimal homology while still maintaining desired structure or functionality. Unless specifically recited herein, it is contemplated that any of the above also includes equivalents thereof. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and/or exhibits substantially equivalent biological activity to the reference protein, polypeptide, or nucleic acid. Alternatively, when referring to polynucleotides, an equivalent thereof is a polynucleotide that hybridizes under stringent conditions to the reference polynucleotide or its complement. Specifically, in some embodiments, the sequence encoding a human ACE2 comprises a sequence encoding an amino acid sequence that is at least 50%, 55%, 65%, 70%, 75%, 80%, 85%, 90%, 95%, 99%, or 100% identical to human ACE2 (pSYNB026_C57BL6_Ace2_Humanizing Construct; SEQ ID NO: 1). Specifically, in some embodiments, the sequence encoding a human ACE2 comprises a sequence encoding an amino acid sequence that is at least 50%, 55%, 65%, 70%, 75%, 80%, 85%, 90%, 95%, 99%, or 100% identical to human ACE2 (pSYNB028_Ace2_NEO_BalbC; SEQ ID NO: 2). Specifically, in some embodiments, the sequence encoding a human TMPRSS2 comprises a sequence encoding an amino acid sequence that is at least 50%, 55%, 65%, 70%, 75%, 80%, 85%, 90%, 95%, 99%, or 100% identical to human TMPRSS2 (pSYNB027_C57BL6_Tmprss2_Humanizing Construct; SEQ ID NO: 3). Specifically, in some embodiments, the sequence encoding a human TMPRSS2 comprises a sequence encoding an amino acid sequence that is at least 50%, 55%, 65%, 70%, 75%, 80%, 85%, 90%, 95%, 99%, or 100% identical to human TMPRSS2 (pSYNB029_BalbC_Tmprss2_NEO Humanizing Construct; SEQ ID NO: 4). Stated another way, in one aspect, the disclosure relates to mouse and rodents comprising at least one cell comprising a nucleotide sequence encoding a ACE2 gene wherein the ACE2 gene comprises at least 50 contiguous amino acid residues that are identical to the corresponding contiguous amino acid sequence of a human ACE2 gene, wherein the animal expresses the human ACE2 gene. Stated another way, in one aspect, the disclosure relates to mouse and rodents comprising at least one cell comprising a nucleotide sequence encoding a TMPRRS2 gene wherein the TMPRRS2 gene comprises at least 50 contiguous amino acid residues that are identical to the corresponding contiguous amino acid sequence of a human TMPRRS2 gene, wherein the animal expresses the human TMPRRS2 gene.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

As may be used herein, the term “recombinant,” when used to modify “protein,” “peptide,” or “polypeptide,” or any specific protein, peptide, or polypeptide, refers to a protein, peptide, or polypeptide produced by recombinant DNA techniques, wherein generally, DNA encoding the polypeptide is inserted into a suitable expression vector, which is in turn used to transform a host cell to produce the heterologous protein.

As used herein, the term “transgenic” refers to an organism that contains genetic material into which DNA from an unrelated organism has been artificially introduced. As used herein, the term “transgene” refers to a gene that has been transferred from one organism to another.

As used herein, the term “genome editing” refers to insertion, deletion, modification, or replacement of a genome of an organism. The terms “knock-in” and “knock-out”, as used herein, refer to the addition of a gene sequence into a genome and the inactivation or removal of a gene, respectively.

As used herein, the term “allele” refers to one of two or more versions of a gene. “Homozygous” refers to an animal having two alleles for a gene, where both alleles are the same. “Heterozygous” refers to an animal having two alleles for a gene, where the two alleles are different.

As used herein, the term “wild type” refers to a phenotype, a genotype, or gene that predominates in a natural population of organisms or strain of organisms.

As used herein, the term “endogenous” or “native” refers to a gene that originates from within an organism.

As used herein, the term “cassette” or “gene cassette” is a fragment of DNA carrying, and capable of expressing, one or more genes or interest between one or more sets of restriction sites.

As used herein, the term “locus” refers to a specific, fixed position on a chromosome where a particular gene or genetic marker is located.

A “humanized mouse” refers to a mouse carrying functioning human genes, cells, tissues, and/or organs. As used herein a “founder” mouse refers to a mouse which has integrated the transgenic construct.

The term “isolated” as may be used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double and single stranded molecules. Unless otherwise specified or required, any aspect of this technology that is a polynucleotide encompasses both the double stranded form and each of two complementary single stranded forms known or predicted to make up the double stranded form.

As may be used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

“Immune cells” includes, e.g., white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells). A “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Cytokines are small secreted proteins released by immune cells that have a specific effect on the interactions and communications between said immune cells. Cytokines can be pro-inflammatory or anti-inflammatory. A non-limiting example of a cytokine is granulocyte-macrophage colony-stimulating factor (GM-CSF), which stimulates stem cells to produce granulocytes (neutrophils, eosinophils, and basophils) and monocytes.

As used herein, the phrase “immune response” or its equivalent “immunological response” refers to the development of a cell-mediated response (e.g., mediated by antigen-specific T cells or their secretion products). A cellular immune response is elicited by the presentation of polypeptide epitopes in association with Class I or Class II MHC molecules, to treat or prevent a viral infection, expand antigen-specific B-reg cells, TC1, CD4+ T helper cells and/or CD8+ cytotoxic T cells and/or disease generated, autoregulatory T cell and B cell “memory” cells. The response may also involve activation of other components. In some aspect, the term “immune response” may be used to encompass the formation of a regulatory network of immune cells. Thus, the term “regulatory network formation” may refer to an immune response elicited such that an immune cell, preferably a T cell, more preferably a T regulatory cell, triggers further differentiation of other immune cells, such as but not limited to, B cells or antigen-presenting cells—non-limiting examples of which include dendritic cells, monocytes, and macrophages. In certain embodiments, regulatory network formation involves B cells being differentiated into regulatory B cells; in certain embodiments, regulatory network formation involves the formation of tolerogenic antigen-presenting cells.

As may be used herein, the term “antibody” (“Ab”) collectively refers to immunoglobulins (or “Ig”) or immunoglobulin-like molecules including but not limited to antibodies of the following isotypes: IgM, IgA, IgD, IgE, IgG, and combinations thereof. Immunoglobulin-like molecules include but are not limited to similar molecules produced during an immune response in a vertebrate, for example, in mammals such as humans, rats, goats, rabbits and mice. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also Pierce Catalog and Handbook, 1994-1995 (Pierce Chemical Co., Rockford, Ill.); Kuby, Immunology, 3rd Ed., W. H. Freeman & Co., New York, 1997.

As may be used herein, the term “monoclonal antibody” refers to an antibody produced by a cell into which the light and heavy chain genes of a single antibody have been transfected or, more traditionally, by a single clone of B-lymphocytes. Monoclonal antibodies generally have affinity for a single epitope (i.e., they are monovalent) but may be engineered to be specific for two or more epitopes (e.g., bispecific). Methods of producing monoclonal antibodies are known to those of skill in the art, for example by creating a hybridoma through fusion of myeloma cells with immune spleen cells, phage display, single cell amplification from B-cell populations, single plasma cell interrogation technologies, and single B-cell culture. Monoclonal antibodies include recombinant antibodies, chimeric antibodies, humanized antibodies, and human antibodies.

As may be used herein, the term “antigen” refers to a compound, composition, or substance that may be specifically bound and/or recognized by the products of specific humoral or cellular immunity and antigen recognition molecules, including but not limited to an antibody molecule, single-chain variable fragment (scFv), cell surface immunoglobulin receptor, B-cell receptor (BCR), T-cell receptor (TCR), engineered TCR, modified TCR, or CAR. The term “epitope” refers to an antigen or a fragment, region, site, or domain of an antigen that is recognized by an antigen recognition molecule. Antigens can be any type of molecule including but not limited to peptides, proteins, lipids, phospholipids haptens, simple intermediary metabolites, sugars (e.g., monosaccharides or oligosaccharides), hormones, and macromolecules such as complex carbo-hydrates (e.g., polysaccharides). Some non-limiting examples of antigens include antigens involved in autoimmune disease (including autoantigens), allergy, and graft rejection, tumor antigens, toxins, and other miscellaneous antigens. Non-limiting examples of tumor antigens include mesothelin, ROR1 and EGFRvIII, ephrin type-A receptor 2 (EphA2), interleukin (IL)-13r alpha 2, an EGFR VIII, a PSMA, an EpCAM, a GD3, a fucosyl GM1, a PSCA, a PLAC1, a sarcoma breakpoint, a Wilms Tumor 1, a hematologic differentiation antigen, a surface glycoprotein, a gangliosides (GM2), a growth factor receptor, a stromal antigen, a vascular antigen, or a combination thereof. Antigens expressed by pathogens include, but are not limited to, microbial antigens such as viral antigens, bacterial antigens, fungal antigens, protozoa, and other parasitic antigens.

As may be used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding the chimeric PVX described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical,” percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

As used herein, “treating” or “treatment” of a disease in a subject refers to: (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

As used herein, the term “vaccine” refers to a biological preparation that provides active acquired immunity to a particular infectious disease. As used herein, the term “antiviral therapy” refers to a treatment with a drug(s) that inhibit the ability of a virus(es) to multiply in the body of a subject.

As may be used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As may be used herein, the term “introduce” as applied to methods of producing modified cells refers to the process whereby a foreign (i.e., extrinsic or extracellular) agent is introduced into a host cell thereby producing a cell comprising the foreign agent. Methods of introducing nucleic acids include but are not limited to transduction, retroviral gene transfer, transfection, electroporation, transformation, viral infection, microinjection, and other recombinant DNA techniques known in the art. Transduction may be done via a vector (e.g., a viral vector). Transfection may be done via a chemical carrier, DNA/liposome complex, or micelle (e.g., Lipofectamine (Invitrogen)) or microinjection, ballistic-assisted transfection, electric-assisted transfection, or sonic-assisted transfection. Viral infection may be done via infecting the cells with a viral particle comprising the polynucleotide of interest (e.g., AAV). Introduction may comprise CRISPR mediated gene editing or Transcription activator-like effector nuclease (TALEN) mediated gene editing. Methods of introducing non-nucleic acid foreign agents (e.g., soluble factors, cytokines, proteins, peptides, enzymes, growth factors, signaling molecules, small molecule inhibitors) include but are not limited to culturing the cells in the presence of the foreign agent, contacting the cells with the agent, contacting the cells with a composition comprising the agent and an excipient, and contacting the cells with vesicles or viral particles comprising the agent.

In the context of a nucleic acid or amino acid sequence, the term “chimeric” intends that the sequence contains is comprised of at least one substituent unit (e.g., fragment, region, portion, domain, polynucleotide, or polypeptide) that is derived from, obtained or isolated from, or based upon other distinct physical or chemical entities. For example, a chimera of two or more different proteins may comprise the sequence of a variable region domain from an antibody fused to the transmembrane domain of a cell signaling molecule. In some aspects, a chimera intends that the sequence is comprised of sequences from at least two distinct species.

Other examples of implementations will become apparent to the person skilled in the art in view of the teachings of the present description and as such, will not be further described here.

Note that titles or subtitles may be used throughout the present disclosure for convenience of a reader, but in no way should these limit the scope of aspects or embodiments of the invention(s) detailed herein. Moreover, certain theories may be proposed and disclosed herein; however, in no way they, whether they are right or wrong, should limit the scope of the invention so long as the invention is practiced according to the present disclosure without regard for any particular theory or scheme of action.

Any and all references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

### Detailed Description of Aspects and Embodiments of the Disclosure

Disclosed herein are mouse models for evaluating COVID-19 disease, and methods for generating them. Existing mouse models of COVID-19 disease have humanized ACE2 and were created using traditional transgenic approaches that have several limitations as follows: 1) multiple copies of a human gene are inserted randomly in the genome; 2) expression of the human ACE2 transgene does not have a normal expression pattern across tissues (see, for e.g.: McCray et al. J Virol. 2007; 81(2): 813-21 and Yang et al. Comp Med. 2007; 57(5): 450-9); and 3) the mouse ACE2 gene is not knocked out, potentially leading to confounding effects when testing therapeutics. The approaches described herein are innovative from several perspectives. While, exemplified and described herein, aspects and embodiments of the invention(s) are not limited to mouse models. The methods and principles described herein may be applied to any rodent, including but not limited to, rat, hamster, guinea pig, etc.

First, the models disclosed herein address the limitations in the previous models by using an innovative CRISPR/Cas9 assisted Homology Directed Repair (HDR) technology to simultaneously knock out the endogenous mouse gene while inserting a human cDNA minigene counterpart in the mouse genome under control of the mouse promoter. The general mouse humanization strategy is illustrated in FIG. 2. As shown in FIG. 2, a human cDNA-mini-gene cassette is integrated at, or very near, the methionine translational start site (Met) of the target gene, e.g., no more than 20, 15, 10, 5, 2, etc. amino acids away from the Met of the target gene. A targeting vector containing the human gene sequence, homology arms, and an Active Genetics cassette is inserted using CRISPR-Cas9 into the mouse genome. This results in the minigene under the control of the mouse target gene promoter, with natural tissue/cell expression patterns. This approach includes several elements to ensure optimal expression levels (an SV40 intron that increases gene expression levels, 3′UTR sequence of the human gene to improve the export, stability, decay, and mRNA translation and to prevent abnormal protein expression due to cryptic promoters or aberrant splicing, 3′ splicing acceptor followed by stop sequences in each of the three reading frames).

Second, humanized mice were generated on two different genetic backgrounds: C57BL/6 with a predominantly Th1 response, and BALB/c with a Th2-dominant response. This approach, in combination with the generation of singly and doubly humanized ACE2/TMPRSS2 mice, allowed for the modeling of a wide spectrum of illnesses caused by SARS-CoV-2 infection, ranging from mild to severe COVID-19 disease and different sequelae.

The mouse models provided herein represent a critical tool that allows for testing the safety and efficacy of SARS-CoV-2 antibodies in a human-like setting by addressing practical concerns. There is a concern with vaccines and antibodies that there may be an unwanted antibody-dependent enhancement (ADE) of disease caused by antibody Fc coupling to virus via Fc receptors (FcRs) on immune cells and which facilitates virus uptake and replication. This unwanted action is better modeled in the humanized FcRn mice proposed. Additionally, the clinical success of an antibody-based therapeutic against SARS-CoV-2 is tied to longevity, half-life, which is related to its affinity for the FcRn (encoded by FCGRT) that is expressed on endothelial cell membranes and constantly endocytoses IgG from the plasma and recycles it back to the plasma. Mouse FcRn has a different affinity for human IgG than human FcRn, and transgenic mice expressing human FcRn, but not wild type mice, exhibit antibody PK profiles highly correlated with humans and non-human primates (see, for e.g.: Avery et al. MAbs 2018; 10(2): 244-55, and Tam et al. MAbs 2013; 5(3): 397-405). A humanized FCGRT strain, therefore, provides an accurate modeling of antibody half-lives, virus neutralization, and likelihood of therapeutic antibody-FcRn interactions and immune complex formation. Thus, it is contemplated that the singly and doubly humanized ACE2/TMPRSS2 mouse models provided herein may be triply humanized with human FCGRT for further testing of SARS-CoV-2 antibodies. While the description and examples provided herein include generation of transgenic mice in C57BL/6 and BALB/c backgrounds, other mice and rodents of any background can be used. The invention also contemplates the combination of hACE2 and hTMPRSS2 strains developed herein with other genetically engineered mouse strains, such as strains containing knockouts of other mouse genes, strains containing other humanized loci, or strains containing other transgenes.

As BALB/c mice can develop severe SARS-like disease, it is expected that one or more humanized mice in the BALB/c background serve as models for severe COVID-19, whereas the humanized C57BL/6 mice are likely to serve as models of asymptomatic SARS-CoV-2 infection or mild COVID-19. Mouse models that reproduce a spectrum of SARS-CoV-2-induced illnesses are critical tools for deciphering mechanisms of SARS-CoV-2 pathogenesis and immunity and for developing and testing vaccines and treatments for COVID-19. A thorough comparison of SARS-CoV-2 infection in different humanized mouse strains will identify the most relevant models of the human disease and define key endpoints for evaluation of antiviral and vaccine candidates. Additionally, results obtained from our studies with single hACE2 vs single hTMPRSS2 vs double hACE2/hTMPRSS2 mice may inform the precise role of ACE2 and TMPRSS2 in SARS-CoV-2 infection.

Replacement of non-human genes in a non-human animal with homologous or orthologous human genes or human sequences, at the endogenous non-human locus and under control of the native promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and usually integrates at random into the genome, often in multiple loci. Typically, the location of the integrated transgene is unknown; expression of the human protein is measured by transcription of the human gene and/or protein assay and/or functional assay. Inclusion in the human transgene of upstream and/or downstream human sequences are generally found to be sufficient to provide suitable support for expression and/or regulation of the transgene.

The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal's physiology.

Genetically modified animals that express human or humanized ACE2 or TMPRRS2 gene(s) e.g., in a physiologically appropriate manner, provide a variety of uses that include, but are not limited to, developing therapeutics for human diseases and disorders, and assessing the efficacy of these human therapeutics in the animal models.

In some embodiments, the genetically modified animals can be used for determining effectiveness of vaccines, the development of a new diagnostic strategy and/or a therapeutic strategy. The methods involve administering a vaccine and/or a therapeutic agent to treat SARS-CoV-2 to the animal as described herein; and determining the effects of the vaccine, diagnostic strategy and/or a therapeutic agent. The effects that can be determined include, e.g., a whether the vaccine/diagnostic strategy/therapeutic agent can stimulate an immune response, whether the vaccine/diagnostic strategy/agent can upregulate the immune response or downregulate immune response, decrease of symptoms, decrease the risk of developing complications from infection, increase the survival rate, increase life expectancy, etc. In some embodiments, the genetically modified animals can be used for determining the effective dosage of a therapeutic agent for treating a disease in the subject. The present disclosure also relates to the use of the animal model generated through the methods as described herein in the development of a product related to an immunization processes of human cells, the manufacturing of a human vaccine, or the model system for a research in pharmacology, immunology, microbiology and medicine. The disclosure also relates to the use of the animal model generated through the methods as described herein in the screening, verifying, evaluating or studying the hACE2 or hTMPRRS2 genes function, drugs for human hACE2 or hTMPRRS2 targeting sites, the drugs for immune-related diseases.

In addition, the mice described in the present disclosure can be mated with the mice containing other human or chimeric genes, so as to obtain a mouse expressing two or more human genes. For example, in some embodiments, the mice express hACE2 and hTMPRRS2.

### EXAMPLES

Three genes were humanized; ACE2, TMPRSS2, and FCGRT. ACE2 is the main extracellular receptor that binds the viral S protein and is required for viral entry in target cells. TMPRSS2 is an extracellular serine protease required for S protein priming and subsequent fusion of viral and cellular membranes. FCGRT is a neonatal Fc receptor (also FcRN, IgG receptor FcRN large subunit p51) is the main regulator of IgG pharmacokinetics and is important for testing human antibodies in mice. Triply humanized mouse models (ACE2/TMPRSS2/FCGRT) in C57BL/6J and BALB/c backgrounds were generated, as well as doubly and singly humanized mouse strains. The mouse products generated are shown in Table 1. C57BL/6 and BALB/c mice that are prototypical Th1- and Th2-type mouse strains, respectively. The two backgrounds were used with the goal of observing the range of symptoms in individuals with COVID-19, from mild to fatal illness. It was hypothesized that one or more of the humanized mice in the BALB/c background would model severe COVID-19, whereas the humanized C57BL/6J mice were likely to serve as models of asymptomatic or rapid recovery from infection.

TABLE 1
Humanized Mouse Products Produced

| Product | Strain Background | Strain Name | Humanized Loci |
|---|---|---|---|
| 1 | C57BL/6J | C57BL/6J-ACE2/FCGRT | ACE2, FCGRT |
| 2 | C57BL/6J | C57BL/6J-TMPRSS2/FCGRT | TMPRSS2, FCGRT |
| 3 | C57BL/6J | C57BL/6J-ACE2 | ACE2 |
| 4 | C57BL/6J | C57BL/6J-TMPRSS2 | TMPRSS2 |
| 5 | C57BL/6J | C57BL/6J-ACE2/TMPRSS2 | ACE2, TMPRSS2 |
| 6 | C57BL/6J | C57BL/6J-ACE2/TMPRSS2/FCGRT | ACE2, TMPRSS2, FCGRT |
| 7 | BALB/c | BALB/c-ACE2 | ACE2 |
| 8 | BALB/c | BALB/c-TMPRSS2 | TMPRSS2 |
| 9 | BALB/c | BALB/c-ACE2/TMPRSS2 | ACE2, TMPRSS2 |

### Example 1. Development of Humanized C57BL/6J-ACE2 (ACE2-B6) Knock-In Mouse Strain

The Ace2 locus on the mouse X-chromosome was modified to express the human ACE2 protein. The humanized ACE2 allele was also combined with the humanized FCGRT allele on the C57BL/6J background through breeding.

Guide RNA

The Ace2-crRNA_32 guide RNA was developed and tested. The protospacer sequence is 5′-AAGGCTGAGAAGGAGCCAGG-3′ (SEQ ID NO: 5), targeting Cas9 cleavage 13 bp downstream of the mouse Ace2 start codon in exon 2. The synthetic Ace2-crRNA_32 crRNA was purchased from Integrated DNA Technologies (IDT) and annealed to synthetic tracrRNA (IDT) to produce active guide RNA for genome editing.

Donor Plasmid and Knock-In Allele Design

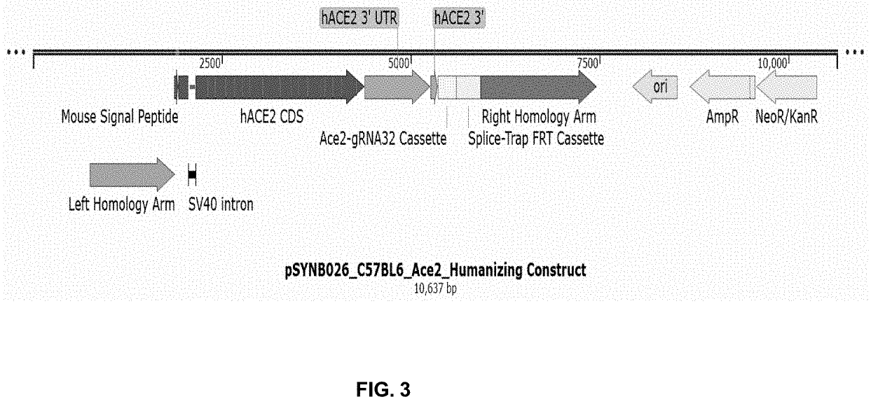

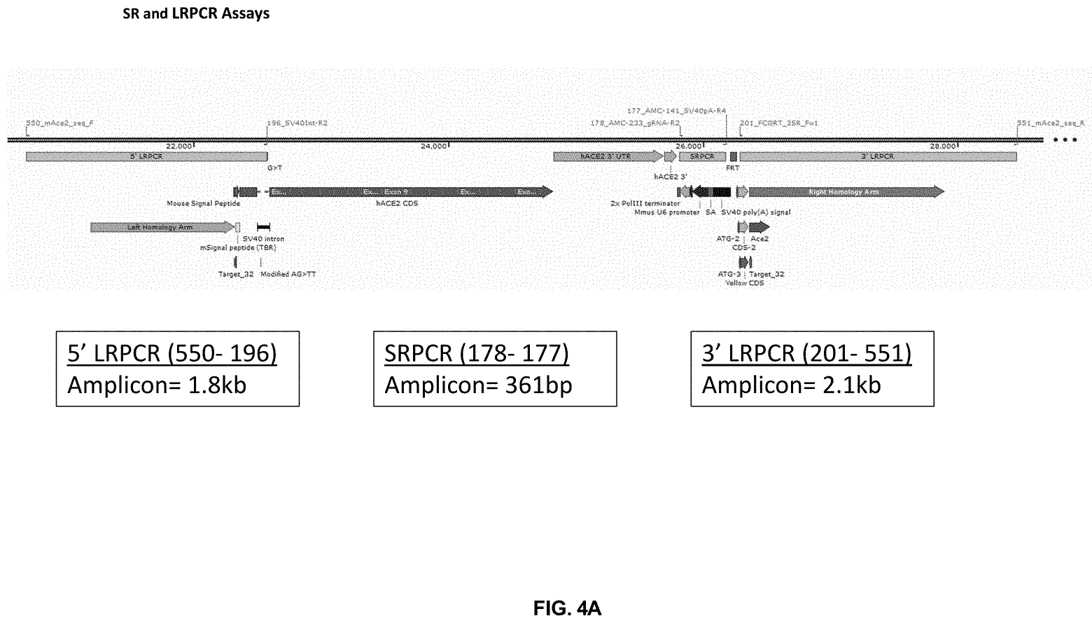

The donor plasmid “pSYNB026_C57BL6_Ace2_Humanizing Construct” (SEQ ID NO: 1) was designed and constructed with a total insert size (region between homology arms) of 4037 bp. The plasmid, shown in FIG. 3, contains the following key features:
1. Left Homology Arm: 1134 bp 5′ homology region encompassing sequences immediately 5′ of the Ace2-crRNA_32 Cas9 cut site
2. Mouse Signal Peptide: signal peptide coding sequence (translation produces amino acid sequence WLLLSLVAVTTA (SEQ ID NO: 6)) in-frame with first 5 amino acids of mouse Ace2
3. hACE2 CDS: human ACE2 cDNA coding sequence with SV40 intron inserted for enhanced expression
4. hACE2 3′UTR: 872 bp human ACE2 3′UTR
5. hACE2 3′: 100 bp human intergenic sequence immediately 3′ of ACE2 polyadenylation region
6. Ace2-gRNA32 Cassette: “Active genetics” cassette comprised of a mouse U6 promoter-driven Ace2-gRNA32 guide RNA with 2×PolIII terminator, all in reverse orientation relative to Ace2
7. Splice-Trap FRT Cassette: splice acceptor and SV40 polyadenylation sequence followed by a Flp Recombinase Target (FRT) site and 3-frame ATG short open reading frames. This cassette is designed to truncate any mouse Ace2 isoforms that may splice past the hACE2 cassette. The FRT site allows Flp-mediated collapse of targeted tandem integration events if required.
8. Right Homology Arm: 1535 bp 3′ homology arm encompassing sequences immediately 3′ of the Ace2-crRNA_32 Cas9 cut site

The donor vector, in glycerol stock, was streaked on an ampicillin plate and a single colony was used to start a maxiprep culture. Maxiprep DNA was analyzed by restriction digestion to confirm the structure and absence of rearrangements prior to microinjection.

### Founder Production Summary

The microinjection session was performed with embryos from C57BL/6J mice as summarized in Table 2. The process consisted of several steps: superovulation and donor embryo harvest, pronuclear microinjection, and reimplantation into pseudopregnant females. At least two microinjections of 100 embryos will be done per DNA construct. The session was performed with enhanced specificity eSpCas9, and yielded 29 pups, with two females having correct integration of the donor vector and 6 additional animals having integration events that scored positive for vector backbone PCRs, suggesting tandem donor integrations.

TABLE 2
hACE2 Knock-in, C57BL/6J Strain, Microinjection Summary

| | | PN injection | | | |
|---|---|---|---|---|---|
| | | (transferred | | | |
| Injection | | embryos −> | Pups | | |
| Round | Cond. | recipients) | Birth Date | Pups | Founder #, Notes |
| 1. | A | 174 1-cell −> 7 | Jul. 28, 2020 | 1 | 1 pup born, missing before toeing |
| 2a. | B | 69 1-cell −> 3 | Aug. 24, 2020 | 0 | |
| 2b. | C | 58 1-cell −> 2 | Aug. 24, 2020 | 0 | |
| 3a. | B | 25 1-cell −> 1 | Aug. 25, 2020 | 0 | |
| | | 30 2-cell −> 1 | | | |
| 3b. | C | 36 2-cell −> 1 | Aug. 25, 2020 | 0 | |
| 4a. | D | 105 1-cell −> 4 | Sep. 30, 2020 | 18 | 15 survived to genotyping; 1F (#2) |
| | | | | | correct integration; 4F, 1M backbone- |
| | | | | | positive (tandem) integrations |
| 4b. | E | 86 1-cell −> 3 | Sep. 30, 2020 | 11 | 1F (#26) correct integration; 1M |
| | | | | | backbone-positive (tandem) integration |

Condition A: 400 nM Cas9, 600 nM crRNA/tracrRNA, 20 ng/μl supercoiled donor, 1-cell C57BL/6J embryos injected.

Founder Genotyping Assays

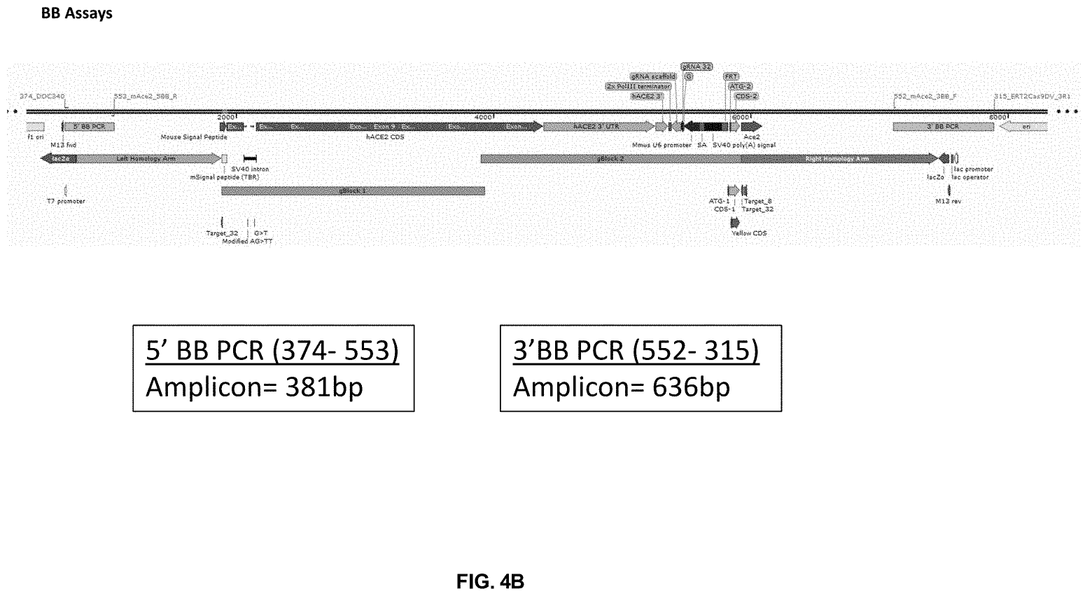

Twenty-six potential founder animals were genotyped with the assays depicted in FIG. 4. The primer sequences are shown in Table 3 below. Short range PCR (SRPCR) assays were performed to detect any founders with donor vector integration. 5′ long range PCR (5′ LRPCR) and 3′ long-range PCR (3′ LRPCR) assays were performed with one primer that anneals in native mouse sequence outside the donor vector homology arm and one primer that anneals in sequence unique to the donor vector. Positive amplification by these primers indicates insertion of the transgene at the target locus.

TABLE 3
Primers and Assay Information

| | Fwd | | Rev | | |
|---|---|---|---|---|---|
| | Primer | | Primer | | Amplicon |
| Assay | Name | Fwd Primer Seq | Name | Rev Primer Seq | Size |
| 5′LRPCR | SB550 | GGTCTCACAAGAGACAGCTATAGAG | SB196 | TGAGGAGCAGTTCTTTGATTTG | 1.8 kB |
| | | (SEQ ID NO: 7) | | (SEQ ID NO: 8) | |
| 3′LRPCR | SB466 | TGCACCATGGTATCACCATGCAC | SB551 | CAGCTGATCATTCATACTCTCCC | 2.2 kB |
| | | (SEQ ID NO: 9) | | (SEQ ID NO: 10) | |
| SRPCR | SB175 | CAAGATGGATTGCACGCAGG | SB177 | AAACCACAACTAGAATGCAGTGA | 361 bp |
| | | (SEQ ID NO: 11) | | (SEQ ID NO: 12) | |
| 5′BB | SB319 | CCGTAAAGCACTAAATCGGAACC | SB553 | AAGATCTCTGAGTTTGAGGACAGC | 685 bp |
| | | (SEQ ID NO: 13) | | (SEQ ID NO: 14) | |
| 3′BB | SB552 | AGCTGAATGGTTCAAGGTGAG | SB315 | CTTTTGCTCACATGTTCTTTCCT | 363 bp |
| | | (SEQ ID NO: 15) | | (SEQ ID NO: 16) | |

5′ and 3′ vector backbone (BB) assays were performed using one primer targeting the homology arm and a second primer targeting the donor vector backbone sequence. BB assays identify random donor integration at off-target sites in the genome as well as tandem integration of multiple donor copies at the target site. Correct targeted homologous integration events exclude the donor vector backbone sequences.

Founder Genotyping Data

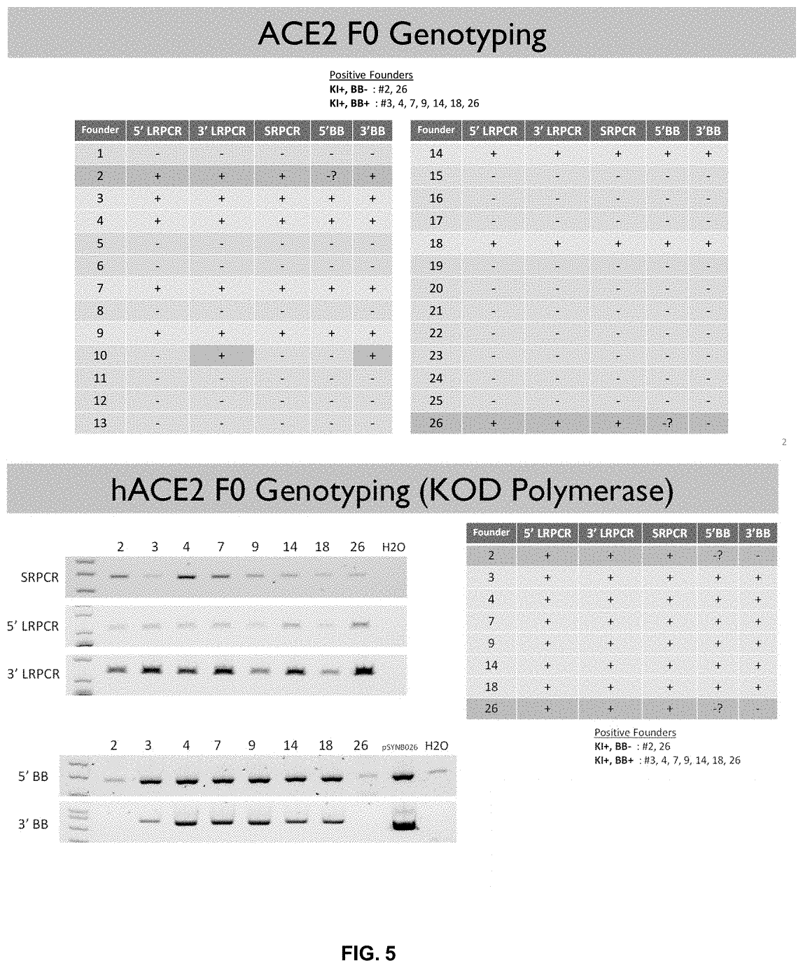

FIG. 5 shows PCR genotyping of potential founder animals 1-26. Tail biopsies were obtained, and founder mice were identified. Animals 2 and 26 were positive for internal short-range assay and 5′ and 3′ long-range assays, and negative for vector backbone assays. Animals 3, 4, 7, 9, 14 and 18 were positive for all assays, suggesting tandem integration at the target locus.

Founder Breeding

Two vector backbone-negative female founders (#2, #26) were mated to males homozygous for the FCGRT humanized knock-in allele (Fcgrt KI/KI). Both females transmitted the hACE2 knock-in event to offspring. However, founder #26 only gave one viable litter while founder #2 produced multiple litters with positive offspring. Offspring from founder #2 were used for subsequent breeding and characterization.

Knock-in Allele Validation

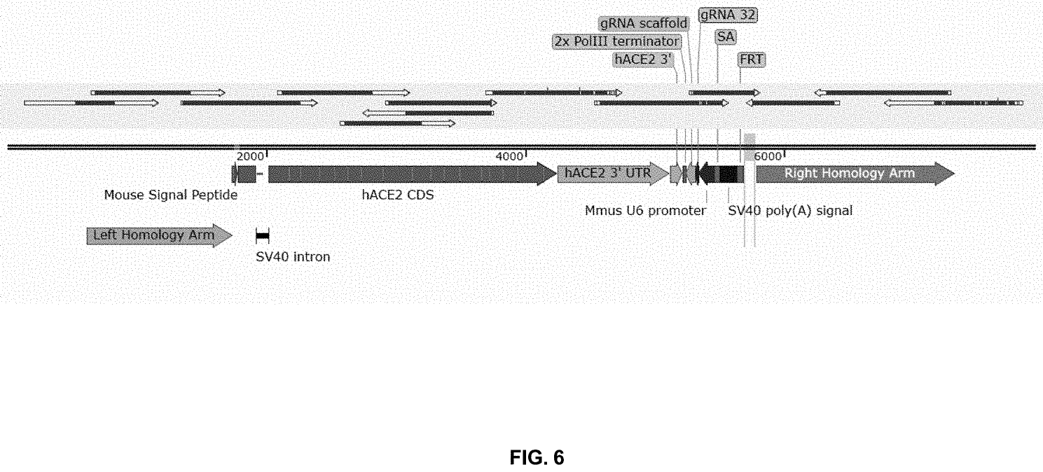

DNA samples from F1 animals were assessed for sequence confirmation of the knock-in allele in founder line 2. Samples were assessed from females ACE2-B6 2.4 and ACE2-B6 2.12 (genotype Ace2 KI/+; Fcgrt KI/+) and males ACE2-B6 2.5 and ACE2-B6 2.14 (genotype Ace2 KI/Y; Fcgrt KI/+). The knock-in allele was PCR amplified and amplicons were sequenced. FIG. 6 shows the sequence analysis of the hACE2 Knock-In Allele from Animal ACE2-B6 2.12. Bars labeled “PCR1,” “PCR2,” PCR3,” and “PCR4” show PCR amplicons produced and Sanger sequenced from animal 2.12. The small arrows at the top of the schematic indicate sequence traces matching expected sequence.

Off-Target Mutation Analysis

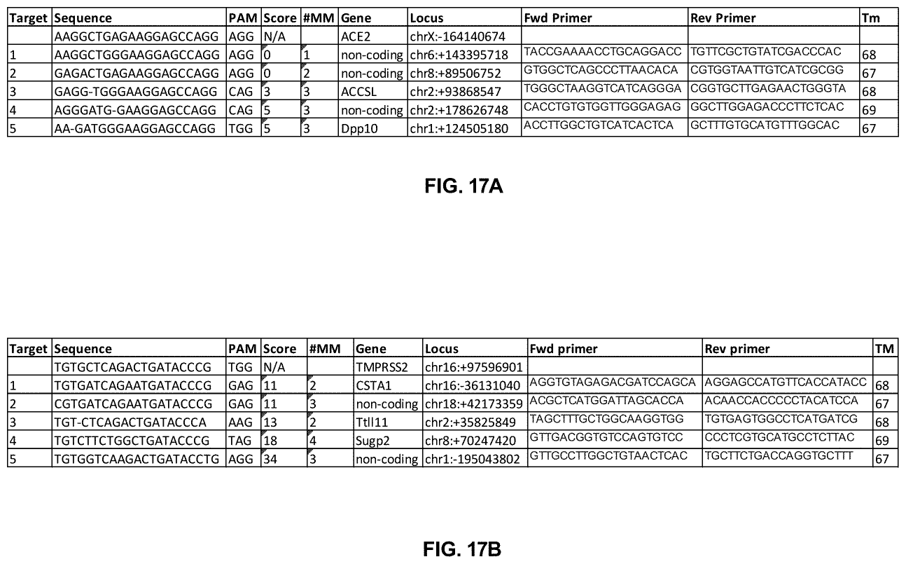

The IDT CRISPR-Cas9 Design Checker online tool was used to identify the top five potential off-target cut sites in the mouse genome. Primers were designed to amplify the target regions for sequencing. The off-target sites and primer sequences are shown in FIG. 17A.

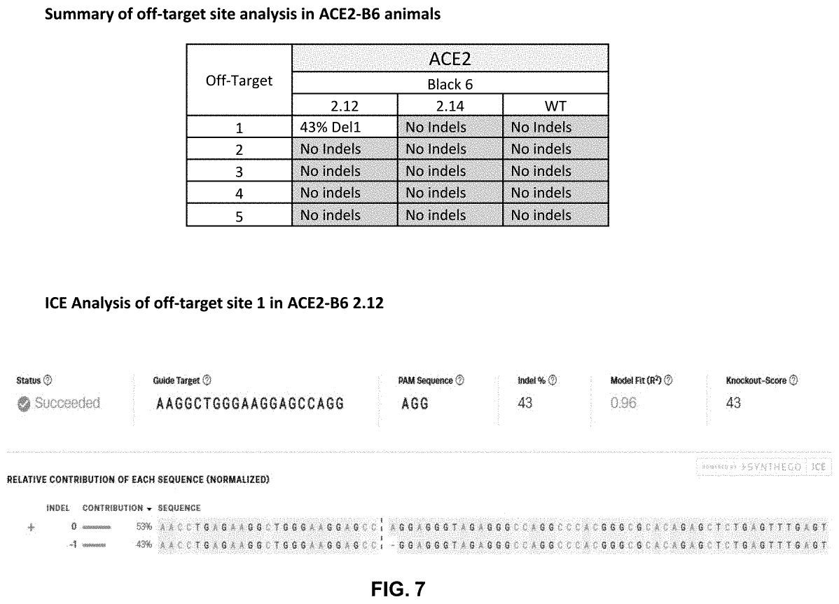

DNA samples from animals ACE2-B6 2.12 and ACE2-B6 2.14 were used for PCR amplification and sequencing across potential off-target sites. Wild-type control animals were included in the analysis. As shown in FIG. 7, ACE2-B6 animal 2.12 was heterozygous for a 1 bp deletion at off-target site #1, while animal 2.14 showed wild-type sequence at the site. Off-target site 1 is a non-coding region on chromosome 6, and mutations at the site are not expected to have any phenotypic consequence.

Colony Expansion Breeding

F1 animals harboring the ACE2 and FCGRT knock-in alleles were intercrossed to generate ACE2 homozygous (KI/KI) females and hemizygous (KI/Y) males with and without the FCGRT knock-in allele. Tissue samples were harvested from animal ACE2-B6 2.46 (Ace2 KI/Y; Fcgrt KI/+) according to the following procedure.

Expression Analysis

Animals are euthanized by CO2 inhalation followed by cervical dislocation.

Tissues pieces are placed immediately in 500 μl RNAlater (aqueous solution) at room temperature, then moved to 4° C. Tissues in RNAlater are held at 4° C. at least overnight, and frozen at −80° C. until ready for analysis.

Tissue Samples for Harvesting
1. Intestine
2. Kidney
3. Prostate
2. Lung divided into 2 pieces ˜100 mg each.
6. Heart collected and divided into 2×˜75 mg pieces.
Transgene expression is assessed.

### Example 2. Development of Humanized C57BL/6J-TMPRSS2 Knock-In Mouse Strain

The Tmprss2 locus on the mouse chromosome 16 was modified to express the human TMPRSS2 protein. The humanized TMPRSS2 allele was also combined with the humanized FCGRT allele on the C57BL/6J background through breeding.

Guide RNA

The Tmprss2-crRNA_5 guide RNA was developed and tested. The protospacer sequence is 5′-TGTGCTCAGACTGATACCCG-3′ (SEQ ID NO: 17), targeting Cas9 cleavage at codon 19 in exon 3 of the mouse Tmprss2 gene. The synthetic Tmprss2-crRNA_5 crRNA was purchased from IDT and annealed to synthetic tracrRNA (IDT) to produce active guide RNA for genome editing.

Donor Plasmid and Knock-in Allele

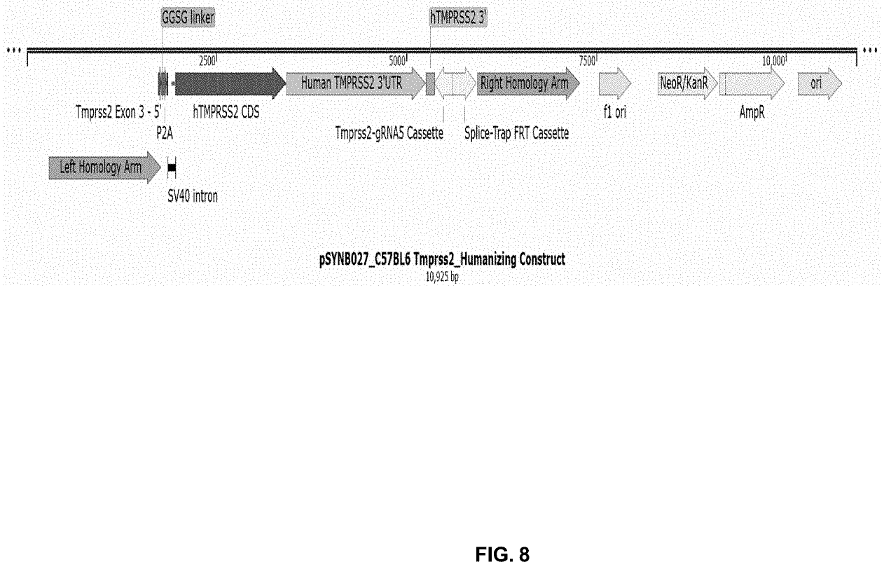

The donor plasmid “pSYNB027_C57BL6_Tmprss2_Humanizing Construct” (SEQ ID NO: 3) was designed and constructed with a total insert size (region between homology arms) of 4161 bp. The plasmid, shown in FIG. 8, contains the following key features:
1. Left Homology Arm: 1481 bp 5′ homology region encompassing sequences immediately 5′ of the Tmprss2-crRNA_5 Cas9 cut site
2. GGSG linker: coding sequence for GGSG linker in-frame with upstream mouse Tmprss2 coding sequence.
3. P2A: porcine teschovirus 2A self-cleaving peptide sequence.
4. hTMPRSS2 CDS: human TMPRSS2 cDNA coding sequence with SV40 intron inserted for enhanced expression
5. hTMPRSS2 3′UTR: 1837 bp human TMPRSS2 3′UTR
6. hTMPRSS2 3′: 112 bp human intergenic sequence immediately 3′ of TMPRSS2 polyadenylation region
7. Tmprss2-gRNA5 Cassette: “Active genetics” cassette comprised of a mouse U6 promoter-driven Tmprss2-gRNA5 guide RNA with 2×PolIII terminator, all in reverse orientation relative to Tmprss2 gene
8. Splice-Trap FRT Cassette: splice acceptor and SV40 polyadenylation sequence followed by a Flp Recombinase Target (FRT) site and 3-frame ATG short open reading frames. This cassette is designed to trap and truncate any mouse Tmprss2 isoforms that may splice past the hTMPRSS2 cassette. The FRT site allows Flp-mediated collapse of targeted tandem integration events if required.
9. Right Homology Arm: 1352 bp 3′ homology arm encompassing sequences immediately 3′ of the Tmprss2-crRNA_5 Cas9 cut site

The donor vector, in glycerol stock, was streaked on an ampicillin plate and a single colony was used to start a maxiprep culture. Maxiprep DNA was analyzed by restriction digestion to confirm the structure and absence of rearrangements prior to microinjection.

### Founder Production Summary

One microinjection session was performed with embryos from C57BL/6J mice as summarized in Table 4. Fourteen pups were born from 173 injected embryos. One female and one male pup had the correct integration of the donor vector and 4 additional animals had integration events that scored positive with vector backbone PCRs, suggesting tandem donor integrations.

TABLE 4
hTMPRSS2 Knock-in, C57BL/6J Strain, Microinjection Summary

| PN Injection (transferred | | |
|---|---|---|
| embryos −> recipients) | Pups | Founder #, Notes |
| 173 1-cell −> 6 | 14 | F#1, M#8 correct knock-ins. |
| | | 4 animals with BB+ knock− |

Founder Genotyping Assays

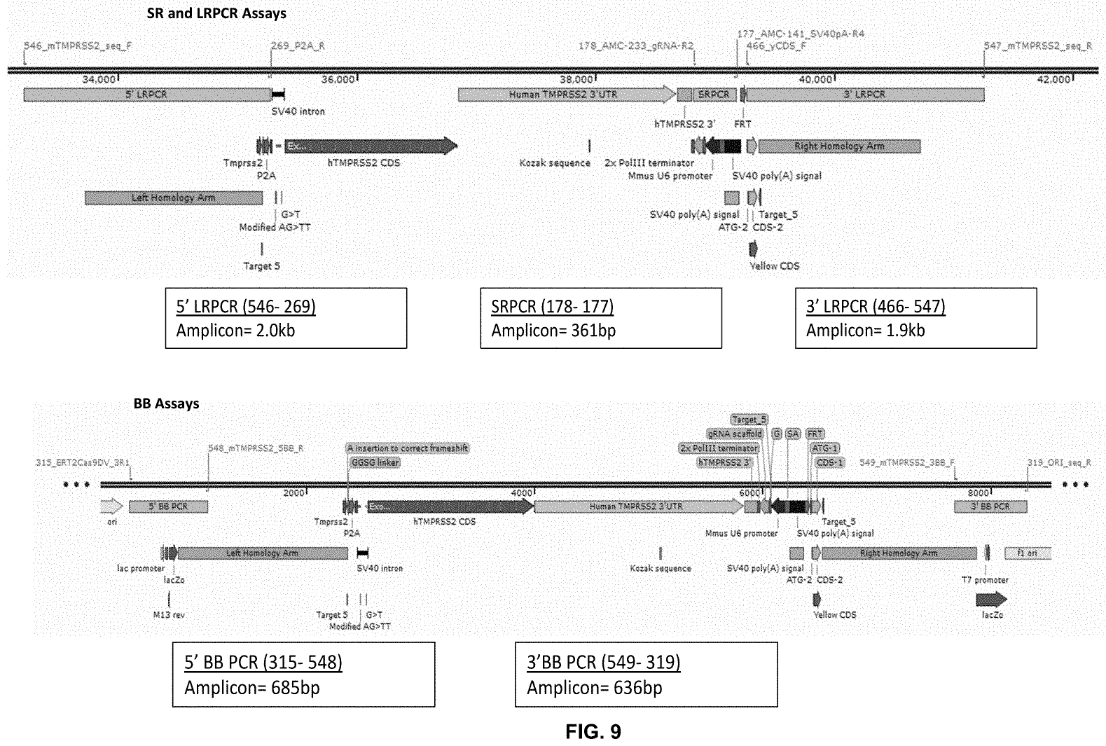

Fourteen potential founder animals were genotyped with the assays depicted in FIG. 9. The primer sequences are shown in Table 5 below. Short range PCR (SRPCR) assays were performed to detect any founders with donor vector integration. 5′ long range PCR (5′ LRPCR) and 3′ long-range PCR (3′ LRPCR) assays were performed with one primer that anneals in native mouse sequence outside the donor vector homology arm and one primer that anneals in sequence unique to the donor vector. Positive amplification by these primers indicates insertion of the transgene at the target locus.

TABLE 5
Primers and Assay Information

| | Fwd | | Rev | | |
|---|---|---|---|---|---|
| | Primer | | Primer | | Amplicon |
| Assay | Name | Fwd Primer Seq | Name | Rev Primer Seq | Size |
| 5′LRPCR | SB546 | GAGGTCCACTCCCTAGTGATTAAG | SB269 | GTCCAGGGTTCTCCTCCACG | 2 kB |
| | | (SEQ ID NO: 18) | | (SEQ ID NO: 19) | |
| 3′LRPCR | SB466 | TGCACCATGGTATCACCATGCAC | SB547 | GGGGGTCATTCTGTACTTCTGG | 1.9 kB |
| | | (SEQ ID NO: 20) | | (SEQ ID NO: 21) | |
| SRPCR | SB177 | AAACCACAACTAGAATGCAGTGA | SB178 | AAAAGCACCGACTCGGTGCCACT | 361 bp |
| | | (SEQ ID NO: 22) | | (SEQ ID NO: 23) | |
| 5′BB | SB315 | CTTTTGCTCACATGTTCTTTCCT | SB548 | GAGCTGTCTTGCTGGCCTAAA | 685 bp |
| | | (SEQ ID NO: 24) | | (SEQ ID NO: 25) | |
| 3′BB | SB319 | CCGTAAAGCACTAAATCGGAACC | SB549 | CCTAACTGATGGCTATTTCTCCAGC | 636 bp |
| | | (SEQ ID NO: 26) | | (SEQ ID NO: 27) | |

5′ and 3′ vector backbone (BB) assays were performed using one primer targeting the homology arm and a second primer targeting the donor vector backbone sequence. BB assays identify random donor integration at off-target sites in the genome as well as tandem integration of multiple donor copies at the target site. Correct targeted homologous integration events exclude the donor vector backbone sequences.

Founder Genotyping Data

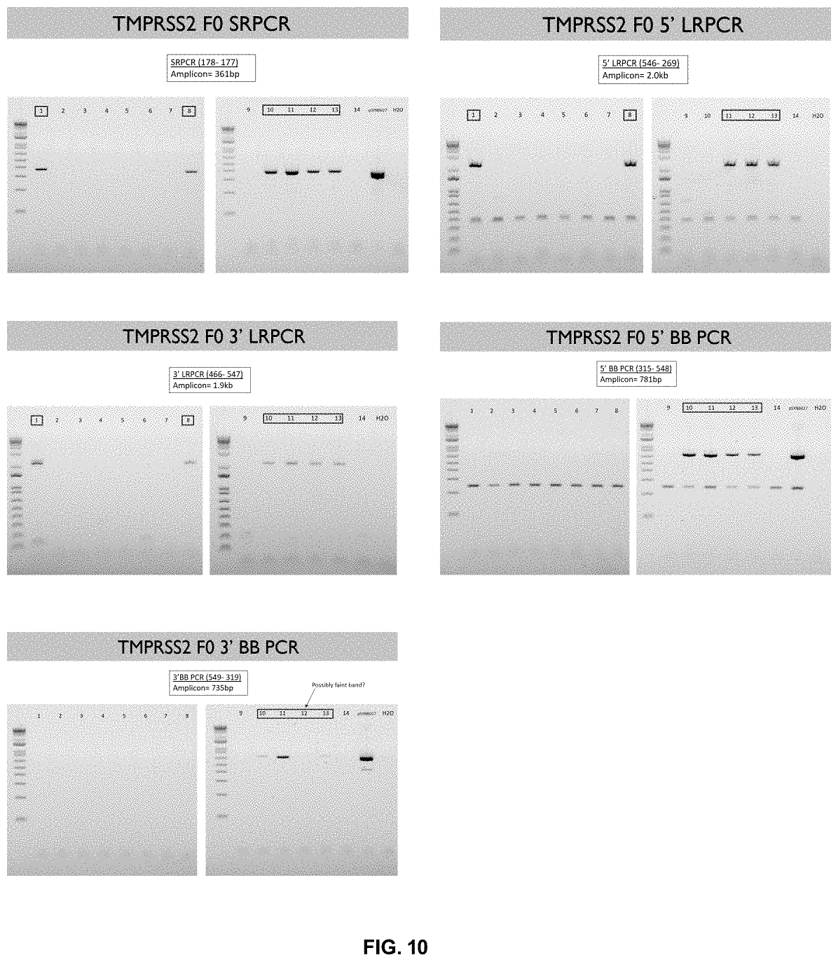

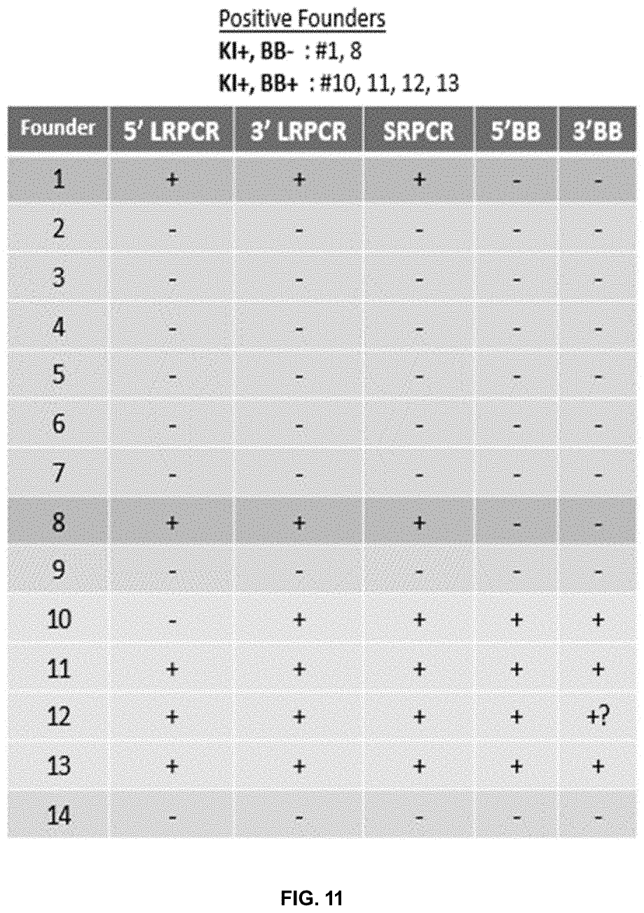

FIG. 10 shows PCR genotyping of potential founder animals 1-14. As shown in FIG. 11, animals 1 and 8 were positive for internal short-range assay and 5′ and 3′ long-range assays, and negative for vector backbone assays. Animals 11, 12, and 13 were positive for all assays, suggesting tandem integration at the target locus. Animal 10 was positive for all except the 5′LRPCR assay, suggesting an aberrant integration event.

Founder Breeding

Vector backbone-negative founders #1 (female) and #8 (male) were mated to animals homozygous for the FCGRT humanized knock-in allele (Fcgrt KI/KI). Both founders transmitted the hTMPRSS2 knock-in event to offspring. However, male #8 unexpectedly gave a high percentage of female offspring (6/6 females), all of which were positive for the hTMPRSS2 knock-in allele. Male #8 was then mated to wild-type C57BL/6J females and produced a litter of 5 females and 2 males, all of which were again positive for the knock-in event. Female #1 displayed a normal offspring sex distribution (9 females, 5 males), with transmission of the hTMPRSS2 knock-in allele to 50% of offspring. Offspring from founder #1 were selected for further breeding and characterization.

Knock-in Allele Validation

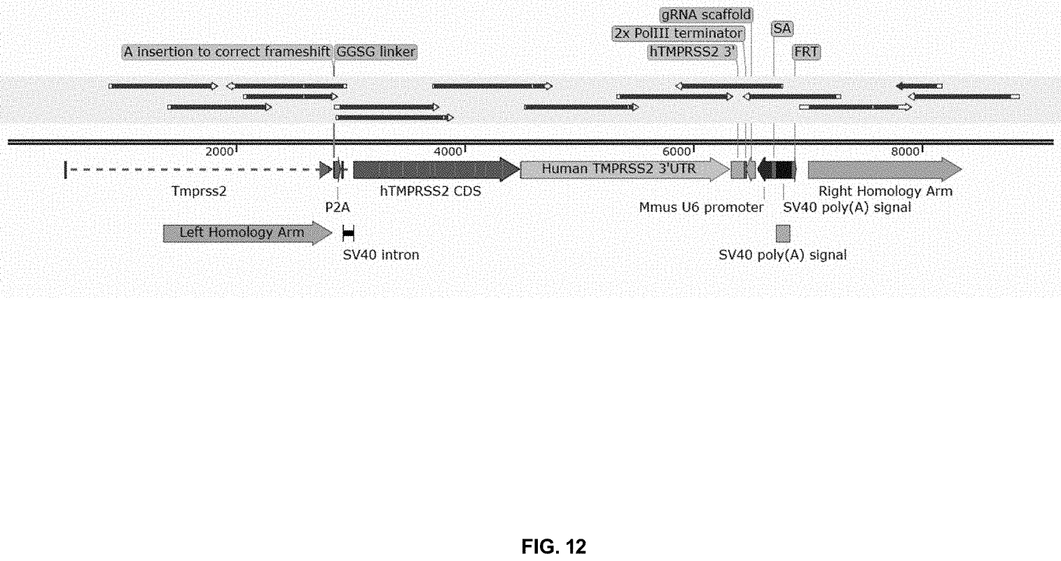

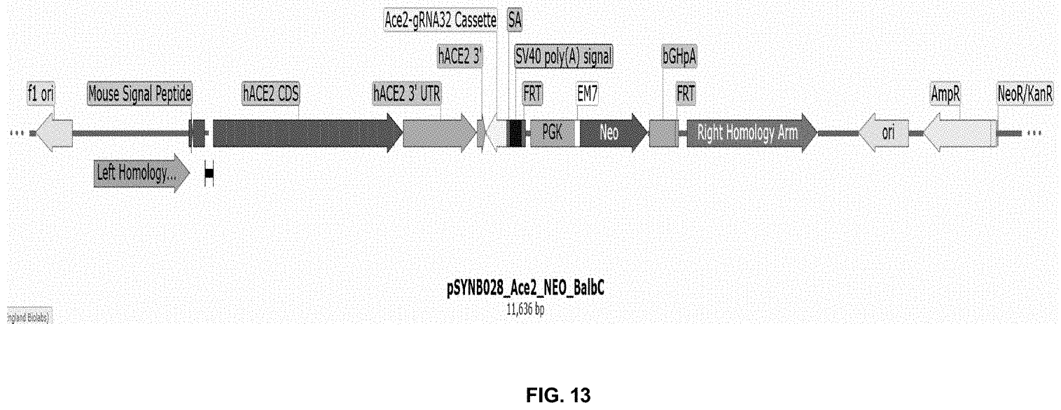

DNA samples from Tmprss2 KI animals were assessed for sequence confirmation of the knock-in allele in founder line 1. Samples from females TMPRSS2-B6 1.7, 1.8 and 1.15 and males TMPRSS2-B6 1.16, 1.60 and 1.61 (genotypes all Tmprss2 KI/+; Fcgrt KI/+) were assessed. The knock-in allele was PCR amplified and amplicons were sequenced. The knock-in allele showed the expected sequence in tested animals, as shown in FIG. 12. A 2 bp deletion was noted in a homopolymeric A repeat in the left homology arm in both the donor vector and the targeted animal when compared to expected c57BL/6J sequence. This change is not expected to have any functional consequences. Bars labeled “PCR1,” “PCR2,” PCR3,” and “PCR4” show PCR amplicons produced and Sanger sequenced from animal TMPRSS2-B6 1.15. The small arrows at the top of the schematic show sequence traces matching expected sequence.

Off-Target Mutation Analysis

The IDT CRISPR-Cas9 Design Checker online tool was used to identify the top five potential off-target cut sites in the mouse genome. Primers were designed to amplify the target regions for sequencing. The off-target sites and primer sequences are shown in FIG. 17B.

DNA samples from animals TMPRSS2-B6 1.60 and 1.61 were used for PCR amplification and sequencing across potential off-target sites. Wild-type control animals were included in the analysis. As shown in Table 6, no mutations were detected in either animal for the 5 sites analyzed.

TABLE 6
Summary of Off-Target Analysis of hTMPRSS2
Animals on C57BL/6 (Black 6) background

TMPRSS2
Black 6

| Off-Target | 1.60 | 1.61 | WT |
|---|---|---|---|
| 1 | NO INDELS | NO INDELS | NO INDELS |
| 2 | NO INDELS | NO INDELS | NO INDELS |
| 3 | NO INDELS | NO INDELS | NO INDELS |
| 4 | NO INDELS | NO INDELS | NO INDELS |
| 5 | NO INDELS | NO INDELS | NO INDELS |

Colony Expansion Breeding

F1 animals harboring the hTMPRSS2 and hFCGRT knock-in alleles were intercrossed to generate hTMPRSS2/hFCGRT double homozygous (KI/KI; KI/KI) animals. Compound heterozygous animals (KI+, KI/+) were later crossed back to C57BL/6J wild-type animals to remove the hFCGRT knock-in allele for a separate colony of animals with only the hTMPRSS2 knock-in allele. Tissue samples were harvested from Tmprss2 KI/KI; Fcgrt KI/KI animals TMPRSS2-B6 1.87 (M, 7 wks old), 1.97 (F, 5 wks old), 1.99 (M, 5 wks old) and 1.103 (F, 3 wks old).

Expression Analysis

Animals are euthanized by CO2 inhalation followed by cervical dislocation. Tissues pieces are placed immediately in 500 μl RNAlater (aqueous solution) at room temperature, then moved to 4° C. Tissues in RNAlater are held at 4° C. at least overnight, and frozen at −80° C. until ready for harvesting.

Tissue Samples for Harvesting
1. Intestine
2. Kidney
3. Prostate
2. Lung divided into 2 pieces ˜100 mg each.
6. Heart collected and divided into 2×˜75 mg pieces.
Transgene expression is assessed.

### Example 3. Development of Humanized BALB/cJ-ACE2 Knock-In Mouse Strain

The Ace2 locus on the mouse X-chromosome was modified to express the human ACE2 protein.

Guide RNA

The Ace2-crRNA_32 guide RNA was developed and tested. The protospacer sequence is 5′-AAGGCTGAGAAGGAGCCAGG-3′ (SEQ ID NO: 28), targeting Cas9 cleavage 13 bp downstream of the mouse Ace2 gene. The synthetic Ace2-crRNA_32 crRNA was purchased from IDT and annealed to synthetic tracrRNA (IDT) to produce active guide RNA for genome editing.

Donor Plasmid and Knock-in Allele

The donor plasmid “pSYNB028_Ace2_NEO_BalbC” (SEQ ID NO: 2) was designed and constructed with a total insert size (region between homology arms) of 5831 bp. The plasmid, shown in FIG. 8, contains the following key features:
1. Left Homology Arm: 1134 bp 5′ homology region encompassing sequences immediately 5′ of the Ace2-crRNA_32 Cas9 cut site
2. Mouse Signal Peptide: signal peptide coding sequence (translation produces amino acid sequence WLLLSLVAVTTA (SEQ ID NO: 6)) in-frame with first 5 amino acids of mouse Ace2
3. hACE2 CDS: human ACE2 cDNA coding sequence with SV40 intron inserted for enhanced expression
4. hACE2 3′UTR: 872 bp human ACE2 3′UTR
5. hACE2 3′: 100 bp human intergenic sequence immediately 3′ of ACE2 polyadenylation region
6. Ace2-gRNA32 Cassette: “Active genetics” cassette comprised of a mouse U6 promoter-driven Ace2-gRNA32 guide RNA with 2×PolIII terminator, all in reverse orientation relative to Ace2
7. SA-SV40 poly(A) signal: splice acceptor and SV40 polyadenylation sequence designed to trap and truncate any mouse Ace2 isoforms that may splice past the hACE2 cassette.
8. FRT-PGK-EM7-Neo-bGHpA-FRT: Neomycin resistance gene driven by the mouse PGK promoter and with a bovine growth hormone polyadenylation sequence. The FRT sites allow Flp-mediated removal of the neomycin cassette from targeted embryonic stem cell clones as well as collapse of targeted tandem integration events if required.
9. Right Homology Arm: 1535 bp 3′ homology arm encompassing sequences immediately 3′ of the Ace2-crRNA_32 Cas9 cut site

The donor vector, in glycerol stock, was streaked on an ampicillin plate and a single colony was used to start a maxiprep culture. Maxiprep DNA was analyzed by restriction digestion to confirm the structure and absence of rearrangements prior to gene targeting in BALB/cJ embryonic stem cells.

### BALB/cJ ES Cell Gene Targeting Summary

BALB/cJ embryonic stem cells (from TransViragen) were nucleofected with 1 μM SpCas9 protein, 1.2 μM Ace2-crRNA_32/tracrRNA and 4 μg supercoiled donor vector. Cells were selected with G418 and individual clones were picked and screened by PCR for integration of the hACE2 knock-in cassette.

ES Cell Screening Assays

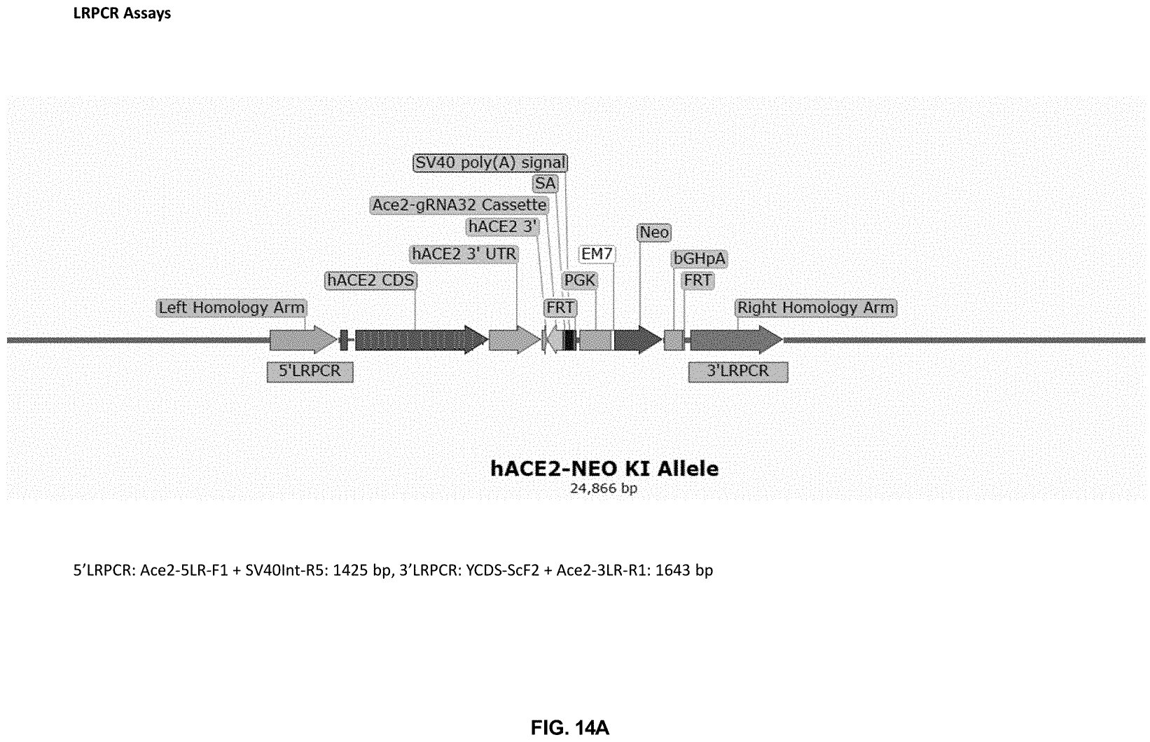

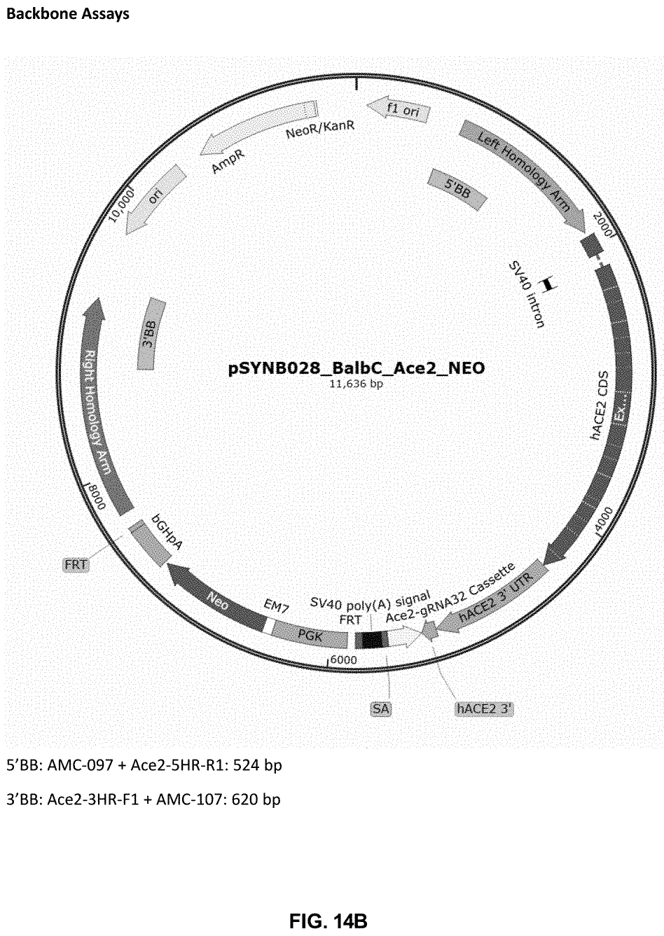

ES cell clones were screened with the PCR assays depicted in FIG. 14 and the primers listed in Table 7.

5′ LRPCR and 3′ LRPCR: Long-range PCR assays with one primer that anneals in native mouse sequence outside the donor vector homology arm and one primer that anneals in sequence unique to the donor vector. Positive amplification by these primers indicates insertion of the transgene at the target locus.

5′ and 3′ vector backbone (BB) assays were performed using one primer targeting the homology arm and a second primer targeting the donor vector backbone sequence. BB assays identify random donor integration at random non-targeted sites in the genome as well as tandem integration of multiple donor copies at the target site. Correct targeted homologous integration events exclude the donor vector backbone sequences. BB assays are performed after a subset of ES cell clones are expanded.

TABLE 7
Primers and Assay Information

| | Fwd | | Rev | | |
|---|---|---|---|---|---|
| | Primer | | Primer | | Amplicon |
| Assay | Name | Fwd Primer Seq | Name | Rev Primer Seq | Size |
| 5′LRPCR | Ace2- | GATGCGCTTTGGATTTCATAATGC | SV40Int- | CACTGAGGAGCAGTTCTTTGATTTGC | 1425 bp |
| | 5LR-F1 | (SEQ ID NO: 29) | R5 | (SEQ ID NO: 30) | |
| 3′LRPCR | YCDS-F2 | AGTTAGGTGTAATCGGCTGAACTACCAG | Ace2- | ACCCTACAAGCATCACACATTCTTCTG | 1643 bp |
| | | (SEQ ID NO: 31) | 3LR-R1 | (SEQ ID NO: 32) | |
| 5′BB | AMC-097 | TAATACGACTCACTATAGGGCGAATTGG | Ace2- | AGGCAAAGGTACTTATTTGTAAAGGAAGC | 524 bp |
| | | (SEQ ID NO: 33) | 5HR-R1 | (SEQ ID NO: 34) | |
| 3′BB | Ace2- | TTGACAATGTAGTTGCCAAATGAGTTG | AMC-107 | TTGTGTGGAATTGTGAGCGGATAAC | 620 bp |
| | 3HR-F1 | (SEQ ID NO: 35) | | (SEQ ID NO: 36) | |
| 5′Probe | Ace2-5PF | TAAAGATGATATGTTCCTTGAGGCCAGT | Ace2-5PR | ACACAATGGGGCACAGCATGTAA | 479 bp |
| | | (SEQ ID NO: 37) | | (SEQ ID NO: 38) | |
| 3′Probe | Ace2-3PF | GTAATTGGATACTGTACCTGGCATTAGTTTG | Ace2-3PR | GCAAACTCACAGTCTAATTCTGCAACTCA | 546 bp |
| | | (SEQ ID NO: 39) | | (SEQ ID NO: 40) | |

ES Cell Southern Blot Data

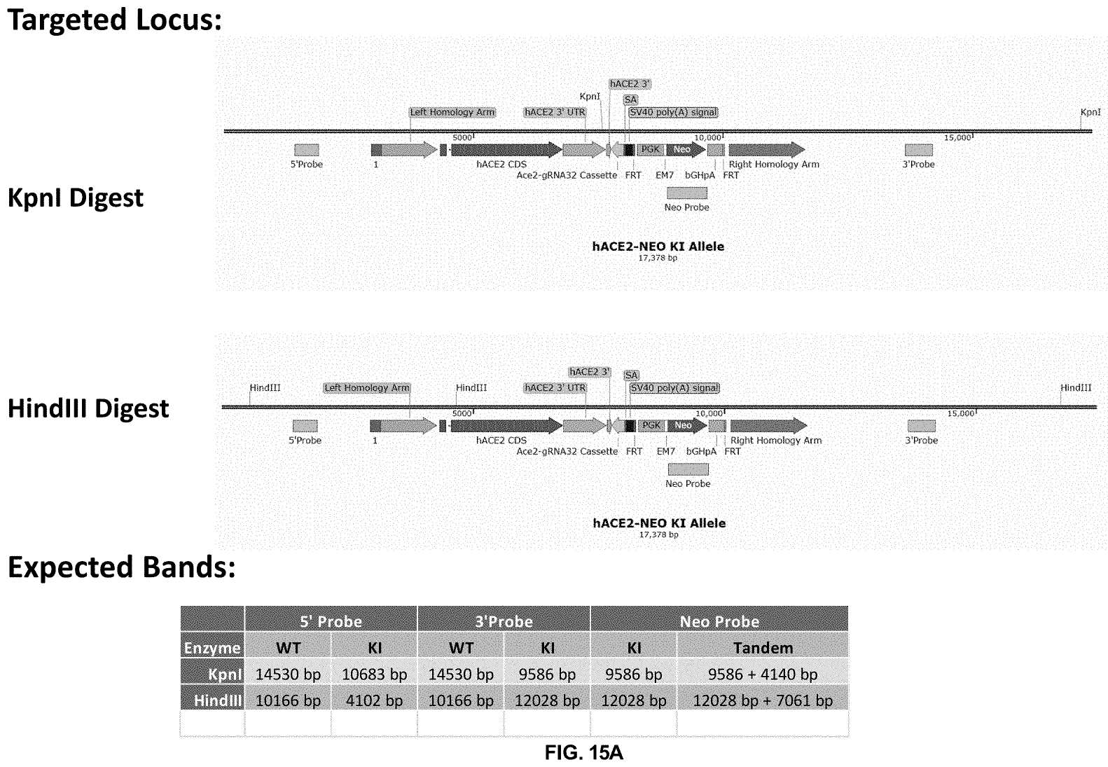

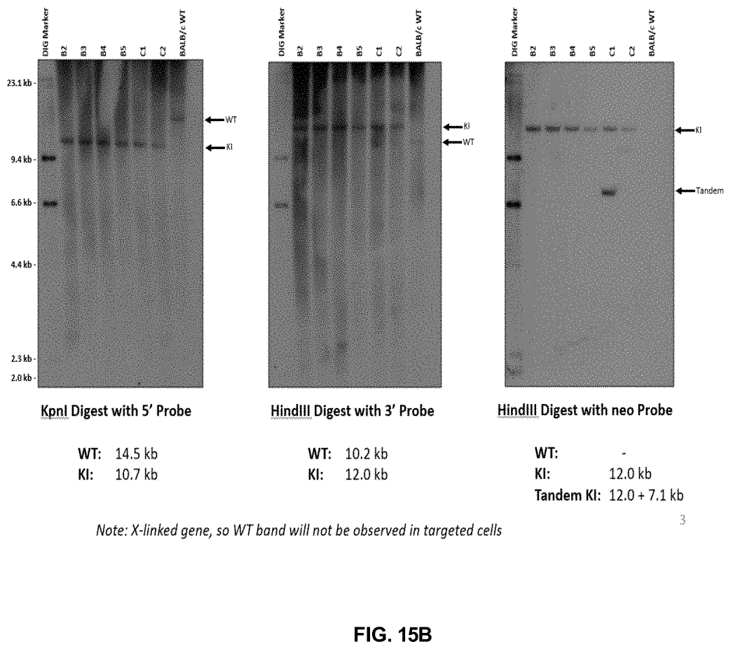

Six PCR-positive ES cell clones were selected for clonal expansion and Southern blot analysis of the hACE2 knock-in allele. FIG. 15 shows the Southern blot strategy and data for the six clones. All six clones showed correct targeting with 5′ and 3′ flanking probes. One clone showed evidence of tandem integration by neo probe. Based on these results, four correct clones were selected for blastocyst injection to produce chimeras for germline transmission of the targeted allele.

Chimera Production

hACE2 targeted ES cell clones B2, B3, B4 and B5 were individually injected into BALB/cJ blastocyst embryos for chimera production. Clones B2 and B3 were each injected into 32 blastocysts, resulting in 15 and 11 pups respectively. Clone B2 only gave 1 low-contribution female chimera and B3 gave 5 low-contribution chimeras (2 females and 3 males). Clones B4 and B5 were injected in 48 and 57 blastocysts, yielding 27 and 7 pups respectively. Clone B4 gave 17 chimeras, including 1 high-contribution, 7 medium-contribution and 8 low-contribution males. Clone B5 gave 7 chimeras including 1 high-contribution and 5 low-contribution males.

Chimera Breeding for Germline Transmission of the hACE Allele

Male chimeras from ES cell clones B3, B4 and B5 were mated to BALB/cJ or transgenic Flpo-BALB/cJ females for germline transmission of the hACE2 allele. Mating to Flpo-BALB/cJ allowed removal of the neomycin cassette concurrent with germline transmission of the targeted allele. One chimera from clone B3 produced a single germline transmission event. Clone B5 produced multiple germline transmission events. Chimeras from clone B4 produced the highest percentage of germline transmission events, and this line was selected for colony expansion and characterization.

Knock-in Allele Validation

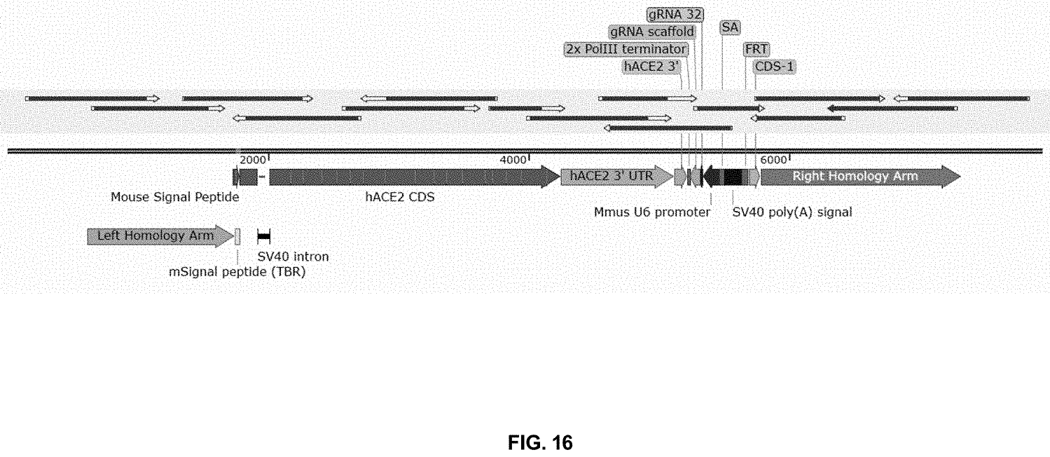

DNA samples from ACE2-C ES cell clone B4 and from two F1 germline transmission animals from clone B4 chimeras were assessed for sequence confirmation of the knock-in allele, as shown in FIG. 16. Animals sampled for this experiment were females ACE2-C B4.13 (genotype Ace2 KI/+; R26+/+) and B4.18 (genotype Ace2 KI/+; R26 Flpo/+). Both females had the hACE2 knock-in allele with neo cassette removed by Flp (maternal effect removal in B4.13), while the ES cell DNA had the neo cassette present. The knock-in allele was PCR amplified and amplicons were sequenced from animal B4.13. The knock-in allele showed the expected sequence. Bars labeled “PCR1,” “PCR2,” PCR3,” and “PCR4” show PCR amplicons produced and Sanger sequenced from animal 2.12. The small arrows at the top of the schematic indicate sequence traces matching expected sequence.

Off-Target Mutation Analysis

The IDT CRISPR-Cas9 Design Checker online tool was used to identify the top five predicted off-target cut sites in the mouse genome. Primers were designed to amplify the target regions for sequencing. The off-target sites and primer sequences are shown in FIG. 17A.

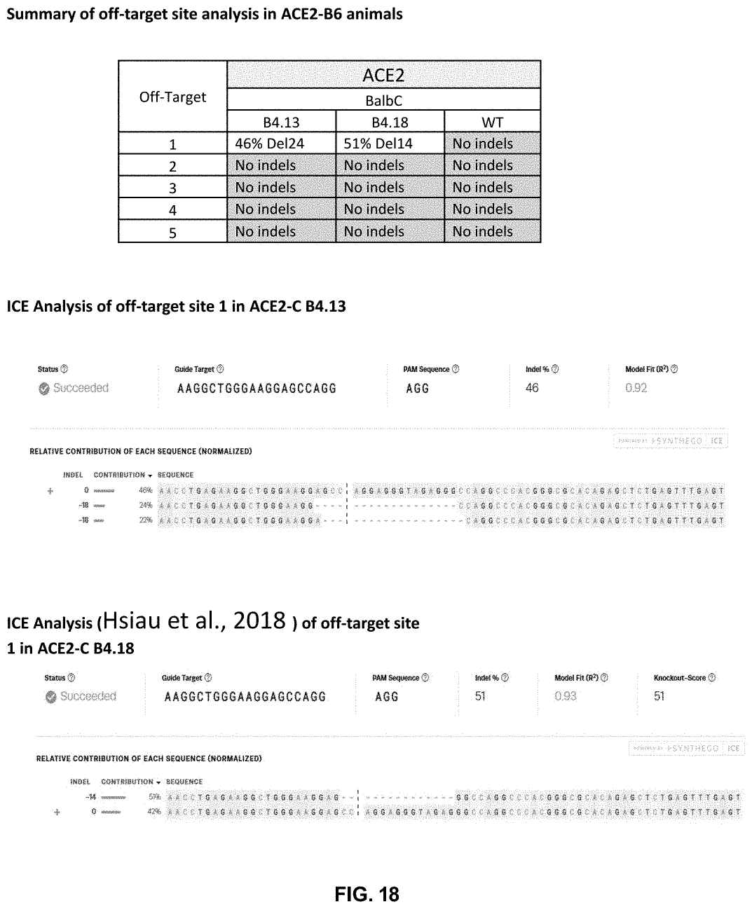

DNA samples from animals ACE2-C B4.13 and B4.18 were used for PCR amplification and sequencing across potential off-target sites. A wild-type control animal was included in the analysis. ACE2-C B4.13 was heterozygous for a 24 bp deletion at off-target site #1, and animal ACE2-C B4.18 was heterozygous for a 14 bp deletion at off-target site 1, as shown in FIG. 18. The presence of two different mutations at off-target site 1 in animals from the same ES cell clone indicates that clone B4 had biallelic mutation of off-target site 1. Off-target site 1 is a non-coding region on chromosome 6, and mutations at the site are not expected to have any phenotypic consequence. Nevertheless, animals in the ACE2-C colony are being actively screened for mutations at the site and breeding strategies were designed to remove the mutant alleles from the colony as a precaution.

Colony Expansion Breeding

Male chimeras only transmitted the X-linked ACE2 knock-in allele to female offspring. F1 females harboring the ACE2 knock-in allele (neo-removed) and Flpo transgene were crossed back to chimeras in some cases to generate homozygous KI/KI females and hemizygous KI/Y males with the hACE2 allele. Females harboring the hACE2-NEO allele (from chimeras crossed to BALB/cJ females) were crossed to Flpo-BALB/c males for production of offspring with the hACE2 allele with neo removed by cotransmission of the Flpo transgene with the hACE2-NEO allele. After homozygous females and hemizygous males were produced, they were intercrossed for further line expansion and, where applicable, to segregate the Flpo transgene away from the hACE2 knock-in allele. Intercross animals are also being screened for mutations at off-target site 1 to identify animals with wild-type sequence for removal of the off-target mutations from the colony. Tissue samples were harvested from ACE2-C B4.127 (Ace2 KIN; R26 Flpo/+).

Expression Analysis

Animals are euthanized by CO2 inhalation followed by cervical dislocation.

Tissues pieces are placed immediately in 500 μl RNAlater (aqueous solution) at room temperature, then moved to 4° C. Tissues in RNAlater are held at 4° C. at least overnight, and frozen at −80° C. until ready for analysis.

Tissue Samples for Harvesting
1. Intestine
2. Kidney
3. Prostate
2. Lung divided into 2 pieces ˜100 mg each.
6. Heart collected and divided into 2×˜75 mg pieces.
Transgene expression are assessed.

### Example 4. Development of Humanized BALB/cJ-TMRSS2 Knock-In Mouse Strain

The Tmprss2 locus on mouse chromosome 16 was modified to express the human TMPRSS2 protein.

Guide RNA

The Tmprss2-crRNA_5 guide RNA was developed and tested. The protospacer sequence is 5′-TGTGCTCAGACTGATACCCG-3′ (SEQ ID NO: 17), targeting Cas9 cleavage at codon 19 in exon 3 of the mouse Tmprss2 gene. The synthetic Tmprss2-crRNA_32 crRNA was purchased from IDT and annealed to synthetic tracrRNA (IDT) to produce active guide RNA for genome editing.

Donor Plasmid and Knock-in Allele

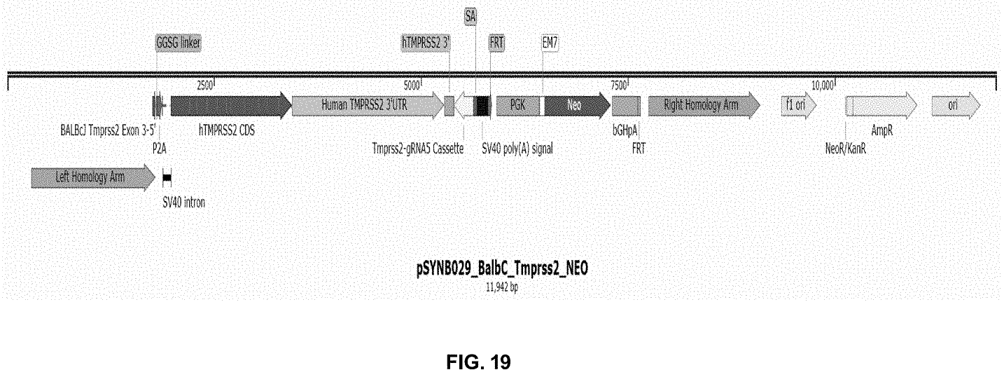

The donor plasmid “pSYNB029_BalbC_Tmprss2_NEO Humanizing Construct” (SEQ ID NO: 4) was designed and constructed with a total insert size (region between homology arms) of 5954 bp. The plasmid, shown in FIG. 19, contains the following key features:
1. Left Homology Arm: 1503 bp 5′ homology region encompassing sequences immediately 5′ of the Tmprss2-crRNA_5 Cas9 cut site
2. GGSG linker: coding sequence for GGSG linker in-frame with upstream mouse Tmprss2 coding sequence.
3. P2A: porcine teschovirus 2A self-cleaving peptide sequence.
4. hTMPRSS2 CDS: human TMPRSS2 cDNA coding sequence with SV40 intron inserted for enhanced expression
5. hTMPRSS2 3′UTR: 1837 bp human TMPRSS2 3′UTR
6. hTMPRSS2 3′: 112 bp human intergenic sequence immediately 3′ of TMPRSS2 polyadenylation region
7. Tmprss2-gRNA5 Cassette: “Active genetics” cassette comprised of a mouse U6 promoter-driven Tmprss2-gRNA5 guide RNA with 2×PolIII terminator, all in reverse orientation relative to Tmprss2 gene
8. SA-SV40 poly(A) signal: splice acceptor and SV40 polyadenylation sequence designed to trap and truncate any mouse Tmprss2 isoforms that may splice past the hTMPRSS2 cassette.
9. FRT-PGK-EM7-Neo-bGHpA-FRT: Neomycin resistance gene driven by the mouse PGK promoter and with a bovine growth hormone polyadenylation sequence. The FRT sites allow Flp-mediated removal of the neomycin cassette from targeted embryonic stem cell clones as well as collapse of targeted tandem integration events if required.
10. Right Homology Arm: 1349 bp 3′ homology arm encompassing sequences immediately 3′ of the Tmprss2-crRNA_5 Cas9 cut site

The donor vector, in glycerol stock, was streaked on an ampicillin plate and a single colony was used to start a maxiprep culture. Maxiprep DNA was analyzed by restriction digestion to confirm the structure and absence of rearrangements prior to gene targeting in BALB/cJ embryonic stem cells.

### BALB/cJ ES Cell Gene Targeting Summary

BALB/cJ embryonic stem cells (TransViragen) were nucleofected with 1 μM SpCas9 protein, 1.2 μM Tmprss2-crRNA_5/tracrRNA and 4 μg supercoiled donor vector. Cells were selected with G418 and individual clones were picked and screened by PCR for integration of the hTMPRSS2 knock-in cassette.

ES Cell Screening Assays

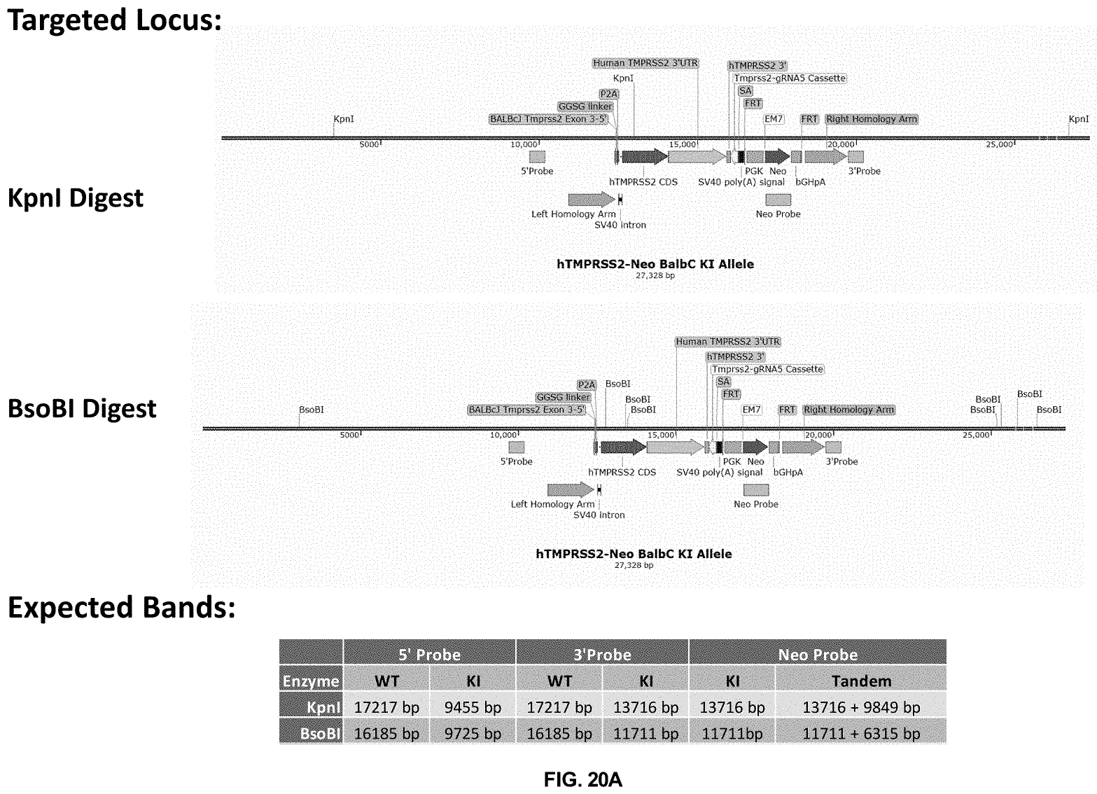

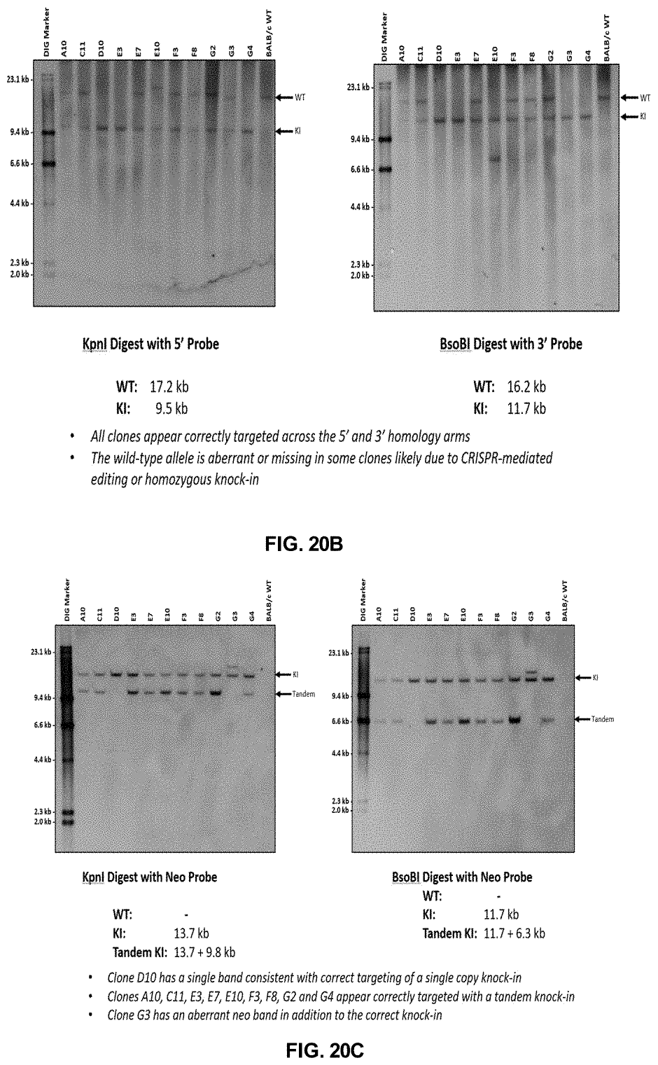

ES cell clones were screened with the PCR assays depicted in FIG. 20 and the primers listed in Table 8.

5′ LRPCR and 3′ LRPCR: Long-range PCR assays with one primer that anneals in native mouse sequence outside the donor vector homology arm and one primer that anneals in sequence unique to the donor vector. Positive amplification by these primers indicates insertion of the transgene at the target locus.

5′ and 3′ vector backbone (BB) assays were performed using one primer targeting the homology arm and a second primer targeting the donor vector backbone sequence. BB assays identify random donor integration at random non-targeted sites in the genome as well as tandem integration of multiple donor copies at the target site. Correct targeted homologous integration events exclude the donor vector backbone sequences. BB assays are performed after a subset of ES cell clones are expanded.

TABLE 8
Primers and Assay Information

| | Fwd | | Rev | | |
|---|---|---|---|---|---|
| | Primer | | Primer | | Amplicon |
| Assay | Name | Fwd Primer Seq | Name | Rev Primer Seq | Size |
| 5′LRPCR | Tmprss2- | GCCAGTGTTCCATTTTATGAGTCTCTG | SV40Int- | CACTGAGGAGCAGTTCTTTGATTTGC | 1733 bp |
| | 5LR-F1 | (SEQ ID NO: 41) | R5 | (SEQ ID NO: 42) | |
| 3′LRPCR | YCDS-F2 | AGTTAGGTGTAATCGGCTGAACTACCAG | Tmprss2- | GGTGGATCAGAGCAAAGAACAGAAGAG | 1451 bp |
| | | (SEQ ID NO: 43) | 3LR-R1 | (SEQ ID NO: 44) | |
| 5′BB | AMC-107 | TTGTGTGGAATTGTGAGCGGATAAC | Tmprss2- | TGTCTTGCTGGCCTAAAGGTTGAGA | 400 bp |
| | | (SEQ ID NO: 45) | 5HR-R1 | (SEQ ID NO: 46) | |
| 3′BB | Tmprss2- | AGGACACCAGGGACTCTATTGATAAGC | AMC-097 | TAATACGACTCACTATAGGGCGAATTGG | 490 bp |
| | 3HR-F1 | (SEQ ID NO: 47) | | (SEQ ID NO: 48) | |
| 5′Probe | Tmprss2- | CTGCCTGTCCCCATTTCTTCAAG | Tmprss2- | GCACCTGCTCCTACACACAGTTCTTAAA | 475 bp |
| | 5PF | (SEQ ID NO: 49) | 5PR | (SEQ ID NO: 50) | |
| 3′Probe | Tmprss2- | TGCTGTCCCTCTCTACTTCTGAGTGG | Tmprss2- | GAAGCCCAAGACTGTAGTCTGAATGG | 484 bp |
| | 3PF | (SEQ ID NO: 51) | 3PR | (SEQ ID NO: 52) | |

ES Cell Southern Blot Data

Eleven PCR-positive ES cell clones were selected for clonal expansion and Southern blot analysis of the hTMPRSS2 knock-in allele. FIG. 20A shows the Southern blot strategy and data for the eleven clones. The 5′ and 3′ probes flanking the insertion region showed a band of the expected size for all eleven clones, as shown in FIG. 20B. The “wild-type” (non-knock-in) band was absent in some clones, suggesting bi-allelic knock-in of the hTMPRSS2 construct. In other clones the “wild-type” band was an unexpected size, suggesting the presence of CRISPR-mediated insertion or deletion (indel) events in the non-knock-in allele, as shown in FIG. 20C. The Neo probe also showed a band of the expected size for all eleven clones (FIG. 20C). Nine of the eleven clones also showed a second band of the size expected for tandem multimeric insertion events at the target locus. One clone showed a band size suggesting an aberrant integration event.

Based on the Southern blot results, clones D10 (biallelic targeted, no tandem integration) and G4 (biallelic targeted, tandem integration) were selected for blastocyst injection to produce chimeras for germline transmission of the targeted allele. In the case of tandem integration, the crosses to Flp recombinase animals required for neomycin cassette removal also collapse tandem integration events to single-copy.

Chimera Production

hTMPRSS2 targeted ES cell clones D10 and G4 were individually injected into C57BL/6J blastocyst embryos for chimera production. Clone D10 was injected into 20 blastocysts, resulting in 12 pups, with 5 chimeras (1 medium-contribution male, 2 each low-contribution males and females). Clone G4 was injected in 16 blastocysts and yielded 9 pups, with 7 chimeras (1 high-contribution male, 1 each medium contribution male and female, 3 low-contribution males and 1 low-contribution female).

Chimera Breeding for Germline Transmission of the hTMPRSS2 Allele

Chimeras from both D10 and G4 ES cell clones were mated to BALB/cJ or transgenic Flpo-BALB/cJ animals for germline transmission of the hTMPRSS2 allele. Mating to Flpo-BALB/cJ allowed removal of the neomycin cassette concurrent with germline transmission of the targeted allele and removal of tandem multimers. Chimeras from clone D10 failed to transmit the hTMPRSS2 allele through the germline. Three chimeras from clone G4 each produced multiple germline transmission events. The G4 line was selected for colony expansion and characterization.

Knock-in Allele Validation

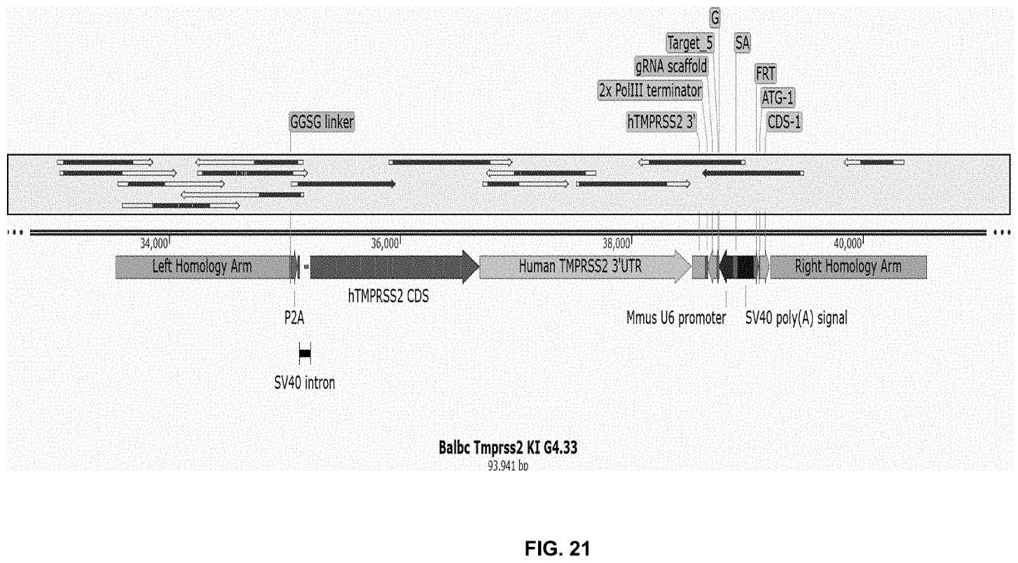

DNA samples from TMPRSS2-C ES cell clone G4 and animals TMPRSS2-C animals G4.32 (female, Tmprss2 KI/+) and G4.33 (male, Tmprss2 KI/+; R26 Flpo/+) were assessed for sequence confirmation of the knock-in allele. The knock-in allele was PCR amplified and amplicons were sequenced (FIG. 21). Additional sequencing confirms the full insert region. The small arrows at the top of the schematic indicate sequence traces matching expected sequence.

Off-Target Mutation Analysis

The IDT CRISPR-Cas9 Design Checker online tool was used to identify the top five predicted off-target cut sites in the mouse genome. Primers were designed to amplify the target regions for sequencing. The off-target sites and primer sequences are shown in FIG. 17B.

DNA samples from animal TMPRSS2-C G4.33 and from ES cell clone G4 were used for PCR amplification and sequencing across potential off-target sites. Wild-type control DNA was included in the analysis. No mutations were detected in the animal or ES cell clone for the 5 sites analyzed as shown in Table 9.

TABLE 9
Summary of Off-Target Analysis of hTMPRSS2 Animals
and ES cell clone on BALB/cJ background

TMPRSS2
BalbC

| Off-Target | G4.33 | G4 ES cell | WT |
|---|---|---|---|
| 1 | NO INDELS | NO INDELS | NO INDELS |
| 2 | NO INDELS | NO INDELS | NO INDELS |
| 3 | NO INDELS | NO INDELS | NO INDELS |
| 4 | NO INDELS | NO INDELS | NO INDELS |
| 5 | NO INDELS | NO INDELS | NO INDELS |

Colony Expansion Breeding

Animals harboring the hTMPRSS2 knock-in allele with neo cassette removed (KI/+) with or without the Flpo transgene are intercrossed to produce hTMPRSS2 homozygous (KI/KI) animals for rapid colony expansion and characterization. As the colony expands, breeding strategies are adjusted to remove the Flpo transgene and produce animals with only the knock-in allele. Tmprss2 KI/KI; R26 Flpo/+animals TMPRSS2-C G4.164 (female, 9 wks old) and G4. 192 (male, 5 wks old) are analyzed for gene expression.

Sequences

Sequences for the constructs described herein are provided below. The sequences are all given in 5′—>3′.

SYNB026_C57BL6_Ace2_Humanizing Construct (SEQ ID NO: 1)
1. Left Homology Arm: 1134 bp 5′ homology region encompassing sequences immediately
5′ of the Ace2-crRNA_32 Cas9 cut site
GAGTTCTCAGCTGAGTTGTAAGCAGGTAAGTGAAAGGGAAAGAGGCACCTGATAAA
GTCAGCTGTAGGACTAGAGTTTAGCATGTCTCCTGAGAAATAGAAAATGACTGCTTG
AAACTTTACCAAAGCCACACAGGAAGCATCAAACTCCCTATGGAGTGGAGAAGAGT
CTTATAATTTTTTAAATGGGCAGAGAAATGAATTTATTTTTAATTTTTAGAGACAGG
GTTTCTTTGTATAGCTCTAGCTGTCTTTGATTGGTAGACAAAGCTGTCCTCAAACTCA
GAGATCTTCCTTCCTTTGTCTCCTGAGTGCTGGGATTAAAGGCATGGACCACCACTG
CCCTGCCCCATTCTCTCCATTAATTTTAAGTGAATGCTTGCAAAAGCTCACTTCTTTG
GTGAACAGCTTCCTTTACAAATAAGTACCTTTGCCTTCGTTTTTATAGGATTCTTAAA
AAGAAAAAAAAGATTCAGCCAGGTGGTTGTGGTGCACACCTTTAATCCCAGCAGTC
AGGAGGCAGAGGAAAGCAGATCTCTTGAGTTTGAGGCTAGCCTAGTCTACAGAGGG
AGTTCCAGGACAGCCAAGGCTACAGAGAGGAACTGTCTAAAAACACCAAGAAAGA
GAGAAAGGAGAGAGGGAGAGGATGGATAGCTTATTGATAGAATTGTCAGAAAAGG
CTATAAGTTCCAATATGTGTCCCATGATTTCTAAGTCTAGCCCTTTCTGTTATAGTAA
AATCATAGTACACCCTCCTCCTCCAGTGTATCTTTAACAGCTTTTAAGGAACATATTA
ACTAAATGTCCAGGTTTTGATTTGGCCATAAAATGTTAGCAAAGCTAAGGTTTTCTA
GGATTAATGAATAACATGTCTTTATTTAGTTTACTTAAAAAAATCATTCTAAAATATC
TGTTTACATATCTGTCCTCTCCAGGATTAACTTCATATTGGTCCAGCAGCTTGTTTAC
TGTTCTCTTCTGTTTCTTCTTCTGCTTTTTTTTTCTTCTCTTCTCAGTGCCCAACCCAAG
TTCAAAGGCTGATGAGAGAGAAAAACTCATGAAGAGATTTTACTCTAGGGAAAGTT
GCTCAGTGGATGGGATCTTGGCGCACGGGGAAAGATGTCCAGCTCCTCCT (SEQ ID
NO: 53)
2. Mouse Signal Peptide: signal peptide coding sequence (translation produces amino acid
sequence WLLLSLVAVTTA (SEQ ID NO: 54)) in-frame with first 5 amino acids of mouse Ace2
Sequence: ATGTCCAGCTCCTCCTGGCTTCTGCTTTCTCTGGTGGCAGTGACAACAGCA
(SEQ ID NO: 55)
3. hACE2 CDS: human ACE2 cDNA coding sequence with SV40 intron inserted for
enhanced expression
Sequence:
CAGTCCACCATTGAGGAACAGGCCAAGACATTTTTGGACAAGTTTAACCACGAAGC
CGAAGACCTGTTCTATCAAAGTTCACTTGCTTCTTGGAATTATAACACCAATATTACT
GAAGAGAATGTCCAAAACATGGTAAGTTTAGTCTTTTTGTCTTTTATTTCttGTCCCGG
ATCCGGTGGTGGTGCAAATCAAAGAACTGCTCCTCAGTGGATtTTGCCTTTACTTCTA
GAATAATGCTGGGGACAAATGGTCTGCCTTTTTAAAGGAACAGTCCACACTTGCCCA
AATGTATCCACTACAAGAAATTCAGAATCTCACAGTCAAGCTTCAGCTGCAGGCTCT
TCAGCAAAATGGGTCTTCAGTGCTCTCAGAAGACAAGAGCAAACGGTTGAACACAA
TTCTAAATACAATGAGCACCATCTACAGTACTGGAAAAGTTTGTAACCCAGATAATC
CACAAGAATGCTTATTACTTGAACCAGGTTTGAATGAAATAATGGCAAACAGTTTAG
ACTACAATGAGAGGCTCTGGGCTTGGGAAAGCTGGAGATCTGAGGTCGGCAAGCAG
CTGAGGCCATTATATGAAGAGTATGTGGTCTTGAAAAATGAGATGGCAAGAGCAAA
TCATTATGAGGACTATGGGGATTATTGGAGAGGAGACTATGAAGTAAATGGGGTAG
ATGGCTATGACTACAGCCGCGGCCAGTTGATTGAAGATGTGGAACATACCTTTGAAG
AGATTAAACCATTATATGAACATCTTCATGCCTATGTGAGGGCAAAGTTGATGAATG
CCTATCCTTCCTATATCAGTCCAATTGGATGCCTCCCTGCTCATTTGCTTGGTGATAT
GTGGGGTAGATTTTGGACAAATCTGTACTCTTTGACAGTTCCCTTTGGACAGAAACC
AAACATAGATGTTACTGATGCAATGGTGGACCAGGCCTGGGATGCACAGAGAATAT
TCAAGGAGGCCGAGAAGTTCTTTGTATCTGTTGGTCTTCCTAATATGACTCAAGGAT
TCTGGGAAAATTCCATGCTAACGGACCCAGGAAATGTTCAGAAAGCAGTCTGCCAT
CCCACAGCTTGGGACCTGGGGAAGGGCGACTTCAGGATCCTTATGTGCACAAAGGT
GACAATGGACGACTTCCTGACAGCTCATCATGAGATGGGGCATATCCAGTATGATAT
GGCATATGCTGCACAACCTTTTCTGCTAAGAAATGGAGCTAATGAAGGATTCCATGA
AGCTGTTGGGGAAATCATGTCACTTTCTGCAGCCACACCTAAGCATTTAAAATCCAT
TGGTCTTCTGTCACCCGATTTTCAAGAAGACAATGAAACAGAAATAAACTTCCTGCT
CAAACAAGCACTCACGATTGTTGGGACTCTGCCATTTACTTACATGTTAGAGAAGTG
GAGGTGGATGGTCTTTAAAGGGGAAATTCCCAAAGACCAGTGGATGAAAAAGTGGT
GGGAGATGAAGCGAGAGATAGTTGGGGTGGTGGAACCTGTGCCCCATGATGAAACA
TACTGTGACCCCGCATCTCTGTTCCATGTTTCTAATGATTACTCATTCATTCGATATT
ACACAAGGACCCTTTACCAATTCCAGTTTCAAGAAGCACTTTGTCAAGCAGCTAAAC
ATGAAGGCCCTCTGCACAAATGTGACATCTCAAACTCTACAGAAGCTGGACAGAAA
CTGTTCAATATGCTGAGGCTTGGAAAATCAGAACCCTGGACCCTAGCATTGGAAAAT
GTTGTAGGAGCAAAGAACATGAATGTAAGGCCACTGCTCAACTACTTTGAGCCCTTA
TTTACCTGGCTGAAAGACCAGAACAAGAATTCTTTTGTGGGATGGAGTACCGACTGG
AGTCCATATGCAGACCAAAGCATCAAAGTGAGGATAAGCCTAAAATCAGCTCTTGG
AGATAAAGCATATGAATGGAACGACAATGAAATGTACCTGTTCCGATCATCTGTTGC
ATATGCTATGAGGCAGTACTTTTTAAAAGTAAAAAATCAGATGATTCTTTTTGGGGA
GGAGGATGTGCGAGTGGCTAATTTGAAACCAAGAATCTCCTTTAATTTCTTTGTCAC
TGCACCTAAAAATGTGTCTGATATCATTCCTAGAACTGAAGTTGAAAAGGCCATCAG
GATGTCCCGGAGCCGTATCAATGATGCTTTCCGTCTGAATGACAACAGCCTAGAGTT
TCTGGGGATACAGCCAACACTTGGACCTCCTAACCAGCCCCCTGTTTCCATATGGCT
GATTGTTTTTGGAGTTGTGATGGGAGTGATAGTGGTTGGCATTGTCATCCTGATCTTC
ACTGGGATCAGAGATCGGAAGAAGAAAAATAAAGCAAGAAGTGGAGAAAATCCTT
ATGCCTCCATCGATATTAGCAAAGGAGAAAATAATCCAGGATTCCAAAACACTGAT
GATGTTCAGACCTCCTTTTAG (SEQ ID NO: 56)
4. hACE2 3′UTR: 872 bp human ACE2 3′UTR
AAAAATCTATGTTTTTCCTCTTGAGGTGATTTTGTTGTATGTAAATGTTAATTTCATG
GTATAGAAAATATAAGATGATAAAGATATCATTAAATGTCAAAACTATGACTCTGTT
CAGAAAAAAAATTGTCCAAAGACAACATGGCCAAGGAGAGAGCATCTTCATTGACA
TTGCTTTCAGTATTTATTTCTGTCTCTGGATTTGACTTCTGTTCTGTTTCTTAATAAGG
ATTTTGTATTAGAGTATATTAGGGAAAGTGTGTATTTGGTCTCACAGGCTGTTCAGG
GATAATCTAAATGTAAATGTCTGTTGAATTTCTGAAGTTGAAAACAAGGATATATCA
TTGGAGCAAGTGTTGGATCTTGTATGGAATATGGATGGATCACTTGTAAGGACAGTG
CCTGGGAACTGGTGTAGCTGCAAGGATTGAGAATGGCATGCATTAGCTCACTTTCAT
TTAATCCATTGTCAAGGATGACATGCTTTCTTCACAGTAACTCAGTTCAAGTACTATG
GTGATTTGCCTACAGTGATGTTTGGAATCGATCATGCTTTCTTCAAGGTGACAGGTCT
AAAGAGAGAAGAATCCAGGGAACAGGTAGAGGACATTGCTTTTTCACTTCCAAGGT
GCTTGATCAACATCTCCCTGACAACACAAAACTAGAGCCAGGGGCCTCCGTGAACT
CCCAGAGCATGCCTGATAGAAACTCATTTCTACTGTTCTCTAACTGTGGAGTGAATG
GAAATTCCAACTGTATGTTCACCCTCTGAAGTGGGTACCCAGTCTCTTAAATCTTTTG
TATTTGCTCACAGTGTTTGAGCAGTGCTGAGCACAAAGCAGACACTCAATAAATGCT
AGATTTACACACTC (SEQ ID NO: 57)
5. hACE2 3′: 100 bp human intergenic sequence immediately 3′ of ACE2 polyadenylation
region
CTTGTGCTTACTTATGTGCTGGGGCTTCTTTACGTTTTGTCTGCTTTTCAGTTCTATGA
ACAAAGTTATCATCTGAGTGTCTGGGGCTCCTGGCCTTCCA (SEQ ID NO: 58)
6. Ace2-gRNA32 Cassette: “Active genetics” cassette comprised of a mouse U6 promoter-
driven Ace2-gRNA32 guide RNA with 2xPolIII terminator, all in reverse orientation relative
to Ace2
AAAAAAAGTGCCTAGCAAAAAAGCACCGACTCGGTGCCACTTTTTCAAGTTGATAA
CGGACTAGCCTTATTTTAACTTGCTATTTCTAGCTCTAAAACCCTGGCTCCTTCTCAG
CCTTCAAACAAGGCTTTTCTCCAAGGGATATTTATAGTCTCAAAACACACAATTACT
TTACAGTTAGGGTGAGTTTCCTTTTGTGCTGTTTTTTAAAATAATAATTTAGTATTTG
TATCTCTTATA (SEQ ID NO: 59)
7. Splice-Trap FRT Cassette: splice acceptor and SV40 polyadenylation sequence followed
by a Flp Recombinase Target (FRT) site and 3-frame ATG short open reading frames. This
cassette is designed to trap and truncate any mouse Ace2 isoforms that may splice past the
hACE2 cassette. The FRT site allows Flp-mediated collapse of targeted tandem integration
events if required.
AATATTTTTTTCCCTCTCTAGCTTTACAATAATTTTCCCAGAACTTGTTTATTGCAGCT
TATAATGGTTACAAATAAAGCAATAGCATCACAAATTTCACAAATAAAGCATTTTTT
TCACTGCATTCTAGTTGTGGTTTGTCCAAACTCATCAATGTATCTTATCATGTCTGGA
TGAAGTTCCTATTCCGAAGTTCCTATTCTCTAGAAAGTATAGGAACTTCTGCACCAT
GGTATCACCATGCACTCACCATGGGAGATTGCGCCAACAGTATTACCACTGCCTAGC
GCGTAGTTAGGTGTAATCGGCTGAACTACCAGCGGCCG (SEQ ID NO: 60)
8. Right Homology Arm: 1535 bp 3′ homology arm encompassing sequences immediately 3′
of the Ace2-crRNA_32 Cas9 cut site
CCTTCTCAGCCTTGTTGCTGTTACTACTGCTCAGTCCCTCACCGAGGAAAATGCCAA
GACATTTTTAAACAACTTTAATCAGGAAGCTGAAGACCTGTCTTATCAAAGTTCACT
TGCTTCTTGGAATTATAATACTAACATTACTGAAGAAAATGCCCAAAAGATGGTAAG
TTCTTGAGGCTACCCAGGGGGTTATTGATTGCTTCTTAAAGATCAGAATTACTGCCT
ATAAAACTGGATAAGGAAATCATAGAGATCTCTCAAGTGTGAGGATGAGTGACTGC
CTCTGTAGCTCTGATCCTAGTCTCCCAGATGGCTAAATTCAATTGACCTTAGAGTTCA
TCTGGAAAATTGTTATGAATGAATTATTTGCCCAGATTCCAAAGATGAGTGAAAATG
TTTAATAAAGTTGCCATCACTATTCTCATTATATTTGGTATGTAAAGCATTCATGGAA
ATGTTCTAAGTCGTTATTGAGCCAATAATTTTCTTTAGCTTATAATGCCAACAGGTCT
ATCCGAGAACTACAAATGACATATTAACTGAAAAATGCAACTGGGGTTTACTGAAG
GCAGCAGCTTAGTAATTAAGGTAACCATGGCTTAGGTGAAACTGGACCTGGGAATT
CCTTCTTTCATTGACACAGAGCTCTGAGGAATTTCCAAAGGTCACAGAAGAAAAGCT
ATAATTAAACTAGTCCCAAAAAATCTCAGCCTACTCTGGGAAAGCAGCATATTTTGT
TTGACAAGTGCAAGGACTTAGAACTTTTTTTTTTCTCACTGATCCTGAAGTGCCTTTT
AAGTATAGTTAAGTGGTGGAAAATTGAGCAACTATTTAAGAAAAGACTCTTTTTTTT
cttcttccagcaatgctttccttcaaaacggtagcttcaaaacttcctgtcttttaaa
TGATCAGGGGGCTGTGTGTTTAAATTATTGCCATTCATAGAACAGAGTGGGTCTGAG
GATGCCTGTTTCCTTTGAAATTCTATGCCCCCTCCCAGTTTTCTAAAATTTAAGAAAC
CACAGAGACTTTGACAATGTAGTTGCCAAATGAGTTGCTTTTAACTGCTCTAATAGT
TTGGTCTTACCGGTGTTGTTTTTAGTGTAACTTTTTTTCCCCTCAGTTTTTATTTTATTT
CTGTTGGAGATCACCAACGTGTACTGTAGATTTTCTGAGCTGAATGGTTCAAGGTGA
GACTAATATTATACTTGAAAGCCTAGCCTGGAGCTGACATCCAGATCCTGGCTGTAC
CACTTTTTAGCTGGGTAAACTTGAATTCATTAAATGGACAGTCTGGGTCTTAGTTACT
TTTTGTTTTAAGATGTATTTTAATTTTAAAACTATGTGTATGTGTGTGTGCTCCCGAA
TATAAGTATAGATGCCCATGAAGGTCAGAGATATTGGAACCCCCAGGAGTGGTGCA
GCAGGCTATTGTGTGCCACCAGACCTAGATGCTGGCAACTTGCAACCTCTGCAAGGG
AAGTACATATCCTTAACCATCGAGCCACCTTTCTACCCCAGTCTTA (SEQ ID NO: 61)
pSYNB027_C57BL6_Tmprss2_Humanizing Construct (SEQ ID NO: 3)
1. Left Homology Arm: 1481 bp 5′ homology region encompassing sequences immediately
5′ of the Tmprss2-crRNA_5 Cas9 cut site
GTCATCCAGGACAAAGTGCTAGGTGTATCTTGTTCTTTCCTGCTGGAGAGGGGAACA
CATTCTTAGACACCTGGTTTCTCAATCTGGAATGCTCTGTTAATATGGGAATAGCTTT
CGCTGTCCCACCCTGAGGTTTCAAGTTATAATCTTTGGACCTTTGAAGGAGAGTGCT
AGTGAGTTATTAAGCACAGATGATTAACCTTGGAGTGTTGGTATTGCCTTAAAATTC
TCAACCTTTAGGCCAGCAAGACAGCTCCATAAGGTAAGAGATGACCTGCATTCAATC
TTTGTGGCCACAGGGTGGAAGGAGACAACTGGCTCTCACAGGTTGTCCTATGATCTC
TGGTTACACACACGTGCAACACACACAATAAATACATGAGCTTTTAAAAGTCTGAGC
ATCCAGTGGTCCCTGCTGAGGATATCTGCTTCCAGAAGATGATTTTTCTGTCATTCCT
GAGGCCCATGAGAGAACCTGAGCAGAAAGAGAGCTGGAGAGGTTTGGGGTAAGAA
CCATTAGACATGCATAGGGAGACCTCTCTGGGAGAGGTCCAGCTACTGAGAGTAGG
GAGAGTGGAAATAACAATGCCAACCTGTGAACTTGCTACTGAACCTGACAACTGCT
GGGCCATACTAGGAGTACAGTCTGGGGAAGGTCACGTGAATGGACTGAGTGAAAGG
ATTGATACCCAAGAAGTGGATCCTGCCCTTTTTACATTCACACAGCACTTACTTTGAC
CTCCAAGTTCCCCTGAAAGGGCACCTACACCCTCAGCACCAAAATGCATACACTCTA
TAGGGATGTTCACACTGAGCTAACACCATCCTGGTGAGAACAGATGGGTAAAGGAC
TACCAACTACAGCTGCATGAGAGAAGGACCCTGGAAGAGAATGGCCACAGAGTCTG
CATTGTGGGTCCCTTCTAGCAGTTTCTCAGCTCAGCTGAAAAGTGTATTTACTACACC
CAAGGTTGCACACATTATCTGAATATGAGGTCCCTTTAGCAGGTGAGTTCTCAGATA
TGAGCTATATCCTGAACCTTGACATTAACCTGGCTCAAAATTTCAGACCTCAAGGCT
CAGGGGGTGATTTAGAGGAGGAACCAGAGAATGCGACTCTAACTGTGTGACTTTTG
ACCCCAAACTTGTATGTATGTTTTCTACAGAATCTTAGTTATCGGTCACCTTTTCTTT
ATTTCAAAGAACAAATAATGAAAAAAAAAAAATGGTCCAGGAAACCAAAGCTAAT
GAAACACAGATAGTTGAGCGTTCTAGTTAGTGCAGAAACAAGAGTCTAGTCATGTT
AAGCAAGTTGCTGTGATGACAGAGTGATTGGCAATCTGCTGGCTCTCTCCCTGTTTT
CTGTGAACAATGCTAACTTTCCAAACCTTTAGAGTGAGTGAGAGCTGTAACTGATTT
ATCTCCTTTCCCATTGTTTAGGGGTCACCTCCAGGAATCGGACCTTGCTATGAGAAC
CACGG (SEQ ID NO: 62)
2. GGSG linker: coding sequence for GGSG linker in-frame with upstream mouse Tmprss2
coding sequence.
AGGTGGAAGCGGA (SEQ ID NO: 63)
3. P2A: porcine teschovirus 2A self-cleaving peptide sequence.
GCTACTAACTTCAGCCTGCTGAAGCAGGCTGGAGACGTGGAGGAGAACCCTGGACC
C (SEQ ID NO: 64)
4. hTMPRSS2 CDS: human TMPRSS2 cDNA coding sequence with SV40 intron inserted
for enhanced expression
ATGGCTTTGAACTCAGTAAGTTTAGTCTTTTTGTCTTTTATTTCttGTCCCGGATCCGGT
GGTGGTGCAAATCAAAGAACTGCTCCTCAGTGGATtTTGCCTTTACTTCTAGGGGTCA
CCACCAGCTATTGGACCTTACTATGAAAACCATGGATACCAACCGGAAAACCCCTAT
CCCGCACAGCCCACTGTGGTCCCCACTGTCTACGAGGTGCATCCGGCTCAGTACTAC
CCGTCCCCCGTGCCCCAGTACGCCCCGAGGGTCCTGACGCAGGCTTCCAACCCCGTC
GTCTGCACGCAGCCCAAATCCCCATCCGGGACAGTGTGCACCTCAAAGACTAAGAA
AGCACTGTGCATCACCTTGACCCTGGGGACCTTCCTCGTGGGAGCTGCGCTGGCCGC
TGGCCTACTCTGGAAGTTCATGGGCAGCAAGTGCTCCAACTCTGGGATAGAGTGCGA
CTCCTCAGGTACCTGCATCAACCCCTCTAACTGGTGTGATGGCGTGTCACACTGCCC
CGGCGGGGAGGACGAGAATCGGTGTGTTCGCCTCTACGGACCAAACTTCATCCTTCA
GGTGTACTCATCTCAGAGGAAGTCCTGGCACCCTGTGTGCCAAGACGACTGGAACG
AGAACTACGGGCGGGCGGCCTGCAGGGACATGGGCTATAAGAATAATTTTTACTCT
AGCCAAGGAATAGTGGATGACAGCGGATCCACCAGCTTTATGAAACTGAACACAAG
TGCCGGCAATGTCGATATCTATAAAAAACTGTACCACAGTGATGCCTGTTCTTCAAA
AGCAGTGGTTTCTTTACGCTGTATAGCCTGCGGGGTCAACTTGAACTCAAGCCGCCA
GAGCAGGATTGTGGGCGGCGAGAGCGCGCTCCCGGGGGCCTGGCCCTGGCAGGTCA
GCCTGCACGTCCAGAACGTCCACGTGTGCGGAGGCTCCATCATCACCCCCGAGTGG
ATCGTGACAGCCGCCCACTGCGTGGAAAAACCTCTTAACAATCCATGGCATTGGAC
GGCATTTGCGGGGATTTTGAGACAATCTTTCATGTTCTATGGAGCCGGATACCAAGT
AGAAAAAGTGATTTCTCATCCAAATTATGACTCCAAGACCAAGAACAATGACATTG
CGCTGATGAAGCTGCAGAAGCCTCTGACTTTCAACGACCTAGTGAAACCAGTGTGTC
TGCCCAACCCAGGCATGATGCTGCAGCCAGAACAGCTCTGCTGGATTTCCGGGTGG
GGGGCCACCGAGGAGAAAGGGAAGACCTCAGAAGTGCTGAACGCTGCCAAGGTGC
TTCTCATTGAGACACAGAGATGCAACAGCAGATATGTCTATGACAACCTGATCACAC
CAGCCATGATCTGTGCCGGCTTCCTGCAGGGGAACGTCGATTCTTGCCAGGGTGACA
GTGGAGGGCCTCTGGTCACTTCGAAGAACAATATCTGGTGGCTGATAGGGGATACA
AGCTGGGGTTCTGGCTGTGCCAAAGCTTACAGACCAGGAGTGTACGGGAATGTGAT
GGTATTCACGGACTGGATTTATCGACAAATGAGGGCAGACGGCTAA (SEQ ID NO: 65)
5. hTMPRSS2 3′UTR: 1837 bp human TMPRSS2 3′UTR
TCCACATGGTCTTCGTCCTTGACGTCGTTTTACAAGAAAACAATGGGGCTGGTTTTG
CTTCCCCGTGCATGATTTACTCTTAGAGATGATTCAGAGGTCACTTCATTTTTATTAA
ACAGTGAACTTGTCTGGCTTTGGCACTCTCTGCCATTCTGTGCAGGCTGCAGTGGCTC
CCCTGCCCAGCCTGCTCTCCCTAACCCCTTGTCCGCAAGGGGTGATGGCCGGCTGGT
TGTGGGCACTGGCGGTCAAGTGTGGAGGAGAGGGGTGGAGGCTGCCCCATTGAGAT
CTTCCTGCTGAGTCCTTTCCAGGGGCCAATTTTGGATGAGCATGGAGCTGTCACCTCT
CAGCTGCTGGATGACTTGAGATGAAAAAGGAGAGACATGGAAAGGGAGACAGCCA
GGTGGCACCTGCAGCGGCTGCCCTCTGGGGCCACTTGGTAGTGTCCCCAGCCTACCT
CTCCACAAGGGGATTTTGCTGATGGGTTCTTAGAGCCTTAGCAGCCCTGGATGGTGG
CCAGAAATAAAGGGACCAGCCCTTCATGGGTGGTGACGTGGTAGTCACTTGTAAGG
GGAACAGAAACATTTTTGTTCTTATGGGGTGAGAATATAGACAGTGCCCTTGGTGCG
AGGGAAGCAATTGAAAAGGAACTTGCCCTGAGCACTCCTGGTGCAGGTCTCCACCT
GCACATTGGGTGGGGCTCCTGGGAGGGAGACTCAGCCTTCCTCCTCATCCTCCCTGA
CCCTGCTCCTAGCACCCTGGAGAGTGCACATGCCCCTTGGTCCTGGCAGGGCGCCAA
GTCTGGCACCATGTTGGCCTCTTCAGGCCTGCTAGTCACTGGAAATTGAGGTCCATG
GGGGAAATCAAGGATGCTCAGTTTAAGGTACACTGTTTCCATGTTATGTTTCTACAC
ATTGCTACCTCAGTGCTCCTGGAAACTTAGCTTTTGATGTCTCCAAGTAGTCCACCTT
CATTTAACTCTTTGAAACTGTATCATCTTTGCCAAGTAAGAGTGGTGGCCTATTTCAG
CTGCTTTGACAAAATGACTGGCTCCTGACTTAACGTTCTATAAATGAATGTGCTGAA
GCAAAGTGCCCATGGTGGCGGCGAAGAAGAGAAAGATGTGTTTTGTTTTGGACTCTC
TGTGGTCCCTTCCAATGCTGTGGGTTTCCAACCAGGGGAAGGGTCCCTTTTGCATTG
CCAAGTGCCATAACCATGAGCACTACTCTACCATGGTTCTGCCTCCTGGCCAAGCAG
GCTGGTTTGCAAGAATGAAATGAATGATTCTACAGCTAGGACTTAACCTTGAAATGG
AAAGTCATGCAATCCCATTTGCAGGATCTGTCTGTGCACATGCCTCTGTAGAGAGCA
GCATTCCCAGGGACCTTGGAAACAGTTGGCACTGTAAGGTGCTTGCTCCCCAAGACA
CATCCTAAAAGGTGTTGTAATGGTGAAAACGTCTTCCTTCTTTATTGCCCCTTCTTAT
TTATGTGAACAACTGTTTGTCTTTTTTTGTATCTTTTTTAAACTGTAAAGTTCAATTGT
GAAAATGAATATCATGCAAATAAATTATGCAATTTTTTTTTCAAAGTAACTACTGCA
TCTTTGAAGTTCTGCCTGGTGAGTAGGACCAGCCTCCATTTCCTTATAAGGGGGTGA
TGTTGAGGCTGCTGGTCAGAGGACCAAAGGTGAGGCAAGGCCAGACTTGGTGCTCC
TGTGGTTGGTGCCCTCAGTTCCTGCAGCCTGTCCTGTTGGAGAGGTCCCTCAAATGA
CTCCTTCTTATTATTCTATTAGTCTGTTTCCATGCTCCTAATAAAGACATACCCAAGA
CTGCAATTTA (SEQ ID NO: 66)
6. hTMPRSS2 3′: 112 bp human intergenic sequence immediately 3′ of TMPRSS2
polyadenylation region
CAAAAGAAAGAAGTTTATTGGATTTACAATTCCACATGGCTGGGGAGGCCTCACAA
TCATGGCAGAAAGCAAGGAAGAGCAAATCACATCTTACATGGATGGCAGCAGGCAG
(SEQ ID NO: 67)
7. Tmprss2-gRNA5 Cassette: “Active genetics” cassette comprised of a mouse U6 promoter-
driven Tmprss2-gRNA5 guide RNA with 2xPolIII terminator, all in reverse orientation
relative to Tmprss2 gene
AAAAAAAGTGCCTAGCAAAAAAGCACCGACTCGGTGCCACTTTTTCAAGTTGATAA
CGGACTAGCCTTATTTTAACTTGCTATTTCTAGCTCTAAAACCGGGTATCAGTCTGAG
CACACAAACAAGGCTTTTCTCCAAGGGATATTTATAGTCTCAAAACACACAATTACT
TTACAGTTAGGGTGAGTTTCCTTTTGTGCTGTTTTTTAAAATAATAATTTAGTATTTG
TATCTCTTATA (SEQ ID NO: 68)
8. Splice-Trap FRT Cassette: splice acceptor and SV40 polyadenylation sequence followed
by a Flp Recombinase Target (FRT) site and 3-frame ATG short open reading frames. This
cassette is designed to trap and truncate any mouse Tmprss2 isoforms that may splice past
the hTMPRSS2 cassette. The FRT site allows Flp-mediated collapse of targeted tandem
integration events if required.
AATATTTTTTTCCCTCTCTAGCTTTACAATAATTTTCCCAGAACTTGTTTATTGCAGCT
TATAATGGTTACAAATAAAGCAATAGCATCACAAATTTCACAAATAAAGCATTTTTT
TCACTGCATTCTAGTTGTGGTTTGTCCAAACTCATCAATGTATCTTATCATGTCTGGA
TGAAGTTCCTATTCCGAAGTTCCTATTCTCTAGAAAGTATAGGAACTTCTGCACCAT
GGTATCACCATGCACTCACCATGGGAGATTGCGCCAACAGTATTACCACTGCCTAGC
GCGTAGTTAGGTGTAATCGGCTGAACTACCAGCGGCCG (SEQ ID NO: 69)
9. Right Homology Arm: 1352 bp 3′ homology arm encompassing sequences immediately 3′
of the Tmprss2-crRNA_5 Cas9 cut site
TATCAGTCTGAGCACATCTGTCCTCCGAGACCACCAGTGGCTCCCAATGGCTACAAC
TTGTATCCAGCCCAGTACTACCCATCTCCAGTGCCTCAGTATGCTCCGAGGATTACA
ACGCAAGCCTCAACATCTGTCATCCACACACATCCCAAGTCCTCAGGAGCACTGTGC
ACCTCAAGTAAGACTCCTTAGCATTGTATCACAAGGGACTACAGCTTCTGGATCTGC
CTTCTCTTGTGGTCTCTTATTTCTCCTACCTTGTCAATAACACTGCACTGCCAACAGG
GCCAATCTTGTCTTACCTGCCTTCCACATTGGCTGGTCAGAGCCATGGGCTCTGCCTC
ATCTCTCTTTGGCAGGGAGAGACCTTCCTCTCCCCTTGGCCTTTGGTGCAGCACCACT
GTGTGCTTGCCCAGTTCAGCTGGACTGTTAGGGCATGGTTTCTCAGTCCCTTCTTGCC
TCTGTGCTTATGGATGTGTTGTCTTGAATCAACAGTAAGCACTCAAGGTTGGGAGTG
AACTGTCTTAGATGTTATTGATGTGCTCAGAACACAGAGGGCTCAGGGTTAAGATAC
CTCCTGTGACTAGCCAGAGATCTGTTGGAGCTCTCACCTCTGCCAGTTCTTGCCCTAG
AAGAAGAAGGGTCTTCCCCACAAGGGGGATTTCTCCCATTCCAGGGCCTGCCCATAT
ACTTTCTAGGGGAGCCAGGCTGCCTCTCCACGGCAAAATTACTCTCCCAGCCCTGGA
TGTATCATGTCTTCCCCATAGCCCCTTACCCCCCCCCCCCCCATAGCCCCAGAACAGT
GCTGAAGGTTGAATATGAAAGCCGAATCTCTTTGCCCTCTCCTGTGGCCCTTTCATTT
GATGATTATTCTCACCATTAGATTCCTTACCTCTTATGTCCCTCTACCTTTTAATTCTG
GCTGTTCTCAGTGTTGCCTCTCTGGTGTCAAGGACACCAGGGACTCTATTGATAAGC
TACTTTATATATTTATTTTTTAAAACTGAGCTTGTTTGTATTACATATGTGCCAGTGC
CTATGGAGGCCAGAAGGTGTTGGATCCTCTAGAGCTGGAGTCCAAGGTATTTTCGGT
GGTTGTAAACACTCAACATGAATGCTAGGAACCAAATTCGGATCCTCTGGAAGAGC
AGTAGATGTTCCTAACTGATGGCTATTTCTCCAGCTCCACACCAGCTTTCTAGCAGA
GTTGCTGCTTTATGAGATGAGCACCTGTCTGTGTGTTTCATGTATCGTTCAAGGTTGA
TACAAAAGTGAATCCTGTGGGCCACCCATACTTCATCAGCAGTCATGTGATAGACCT
TAGGCCTCAGGAATCTGTTTGTCCCACTTGG (SEQ ID NO: 70)
pSYNB028_Ace2_NEO_BalbC (SEQ ID NO: 2)
1. Left Homology Arm: 1134 bp 5′ homology region encompassing sequences immediately
5′ of the Ace2-crRNA_32 Cas9 cut site
GAGTTCTCAGCTGAGTTGTAAGCAGGTAAGTGAAAGGGAAAGAGGCACCTGATAAA
GTCAGCTGTAGGACTAGAGTTTAGCATGTCTCCTGAGAAATAGAAAATGACTGCTTG
AAACTTTACCAAAGCCACACAGGAAGCATCAAACTCCCTATGGAGTGGAGAAGAGT
CTTATAATTTTTTAAATGGGCAGAGAAATGAATTTATTTTTAATTTTTAGAGACAGG
GTTTCTTTGTATAGCTCTAGCTGTCTTTGATTGGTAGACAAAGCTGTCCTCAAACTCA
GAGATCTTCCTTCCTTTGTCTCCTGAGTGCTGGGATTAAAGGCATGGACCACCACTG
CCCTGCCCCATTCTCTCCATTAATTTTAAGTGAATGCTTGCAAAAGCTCACTTCTTTG
GTGAACAGCTTCCTTTACAAATAAGTACCTTTGCCTTCGTTTTTATAGGATTCTTAAA
AAGAAAAAAAAGATTCAGCCAGGTGGTTGTGGTGCACACCTTTAATCCCAGCAGTC
AGGAGGCAGAGGAAAGCAGATCTCTTGAGTTTGAGGCTAGCCTAGTCTACAGAGGG
AGTTCCAGGACAGCCAAGGCTACAGAGAGGAACTGTCTAAAAACACCAAGAAAGA
GAGAAAGGAGAGAGGGAGAGGATGGATAGCTTATTGATAGAATTGTCAGAAAAGG
CTATAAGTTCCAATATGTGTCCCATGATTTCTAAGTCTAGCCCTTTCTGTTATAGTAA
AATCATAGTACACCCTCCTCCTCCAGTGTATCTTTAACAGCTTTTAAGGAACATATTA
ACTAAATGTCCAGGTTTTGATTTGGCCATAAAATGTTAGCAAAGCTAAGGTTTTCTA
GGATTAATGAATAACATGTCTTTATTTAGTTTACTTAAAAAAATCATTCTAAAATATC
TGTTTACATATCTGTCCTCTCCAGGATTAACTTCATATTGGTCCAGCAGCTTGTTTAC
TGTTCTCTTCTGTTTCTTCTTCTGCTTTTTTTTTCTTCTCTTCTCAGTGCCCAACCCAAG
TTCAAAGGCTGATGAGAGAGAAAAACTCATGAAGAGATTTTACTCTAGGGAAAGTT
GCTCAGTGGATGGGATCTTGGCGCACGGGGAAAGATGTCCAGCTCCTCCT (SEQ ID
NO: 53)
2. Mouse Signal Peptide: signal peptide coding sequence (translation produces amino acid
sequence WLLLSLVAVTTA (SEQ ID NO: 54)) in-frame with first 5 amino acids of mouse Ace2
GGCTTCTGCTTTCTCTGGTGGCAGTGACAACAGCA (SEQ ID NO: 55)
3. hACE2 CDS: human ACE2 cDNA coding sequence with SV40 intron inserted for
enhanced expression
CAGTCCACCATTGAGGAACAGGCCAAGACATTTTTGGACAAGTTTAACCACGAAGC
CGAAGACCTGTTCTATCAAAGTTCACTTGCTTCTTGGAATTATAACACCAATATTACT
GAAGAGAATGTCCAAAACATGGTAAGTTTAGTCTTTTTGTCTTTTATTTCttGTCCCGG
ATCCGGTGGTGGTGCAAATCAAAGAACTGCTCCTCAGTGGATtTTGCCTTTACTTCTA
GAATAATGCTGGGGACAAATGGTCTGCCTTTTTAAAGGAACAGTCCACACTTGCCCA
AATGTATCCACTACAAGAAATTCAGAATCTCACAGTCAAGCTTCAGCTGCAGGCTCT
TCAGCAAAATGGGTCTTCAGTGCTCTCAGAAGACAAGAGCAAACGGTTGAACACAA
TTCTAAATACAATGAGCACCATCTACAGTACTGGAAAAGTTTGTAACCCAGATAATC
CACAAGAATGCTTATTACTTGAACCAGGTTTGAATGAAATAATGGCAAACAGTTTAG
ACTACAATGAGAGGCTCTGGGCTTGGGAAAGCTGGAGATCTGAGGTCGGCAAGCAG
CTGAGGCCATTATATGAAGAGTATGTGGTCTTGAAAAATGAGATGGCAAGAGCAAA
TCATTATGAGGACTATGGGGATTATTGGAGAGGAGACTATGAAGTAAATGGGGTAG
ATGGCTATGACTACAGCCGCGGCCAGTTGATTGAAGATGTGGAACATACCTTTGAAG
AGATTAAACCATTATATGAACATCTTCATGCCTATGTGAGGGCAAAGTTGATGAATG
CCTATCCTTCCTATATCAGTCCAATTGGATGCCTCCCTGCTCATTTGCTTGGTGATAT
GTGGGGTAGATTTTGGACAAATCTGTACTCTTTGACAGTTCCCTTTGGACAGAAACC
AAACATAGATGTTACTGATGCAATGGTGGACCAGGCCTGGGATGCACAGAGAATAT
TCAAGGAGGCCGAGAAGTTCTTTGTATCTGTTGGTCTTCCTAATATGACTCAAGGAT
TCTGGGAAAATTCCATGCTAACGGACCCAGGAAATGTTCAGAAAGCAGTCTGCCAT
CCCACAGCTTGGGACCTGGGGAAGGGCGACTTCAGGATCCTTATGTGCACAAAGGT
GACAATGGACGACTTCCTGACAGCTCATCATGAGATGGGGCATATCCAGTATGATAT
GGCATATGCTGCACAACCTTTTCTGCTAAGAAATGGAGCTAATGAAGGATTCCATGA
AGCTGTTGGGGAAATCATGTCACTTTCTGCAGCCACACCTAAGCATTTAAAATCCAT
TGGTCTTCTGTCACCCGATTTTCAAGAAGACAATGAAACAGAAATAAACTTCCTGCT
CAAACAAGCACTCACGATTGTTGGGACTCTGCCATTTACTTACATGTTAGAGAAGTG
GAGGTGGATGGTCTTTAAAGGGGAAATTCCCAAAGACCAGTGGATGAAAAAGTGGT
GGGAGATGAAGCGAGAGATAGTTGGGGTGGTGGAACCTGTGCCCCATGATGAAACA
TACTGTGACCCCGCATCTCTGTTCCATGTTTCTAATGATTACTCATTCATTCGATATT
ACACAAGGACCCTTTACCAATTCCAGTTTCAAGAAGCACTTTGTCAAGCAGCTAAAC
ATGAAGGCCCTCTGCACAAATGTGACATCTCAAACTCTACAGAAGCTGGACAGAAA
CTGTTCAATATGCTGAGGCTTGGAAAATCAGAACCCTGGACCCTAGCATTGGAAAAT
GTTGTAGGAGCAAAGAACATGAATGTAAGGCCACTGCTCAACTACTTTGAGCCCTTA
TTTACCTGGCTGAAAGACCAGAACAAGAATTCTTTTGTGGGATGGAGTACCGACTGG
AGTCCATATGCAGACCAAAGCATCAAAGTGAGGATAAGCCTAAAATCAGCTCTTGG
AGATAAAGCATATGAATGGAACGACAATGAAATGTACCTGTTCCGATCATCTGTTGC
ATATGCTATGAGGCAGTACTTTTTAAAAGTAAAAAATCAGATGATTCTTTTTGGGGA
GGAGGATGTGCGAGTGGCTAATTTGAAACCAAGAATCTCCTTTAATTTCTTTGTCAC
TGCACCTAAAAATGTGTCTGATATCATTCCTAGAACTGAAGTTGAAAAGGCCATCAG
GATGTCCCGGAGCCGTATCAATGATGCTTTCCGTCTGAATGACAACAGCCTAGAGTT
TCTGGGGATACAGCCAACACTTGGACCTCCTAACCAGCCCCCTGTTTCCATATGGCT
GATTGTTTTTGGAGTTGTGATGGGAGTGATAGTGGTTGGCATTGTCATCCTGATCTTC
ACTGGGATCAGAGATCGGAAGAAGAAAAATAAAGCAAGAAGTGGAGAAAATCCTT
ATGCCTCCATCGATATTAGCAAAGGAGAAAATAATCCAGGATTCCAAAACACTGAT
GATGTTCAGACCTCCTTTTAG (SEQ ID NO: 56)
4. hACE2 3′UTR: 872 bp human ACE2 3 ′UTR
AAAAATCTATGTTTTTCCTCTTGAGGTGATTTTGTTGTATGTAAATGTTAATTTCATG
GTATAGAAAATATAAGATGATAAAGATATCATTAAATGTCAAAACTATGACTCTGTT
CAGAAAAAAAATTGTCCAAAGACAACATGGCCAAGGAGAGAGCATCTTCATTGACA
TTGCTTTCAGTATTTATTTCTGTCTCTGGATTTGACTTCTGTTCTGTTTCTTAATAAGG
ATTTTGTATTAGAGTATATTAGGGAAAGTGTGTATTTGGTCTCACAGGCTGTTCAGG
GATAATCTAAATGTAAATGTCTGTTGAATTTCTGAAGTTGAAAACAAGGATATATCA
TTGGAGCAAGTGTTGGATCTTGTATGGAATATGGATGGATCACTTGTAAGGACAGTG
CCTGGGAACTGGTGTAGCTGCAAGGATTGAGAATGGCATGCATTAGCTCACTTTCAT
TTAATCCATTGTCAAGGATGACATGCTTTCTTCACAGTAACTCAGTTCAAGTACTATG
GTGATTTGCCTACAGTGATGTTTGGAATCGATCATGCTTTCTTCAAGGTGACAGGTCT
AAAGAGAGAAGAATCCAGGGAACAGGTAGAGGACATTGCTTTTTCACTTCCAAGGT
GCTTGATCAACATCTCCCTGACAACACAAAACTAGAGCCAGGGGCCTCCGTGAACT
CCCAGAGCATGCCTGATAGAAACTCATTTCTACTGTTCTCTAACTGTGGAGTGAATG
GAAATTCCAACTGTATGTTCACCCTCTGAAGTGGGTACCCAGTCTCTTAAATCTTTTG
TATTTGCTCACAGTGTTTGAGCAGTGCTGAGCACAAAGCAGACACTCAATAAATGCT
AGATTTACACACTC (SEQ ID NO: 57)
5. hACE2 3′: 100 bp human intergenic sequence immediately 3′ of ACE2 polyadenylation
region
CTTGTGCTTACTTATGTGCTGGGGCTTCTTTACGTTTTGTCTGCTTTTCAGTTCTATGA
ACAAAGTTATCATCTGAGTGTCTGGGGCTCCTGGCCTTCCA (SEQ ID NO: 58)
6. Ace2-gRNA32 Cassette: “Active genetics” cassette comprised of a mouse U6 promoter-
driven Ace2-gRNA32 guide RNA with 2xPolIII terminator, all in reverse orientation relative
to Ace2
AAAAAAAGTGCCTAGCAAAAAAGCACCGACTCGGTGCCACTTTTTCAAGTTGATAA
CGGACTAGCCTTATTTTAACTTGCTATTTCTAGCTCTAAAACCCTGGCTCCTTCTCAG
CCTTCAAACAAGGCTTTTCTCCAAGGGATATTTATAGTCTCAAAACACACAATTACT
TTACAGTTAGGGTGAGTTTCCTTTTGTGCTGTTTTTTAAAATAATAATTTAGTATTTG
TATCTCTTATA (SEQ ID NO: 59)
7. SA-SV40 poly(A) signal: splice acceptor and SV40 polyadenylation sequence designed to
trap and truncate any mouse Ace2 isoforms that may splice past the hACE2 cassette.
AATATTTTTTTCCCTCTCTAGCTTTACAATAATTTTCCCAGAACTTGTTTATTGCAGCT
TATAATGGTTACAAATAAAGCAATAGCATCACAAATTTCACAAATAAAGCATTTTTT
TCACTGCATTCTAGTTGTGGTTTGTCCAAACTCATCAATGTATCTTATCATGTCTGGA
T (SEQ ID NO: 71)
8. FRT-PGK-EM7-Neo-bGHpA-FRT: Neomycin resistance gene driven by the mouse PGK
promoter and with a bovine growth hormone polyadenylation sequence. The FRT sites allow Flp-
mediated removal of the neomycin cassette from targeted embryonic stem cell clones as well
as collapse of targeted tandem integration events if required.
GAAGTTCCTATTCCGAAGTTCCTATTCTCTAGAAAGTATAGGAACTTCAGGTCTGAA
GAGGAGTTTACGTCCAGCCAAGCTAGCTTGGCTGCAGGTCGTCGAAATTCTACCGGG
TAGGGGAGGCGCTTTTCCCAAGGCAGTCTGGAGCATGCGCTTTAGCAGCCCCGCTGG
GCACTTGGCGCTACACAAGTGGCCTCTGGCCTCGCACACATTCCACATCCACCGGTA
GGCGCCAACCGGCTCCGTTCTTTGGTGGCCCCTTCGCGCCACCTTCTACTCCTCCCCT
AGTCAGGAAGTTCCCCCCCGCCCCGCAGCTCGCGTCGTGCAGGACGTGACAAATGG
AAGTAGCACGTCTCACTAGTCTCGTGCAGATGGACAGCACCGCTGAGCAATGGAAG
CGGGTAGGCCTTTGGGGCAGCGGCCAATAGCAGCTTTGCTCCTTCGCTTTCTGGGCT
CAGAGGCTGGGAAGGGGTGGGTCCGGGGGCGGGCTCAGGGGCGGGCTCAGGGGCG
GGGCGGGCGCCCGAAGGTCCTCCGGAGGCCCGGCATTCTGCACGCTTCAAAAGCGC
ACGTCTGCCGCGCTGTTCTCCTCTTCCTCATCTCCGGGCCTTTCGACCTGCAGCCTGT
TGACAATTAATCATCGGCATAGTATATCGGCATAGTATAATACGACAAGGTGAGGA
ACTAAACCATGGGATCGGCCATTGAACAAGATGGATTGCACGCAGGTTCTCCGGCC
GCTTGGGTGGAGAGGCTATTCGGCTATGACTGGGCACAACAGACAATCGGCTGCTCT
GATGCCGCCGTGTTCCGGCTGTCAGCGCAGGGGCGCCCGGTTCTTTTTGTCAAGACC
GACCTGTCCGGTGCCCTGAATGAACTGCAGGACGAGGCAGCGCGGCTATCGTGGCT
GGCCACGACGGGCGTTCCTTGCGCAGCTGTGCTCGACGTTGTCACTGAAGCGGGAA
GGGACTGGCTGCTATTGGGCGAAGTGCCGGGGCAGGATCTCCTGTCATCTCACCTTG
CTCCTGCCGAGAAAGTATCCATCATGGCTGATGCAATGCGGCGGCTGCATACGCTTG
ATCCGGCTACCTGCCCATTCGACCACCAAGCGAAACATCGCATCGAGCGAGCACGT
ACTCGGATGGAAGCCGGTCTTGTCGATCAGGATGATCTGGACGAAGAGCATCAGGG
GCTCGCGCCAGCCGAACTGTTCGCCAGGCTCAAGGCGCGCATGCCCGACGGCGATG
ATCTCGTCGTGACCCATGGCGATGCCTGCTTGCCGAATATCATGGTGGAAAATGGCC
GCTTTTCTGGATTCATCGACTGTGGCCGGCTGGGTGTGGCGGACCGCTATCAGGACA
TAGCGTTGGCTACCCGTGATATTGCTGAAGAGCTTGGCGGCGAATGGGCTGACCGCT
TCCTCGTGCTTTACGGTATCGCCGCTCCCGATTCGCAGCGCATCGCCTTCTATCGCCT
TCTTGACGAGTTCTTCTGAGGGGATCAATTCTCTAGAGCTCGCTGATCAGCCTCGAC
TGTGCCTTCTAGTTGCCAGCCATCTGTTGTTTGCCCCTCCCCCGTGCCTTCCTTGACC
CTGGAAGGTGCCACTCCCACTGTCCTTTCCTAATAAAATGAGGAAATTGCATCGCAT
TGTCTGAGTAGGTGTCATTCTATTCTGGGGGGTGGGGTGGGGCAGGACAGCAAGGG
GGAGGATTGGGAAGACAATAGCAGGCATGCTGGGGATGCGGTGGGCTCTATGGCTT
CTGAGGCGGAAAGAACCAGCTGGGGCTCGACTAGAGCTTGCGGAACCCTTCGAAGT
TCCTATTCTCTAGAAAGTATAGGAACTTCTGCACCATGGTATCACCATGCACTCACC
ATGGGAGATTGCGCCAACAGTATTACCACTGCCTAGCGCGTAGTTAGGTGTAATCGG
CTGAACTACCAGCGGCCG (SEQ ID NO: 72)
9. Right Homology Arm: 1535 bp 3′ homology arm encompassing sequences immediately 3′
of the Ace2-crRNA_32 Cas9 cut site
CCTTCTCAGCCTTGTTGCTGTTACTACTGCTCAGTCCCTCACCGAGGAAAATGCCAA
GACATTTTTAAACAACTTTAATCAGGAAGCTGAAGACCTGTCTTATCAAAGTTCACT
TGCTTCTTGGAATTATAATACTAACATTACTGAAGAAAATGCCCAAAAGATGGTAAG
TTCTTGAGGCTACCCAGGGGGTTATTGATTGCTTCTTAAAGATCAGAATTACTGCCT
ATAAAACTGGATAAGGAAATCATAGAGATCTCTCAAGTGTGAGGATGAGTGACTGC
CTCTGTAGCTCTGATCCTAGTCTCCCAGATGGCTAAATTCAATTGACCTTAGAGTTCA
TCTGGAAAATTGTTATGAATGAATTATTTGCCCAGATTCCAAAGATGAGTGAAAATG
TTTAATAAAGTTGCCATCACTATTCTCATTATATTTGGTATGTAAAGCATTCATGGAA
ATGTTCTAAGTCGTTATTGAGCCAATAATTTTCTTTAGCTTATAATGCCAACAGGTCT
ATCCGAGAACTACAAATGACATATTAACTGAAAAATGCAACTGGGGTTTACTGAAG
GCAGCAGCTTAGTAATTAAGGTAACCATGGCTTAGGTGAAACTGGACCTGGGAATT
CCTTCTTTCATTGACACAGAGCTCTGAGGAATTTCCAAAGGTCACAGAAGAAAAGCT
ATAATTAAACTAGTCCCAAAAAATCTCAGCCTACTCTGGGAAAGCAGCATATTTTGT
TTGACAAGTGCAAGGACTTAGAACTTTTTTTTTTCTCACTGATCCTGAAGTGCCTTTT
AAGTATAGTTAAGTGGTGGAAAATTGAGCAACTATTTAAGAAAAGACTCTTTTTTTT
CTTCTTCCAGCAATGCTTTCCTTCAAAACGGTAGCTTCAAAACTTCCTGTCTTTTAAA
TGATCAGGGGGCTGTGTGTTTAAATTATTGCCATTCATAGAACAGAGTGGGTCTGAG
GATGCCTGTTTCCTTTGAAATTCTATGCCCCCTCCCAGTTTTCTAAAATTTAAGAAAC
CACAGAGACTTTGACAATGTAGTTGCCAAATGAGTTGCTTTTAACTGCTCTAATAGT
TTGGTCTTACCGGTGTTGTTTTTAGTGTAACTTTTTTTCCCCTCAGTTTTTATTTTATTT
CTGTTGGAGATCACCAACGTGTACTGTAGATTTTCTGAGCTGAATGGTTCAAGGTGA
GACTAATATTATACTTGAAAGCCTAGCCTGGAGCTGACATCCAGATCCTGGCTGTAC
CACTTTTTAGCTGGGTAAACTTGAATTCATTAAATGGACAGTCTGGGTCTTAGTTACT
TTTTGTTTTAAGATGTATTTTAATTTTAAAACTATGTGTATGTGTGTGTGCTCCCGAA
TATAAGTATAGATGCCCATGAAGGTCAGAGATATTGGAACCCCCAGGAGTGGTGCA
GCAGGCTATTGTGTGCCACCAGACCTAGATGCTGGCAACTTGCAACCTCTGCAAGGG
AAGTACATATCCTTAACCATCGAGCCACCTTTCTACCCCAGTCTTA (SEQ ID NO: 61)
pSYNB029_BalbC_Tmprss2_NEO Humanizing Construct (SEQ ID NO: 4)
1. Left Homology Arm: 1503 bp 5′ homology region encompassing sequences immediately
5′ of the Tmprss2-crRNA_5 Cas9 cut site
GTCATCCAGGACAAAGTGCTAGGTGTATCTTGTTCTTTCCTGCTGGAGAGGGGAACA
CATCCTTAGACGGTTTGGTTCTGGGACAGACACTCACCTGGTTTCTCAATCTGGAAT
GCTCTGTTAATATGGGAATAGCTTTCGCTGTCCCACCCTGAGGTTTCAAGTTATAATC
TTTGGACCTTTGAAGGAGAGTGCTAGTGAGTTATTAAGCACAGATGATTAACCTTGG
AGTGTTGGTATTGCCTTAAAATTCTCAACCTTTAGGCCAGCAAGACAGCTCCATAAG
GTAAGAGATGACCTGCATTCAATCTTTGTGGCCACAGGGTGGAAGGAGACAACTGG
CTCTCACAGGTTGTCCTATGATCTCTGGTTACACACACGTGCAACACACACAATAAA
TACATGAGCTTTTAAAAGTCTGAGCATCCAGTGGTCCCTGCTGAGGATATCTGCTTC
CAGAAGATGATTTTTCTGTCATTCCTGAGGCCCATGAGAGAACCTGAGCAGAAAGA
GAGCTGGAGAGGTTTGGGGTAAGAACCATTAGACATGCATAGGGAGACCTCTCTGG
GAGAGGTCCAGCTACTGAGAGTAGGGAGAGTGGAAATAACAATGCCAACCTGTGAA
CTTGCTACTGAACCTGACAACTGCTGGGCCATACTAGGAGTACAGTCTGGGGAAGGT
CACGTGAATGGACTGAGTGAAAGGATTGATACCCAAGAAGTGGATCCTGCCCTTTTT
ACATTCACACAGCACTTACTTTGACCTCCAAGTTCCCCTGAAAGGGCACCTACACCC
TCAGCACCAAAATGCATACACTCTATAGGGATGTTCACACTGAGCTAACACCATCCT
GGTGAGAACAGATGGGTAAAGGACTACCAACTACAGCTGCATGAGAGAAGGACCCT
GGAAGAGAATGGCCACAGGAGTCTGCATTGTGGGTCCCTTCTAGCAGTTTCTCAGCT
CAGCTGAAAAGTGTATTTACTACACCCAAGGTTGCACACATTATCTGAATATGAGGT
CCCTTTAGCAGGTGAGTTCTCAGATATGAGCTATATCCTGAACCTTGACATTAACCT
GGCTCAAAATTTCAGACCTCAAGGCTCAGGGGGTGATTTAGAGGAGGAACCAGAGA
ATGCGACTCTAACTGTGTGACTTTTGACTCCAAACTTGTATGTATGTTTTCTACAGAA
TCTTAGTTATCGGTCACCTTTTCTTTATTTCAAAGAACAAATAATGAAAAAAAAATG
GTCCAGGAAACCAAAGCTAATGAAACACAGATAGTTGAGCGTTCTAGTTAGTGCAG
AAACAAGAGTCTAGTCATGTTAAGCAAGTTGCTGTGATGACAGAGTGATTGGCAAT
CTGCTGGCTCTCTCCCTGTTTTCTGTGAACAATGCTAACTTTCCAAACCTTTAGAGTG
AGTGAGAGCTGTAACTGATTTATCTCCTTTCCCATTGTTTAGGGGTCACCTCCAGGA
ATCGGACCTTGCTATGAGAACCACGG (SEQ ID NO: 73)
2. GGSG linker: coding sequence for GGSG linker in-frame with upstream mouse Tmprss2
coding sequence.
AGGTGGAAGCGGA (SEQ ID NO: 63)
3. P2A: porcine teschovirus 2A self-cleaving peptide sequence.
GCTACTAACTTCAGCCTGCTGAAGCAGGCTGGAGACGTGGAGGAGAACCCTGGACC
C (SEQ ID NO: 64)
4. hTMPRSS2 CDS: human TMPRSS2 cDNA coding sequence with SV40 intron inserted
for enhanced expression
ATGGCTTTGAACTCAGTAAGTTTAGTCTTTTTGTCTTTTATTTCttGTCCCGGATCCGGT
GGTGGTGCAAATCAAAGAACTGCTCCTCAGTGGATtTTGCCTTTACTTCTAGGGGTCA
CCACCAGCTATTGGACCTTACTATGAAAACCATGGATACCAACCGGAAAACCCCTAT
CCCGCACAGCCCACTGTGGTCCCCACTGTCTACGAGGTGCATCCGGCTCAGTACTAC
CCGTCCCCCGTGCCCCAGTACGCCCCGAGGGTCCTGACGCAGGCTTCCAACCCCGTC
GTCTGCACGCAGCCCAAATCCCCATCCGGGACAGTGTGCACCTCAAAGACTAAGAA
AGCACTGTGCATCACCTTGACCCTGGGGACCTTCCTCGTGGGAGCTGCGCTGGCCGC
TGGCCTACTCTGGAAGTTCATGGGCAGCAAGTGCTCCAACTCTGGGATAGAGTGCGA
CTCCTCAGGTACCTGCATCAACCCCTCTAACTGGTGTGATGGCGTGTCACACTGCCC
CGGCGGGGAGGACGAGAATCGGTGTGTTCGCCTCTACGGACCAAACTTCATCCTTCA
GGTGTACTCATCTCAGAGGAAGTCCTGGCACCCTGTGTGCCAAGACGACTGGAACG
AGAACTACGGGCGGGCGGCCTGCAGGGACATGGGCTATAAGAATAATTTTTACTCT
AGCCAAGGAATAGTGGATGACAGCGGATCCACCAGCTTTATGAAACTGAACACAAG
TGCCGGCAATGTCGATATCTATAAAAAACTGTACCACAGTGATGCCTGTTCTTCAAA
AGCAGTGGTTTCTTTACGCTGTATAGCCTGCGGGGTCAACTTGAACTCAAGCCGCCA
GAGCAGGATTGTGGGCGGCGAGAGCGCGCTCCCGGGGGCCTGGCCCTGGCAGGTCA
GCCTGCACGTCCAGAACGTCCACGTGTGCGGAGGCTCCATCATCACCCCCGAGTGG
ATCGTGACAGCCGCCCACTGCGTGGAAAAACCTCTTAACAATCCATGGCATTGGAC
GGCATTTGCGGGGATTTTGAGACAATCTTTCATGTTCTATGGAGCCGGATACCAAGT
AGAAAAAGTGATTTCTCATCCAAATTATGACTCCAAGACCAAGAACAATGACATTG
CGCTGATGAAGCTGCAGAAGCCTCTGACTTTCAACGACCTAGTGAAACCAGTGTGTC
TGCCCAACCCAGGCATGATGCTGCAGCCAGAACAGCTCTGCTGGATTTCCGGGTGG
GGGGCCACCGAGGAGAAAGGGAAGACCTCAGAAGTGCTGAACGCTGCCAAGGTGC
TTCTCATTGAGACACAGAGATGCAACAGCAGATATGTCTATGACAACCTGATCACAC
CAGCCATGATCTGTGCCGGCTTCCTGCAGGGGAACGTCGATTCTTGCCAGGGTGACA
GTGGAGGGCCTCTGGTCACTTCGAAGAACAATATCTGGTGGCTGATAGGGGATACA
AGCTGGGGTTCTGGCTGTGCCAAAGCTTACAGACCAGGAGTGTACGGGAATGTGAT
GGTATTCACGGACTGGATTTATCGACAAATGAGGGCAGACGGCTAA (SEQ ID NO: 65)
5. hTMPRSS2 3′UTR: 1837 bp human TMPRSS2 3′UTR
TCCACATGGTCTTCGTCCTTGACGTCGTTTTACAAGAAAACAATGGGGCTGGTTTTG
CTTCCCCGTGCATGATTTACTCTTAGAGATGATTCAGAGGTCACTTCATTTTTATTAA
ACAGTGAACTTGTCTGGCTTTGGCACTCTCTGCCATTCTGTGCAGGCTGCAGTGGCTC
CCCTGCCCAGCCTGCTCTCCCTAACCCCTTGTCCGCAAGGGGTGATGGCCGGCTGGT
TGTGGGCACTGGCGGTCAAGTGTGGAGGAGAGGGGTGGAGGCTGCCCCATTGAGAT
CTTCCTGCTGAGTCCTTTCCAGGGGCCAATTTTGGATGAGCATGGAGCTGTCACCTCT
CAGCTGCTGGATGACTTGAGATGAAAAAGGAGAGACATGGAAAGGGAGACAGCCA
GGTGGCACCTGCAGCGGCTGCCCTCTGGGGCCACTTGGTAGTGTCCCCAGCCTACCT
CTCCACAAGGGGATTTTGCTGATGGGTTCTTAGAGCCTTAGCAGCCCTGGATGGTGG
CCAGAAATAAAGGGACCAGCCCTTCATGGGTGGTGACGTGGTAGTCACTTGTAAGG
GGAACAGAAACATTTTTGTTCTTATGGGGTGAGAATATAGACAGTGCCCTTGGTGCG
AGGGAAGCAATTGAAAAGGAACTTGCCCTGAGCACTCCTGGTGCAGGTCTCCACCT
GCACATTGGGTGGGGCTCCTGGGAGGGAGACTCAGCCTTCCTCCTCATCCTCCCTGA
CCCTGCTCCTAGCACCCTGGAGAGTGCACATGCCCCTTGGTCCTGGCAGGGCGCCAA
GTCTGGCACCATGTTGGCCTCTTCAGGCCTGCTAGTCACTGGAAATTGAGGTCCATG
GGGGAAATCAAGGATGCTCAGTTTAAGGTACACTGTTTCCATGTTATGTTTCTACAC
ATTGCTACCTCAGTGCTCCTGGAAACTTAGCTTTTGATGTCTCCAAGTAGTCCACCTT
CATTTAACTCTTTGAAACTGTATCATCTTTGCCAAGTAAGAGTGGTGGCCTATTTCAG
CTGCTTTGACAAAATGACTGGCTCCTGACTTAACGTTCTATAAATGAATGTGCTGAA
GCAAAGTGCCCATGGTGGCGGCGAAGAAGAGAAAGATGTGTTTTGTTTTGGACTCTC
TGTGGTCCCTTCCAATGCTGTGGGTTTCCAACCAGGGGAAGGGTCCCTTTTGCATTG
CCAAGTGCCATAACCATGAGCACTACTCTACCATGGTTCTGCCTCCTGGCCAAGCAG
GCTGGTTTGCAAGAATGAAATGAATGATTCTACAGCTAGGACTTAACCTTGAAATGG
AAAGTCATGCAATCCCATTTGCAGGATCTGTCTGTGCACATGCCTCTGTAGAGAGCA
GCATTCCCAGGGACCTTGGAAACAGTTGGCACTGTAAGGTGCTTGCTCCCCAAGACA
CATCCTAAAAGGTGTTGTAATGGTGAAAACGTCTTCCTTCTTTATTGCCCCTTCTTAT
TTATGTGAACAACTGTTTGTCTTTTTTTGTATCTTTTTTAAACTGTAAAGTTCAATTGT
GAAAATGAATATCATGCAAATAAATTATGCAATTTTTTTTTCAAAGTAACTACTGCA
TCTTTGAAGTTCTGCCTGGTGAGTAGGACCAGCCTCCATTTCCTTATAAGGGGGTGA
TGTTGAGGCTGCTGGTCAGAGGACCAAAGGTGAGGCAAGGCCAGACTTGGTGCTCC
TGTGGTTGGTGCCCTCAGTTCCTGCAGCCTGTCCTGTTGGAGAGGTCCCTCAAATGA
CTCCTTCTTATTATTCTATTAGTCTGTTTCCATGCTCCTAATAAAGACATACCCAAGA
CTGCAATTTA (SEQ ID NO: 66)
6. hTMPRSS2 3′: 112 bp human intergenic sequence immediately 3′ of TMPRSS2
polyadenylation region
CAAAAGAAAGAAGTTTATTGGATTTACAATTCCACATGGCTGGGGAGGCCTCACAA
TCATGGCAGAAAGCAAGGAAGAGCAAATCACATCTTACATGGATGGCAGCAGGCAG
(SEQ ID NO: 67)
7. Tmprss2-gRNA5 Cassette: “Active genetics” cassette comprised of a mouse U6 promoter-
driven Tmprss2-gRNA5 guide RNA with 2xPolIII terminator, all in reverse orientation
relative to Tmprss2 gene
AAAAAAAGTGCCTAGCAAAAAAGCACCGACTCGGTGCCACTTTTTCAAGTTGATAA
CGGACTAGCCTTATTTTAACTTGCTATTTCTAGCTCTAAAACCGGGTATCAGTCTGAG
CACACAAACAAGGCTTTTCTCCAAGGGATATTTATAGTCTCAAAACACACAATTACT
TTACAGTTAGGGTGAGTTTCCTTTTGTGCTGTTTTTTAAAATAATAATTTAGTATTTG
TATCTCTTATA (SEQ ID NO: 68)
8. SA-SV40 poly(A) signal: splice acceptor and SV40 polyadenylation sequence designed to
trap and truncate any mouse Tmprss2 isoforms that may splice past the hTMPRSS2 cassette.
AATATTTTTTTCCCTCTCTAGCTTTACAATAATTTTCCCAGAACTTGTTTATTGCAGCT
TATAATGGTTACAAATAAAGCAATAGCATCACAAATTTCACAAATAAAGCATTTTTT
TCACTGCATTCTAGTTGTGGTTTGTCCAAACTCATCAATGTATCTTATCATGTCTGGA
T (SEQ ID NO: 71)
9. FRT-PGK-EM7-Neo-bGHpA-FRT: Neomycin resistance gene driven by the mouse PGK
promoter and with a bovine growth hormone polyadenylation sequence. The FRT sites allow Flp-
mediated removal of the neomycin cassette from targeted embryonic stem cell clones as well
as collapse of targeted tandem integration events if required.
GAAGTTCCTATTCCGAAGTTCCTATTCTCTAGAAAGTATAGGAACTTCAGGTCTGAA
GAGGAGTTTACGTCCAGCCAAGCTAGCTTGGCTGCAGGTCGTCGAAATTCTACCGGG
TAGGGGAGGCGCTTTTCCCAAGGCAGTCTGGAGCATGCGCTTTAGCAGCCCCGCTGG
GCACTTGGCGCTACACAAGTGGCCTCTGGCCTCGCACACATTCCACATCCACCGGTA
GGCGCCAACCGGCTCCGTTCTTTGGTGGCCCCTTCGCGCCACCTTCTACTCCTCCCCT
AGTCAGGAAGTTCCCCCCCGCCCCGCAGCTCGCGTCGTGCAGGACGTGACAAATGG
AAGTAGCACGTCTCACTAGTCTCGTGCAGATGGACAGCACCGCTGAGCAATGGAAG
CGGGTAGGCCTTTGGGGCAGCGGCCAATAGCAGCTTTGCTCCTTCGCTTTCTGGGCT
CAGAGGCTGGGAAGGGGTGGGTCCGGGGGCGGGCTCAGGGGCGGGCTCAGGGGCG
GGGCGGGCGCCCGAAGGTCCTCCGGAGGCCCGGCATTCTGCACGCTTCAAAAGCGC
ACGTCTGCCGCGCTGTTCTCCTCTTCCTCATCTCCGGGCCTTTCGACCTGCAGCCTGT
TGACAATTAATCATCGGCATAGTATATCGGCATAGTATAATACGACAAGGTGAGGA
ACTAAACCATGGGATCGGCCATTGAACAAGATGGATTGCACGCAGGTTCTCCGGCC
GCTTGGGTGGAGAGGCTATTCGGCTATGACTGGGCACAACAGACAATCGGCTGCTCT
GATGCCGCCGTGTTCCGGCTGTCAGCGCAGGGGCGCCCGGTTCTTTTTGTCAAGACC
GACCTGTCCGGTGCCCTGAATGAACTGCAGGACGAGGCAGCGCGGCTATCGTGGCT
GGCCACGACGGGCGTTCCTTGCGCAGCTGTGCTCGACGTTGTCACTGAAGCGGGAA
GGGACTGGCTGCTATTGGGCGAAGTGCCGGGGCAGGATCTCCTGTCATCTCACCTTG
CTCCTGCCGAGAAAGTATCCATCATGGCTGATGCAATGCGGCGGCTGCATACGCTTG
ATCCGGCTACCTGCCCATTCGACCACCAAGCGAAACATCGCATCGAGCGAGCACGT
ACTCGGATGGAAGCCGGTCTTGTCGATCAGGATGATCTGGACGAAGAGCATCAGGG
GCTCGCGCCAGCCGAACTGTTCGCCAGGCTCAAGGCGCGCATGCCCGACGGCGATG
ATCTCGTCGTGACCCATGGCGATGCCTGCTTGCCGAATATCATGGTGGAAAATGGCC
GCTTTTCTGGATTCATCGACTGTGGCCGGCTGGGTGTGGCGGACCGCTATCAGGACA
TAGCGTTGGCTACCCGTGATATTGCTGAAGAGCTTGGCGGCGAATGGGCTGACCGCT
TCCTCGTGCTTTACGGTATCGCCGCTCCCGATTCGCAGCGCATCGCCTTCTATCGCCT
TCTTGACGAGTTCTTCTGAGGGGATCAATTCTCTAGAGCTCGCTGATCAGCCTCGAC
TGTGCCTTCTAGTTGCCAGCCATCTGTTGTTTGCCCCTCCCCCGTGCCTTCCTTGACC
CTGGAAGGTGCCACTCCCACTGTCCTTTCCTAATAAAATGAGGAAATTGCATCGCAT
TGTCTGAGTAGGTGTCATTCTATTCTGGGGGGTGGGGTGGGGCAGGACAGCAAGGG
GGAGGATTGGGAAGACAATAGCAGGCATGCTGGGGATGCGGTGGGCTCTATGGCTT
CTGAGGCGGAAAGAACCAGCTGGGGCTCGACTAGAGCTTGCGGAACCCTTCGAAGT
TCCTATTCTCTAGAAAGTATAGGAACTTCTGCACCATGGTATCACCATGCACTCACC
ATGGGAGATTGCGCCAACAGTATTACCACTGCCTAGCGCGTAGTTAGGTGTAATCGG
CTGAACTACCAGCGGCC (SEQ ID NO: 72)
10. Right Homology Arm: 1349 bp 3′ homology arm encompassing sequences immediately 3′
of the Tmprss2-crRNA_5 Cas9 cut site
GTATCAGTCTGAGCACATCTGTCCTCCGAGACCACCAGTGGCTCCCAATGGCTACAA
CTTGTATCCAGCCCAGTACTACCCATCTCCAGTGCCTCAGTATGCTCCGAGGATTAC
AACGCAAGCCTCAACATCTGTCATCCACACACATCCCAAGTCCTCAGGAGCACCGTG
CACCTCAAGTAAGACTCCTTAGCATTGTATCACAAGGGACTACAGTTTCTGGATCTG
CCTTCTCTTGTGGTCTCTTATTTCTCCTACCTTGTCAATAACACTGCACTGCCAACAG
GGCCAATCTTGTCTTACCTGCCTTCCACATTGGCTGGTCAGAGCCATGGGCTCTGCCT
CATCTCTCTTTGGCAGGGAGAGACCTTCCTCTCCCCTTGGCCTTTGGTGCAGCACCAC
TGTGTGCTTGCCCAGTTCAGCTGGACTGTTAGGGCATGGTTTCTCAGTCCCTTCTTGC
CTCTGTGCTTATGGATGTGTTGTCTTGAATCAACAGTAAGCACTCAAGGTTGGGAGT
GAACTGTCTTAGATGTTATTGATGTGCTCGGAACACGGAGGGCTCAGGGTTAAGATG
CCTCCTGTGACTAGCCAGAGATCTGTTGGAGCTCTCACCTCTGCCAGTTCTTGCCCTA
GAAGAAGAAGGGTCTTCCCCACAAGGGGGATTTCTCCCATTCCAGGGCCTGCTCAA
ATACTTTCTAGGGGAGCCAGGCTGCCTCTCCATGGCAAAATTACTCTCCCAGCCCTG
GATGTATCATGTCTTCCCCATAGCCCCTTACCCCCCCCCCCATAGCCCCAGAACAGT
GCTGAAGGTTGAATATGAAAGCCAAATCTCTTTGCCCTCTCCTGTGGCCCTTTCATTT
GATGATTACTCTCACCATTAGATTCCTTACCTCTATGTCCCTCTACCTTTTAATTCTGG
CTGTTCTCAGTGTTGCTTCTCTGGTGTCAAGGACACCAGGGACTCTATTGATAAGCT
ACTTTATATATTTATTTTTTAAAACTGAGCTTGTGTGTATTACATATGTGCCAGTGCC
TATGGAGGCCAGAAGGTGTTGGATCCTCTAGAGCTGGAGTCCAAGGTATTTTCGGTG
GTTGTAAACACTCAACACGAATGCTAGGAACCAAATTCGGATCCTCTGGAAGAGCA
GTAGATGTTCCTAACTGATGGCTATTTCTCCAGCTCCACACCAGCTTTCTAGCAGAAT
TGCTGCTTTATGAGATGAGCACCTGTCTGTGTGTTTCATGTGTCGTTCAAGGTTGATA
CAAAAGTGAATCCTGTGGGCCACCCATACTTCATCAGCAGTCATGTGATAGACCTTA
GGCCTCAGGAATCTGTTTGTCCCACTTGG (SEQ ID NO: 74)
Rat ACE2 (mRatBn7,2 (GCF_015227675,2)
acggggaaagatgtcaagctcctgctggctccttctcagccttgttgctgttgctactgctcagtccctcatcgaggaaa
aggccgagagctttttaaacaagtttaacc (SEQ ID NO: 75)
gRNA1 (no PAM)
GAAAGATGTCAAGCTCCTGC (SEQ ID NO: 76)
gRNA2 (no PAM)
TACTGCTCAGTCCCTCATCG (SEQ ID NO: 77)
Rat TMPRSS2 (mRatBn7,2 (GCF_015227675,2)
cacaggcaggatggcattgaactcagtaagtggttcatcttacgtctctggccctttccttgttccttcgcccctctggtat
cttccacaggtgtccatcagtaatttta (SEQ ID NO: 78)
gRNA1 (no PAM)
AAATTACTGATGGACACCTG (SEQ ID NO: 79)
gRNA2 (no PAM)
AGTGGTTCATCTTACGTCTC (SEQ ID NO: 80)
Hamster ACE2 (CriGri_1.0 (GCF_000223135,1)
Acggggaaagatgtcaagctcctcctggctccttctcagccttgttgctgtaactactgctcagtccatcatcgaggaa
caggccaagacatttttagac (SEQ ID NO: 81)
gRNA1 (no PAM)
AACAAGGCTGAGAAGGAGCC (SEQ ID NO: 82)
gRNA2 (no PAM)
GAAAGATGTCAAGCTCCTCC (SEQ ID NO: 83)
Hamster TMPRSS2 (CriGri_1.0 (GCF_000223135,1)
cacagccaggatggctttgaactcagtaagtggtcgattgttaaatctctggccttctccttgtccattcatccttctgttat
cttccatgggtgtccatctgtaatttt (SEQ ID NO: 84)
gRNA1 (no PAM)
ATCCTTCTGTTATCTTCCAT (SEQ ID NO: 85)
gRNA2 (no PAM)
TACTGAGTTCAAAGCCATCC (SEQ ID NO: 86)
Guinea Pig ACE2 (Cavpor3.0 (GCF_000151735.1)
tgttcagtggatgagatctggtggacggggaaagatgtccggctctttctggttcctgctcaatcttgttgctgtaactac
tgctcagttcaacctcgaggaacaggcca (SEQ ID NO: 87)
gRNA1 (no PAM)
TCAGTTCAACCTCGAGGAAC (SEQ ID NO: 88)
gRNA2 (no PAM)
TACTGCTCAGTTCAACCTCG (SEQ ID NO: 89)
Guinea Pig TMPRSS2 (Cavpor3.0 (GCF_000151735.1)
tgacctcagcatggatttgaactcagtaagtagttaatgattacctccctgccctccccccttccctgcattcccagcagt
gcaagggggcagctggaggaggggtaggt (SEQ ID NO: 90)
gRNA1 (noPAM)
CTGAGTTCAAATCCATGCTG (SEQ ID NO: 91)
gRNA2 (no PAM)
CCTTGCACTGCTGGGAATGC (SEQ ID NO: 92)

What is claimed is:

1. A construct comprising a human ACE2 cDNA minigene, the human ACE2 cDNA minigene comprising a start codon, and an SV40 mini-intron comprising a splice donor site and a splice acceptor site within the cDNA sequence of human ACE2.

2. The construct of claim 1, comprising a sequence having at least 80% sequence homology to SEQ ID NO: 1 [pSYNB026_C57BL6_Ace2_Humanizing Construct], or any fragment thereof.

3. The construct of claim 1, comprising a sequence having at least 80% sequence homology to SEQ ID NO: 2 [pSYNB028_Ace2_NEO_BalbC], or any fragment thereof.

4. The construct of claim 1, further comprising a mouse promotor sequence for the mouse ACE2 gene.

5. A genetically-modified mouse comprising in its genome the human ACE2 cDNA of minigene of claim 1 and a start codon of native mouse ACE2,
wherein the start codon of the human ACE2 cDNA minigene is upstream of the start codon of native mouse ACE2, and
wherein the human ACE2 cDNA minigene disrupts expression of native mouse ACE2.

6. The genetically-modified mouse of claim 5, wherein no more than 20 amino acids are encoded between the start codon of the human ACE2 cDNA minigene and the start codon of native mouse ACE2.

7. The genetically-modified mouse of claim 5, wherein the mouse is a C57BL/6 mouse.

8. The genetically-modified mouse of claim 5, wherein the mouse is a BALB/c mouse.

9. The genetically-modified mouse of claim 5, wherein the human cDNA minigene is under control of a promotor sequence for the native mouse ACE2 gene.

10. A construct comprising a human TMPRSS2 cDNA minigene, the human TMPRSS2 cDNA minigene comprising a start codon, and an SV40 mini-intron comprising a splice donor site and a splice acceptor site within the cDNA sequence of human TMPRSS2.

11. The construct of claim 8, comprising a sequence having at least 80% sequence homology to SEQ ID NO: 3 [pSYNB027_C57BL6_Tmprss2_Humanizing Construct], or any fragment thereof.

12. The construct of claim 8, comprising a sequence having at least 80% sequence homology to SEQ ID NO: 4 [pSYNB029_BalbC_Tmprss2_NEO Humanizing Construct], or any fragment thereof.

13. The construct of claim 10, further comprising a mouse promotor sequence for the mouse TMPRSS2 gene.

14. A genetically-modified mouse comprising in its genome the human TMPRSS2 cDNA minigene of claim 10 and a start codon for native mouse TMPRSS2,
wherein the start codon of the human TMPRSS2 cDNA minigene is upstream of the start codon for native mouse TMPRSS2, and
wherein the human TMPRSS2 cDNA minigene disrupts expression of native mouse TMPRSS2.

15. The genetically-modified mouse of claim 14, wherein no more than 20 amino acids are encoded between the start codon of the human TMPRSS2 cDNA minigene and the start codon of native mouse TMPRSS2.

16. The genetically-modified mouse of claim 14, wherein the mouse is a C57BL/6 mouse.

17. The genetically-modified mouse of claim 14, wherein the mouse is a BALB/c mouse.

18. The genetically-modified mouse of claim 14, further comprising in its genome a human ACE2 cDNA minigene, the human ACE2 cDNA minigene comprising a start codon, and an SV40 mini-intron comprising a splice donor site and a splice acceptor site within the cDNA sequence of the human ACE2 cDNA minigene.

19. The genetically-modified mouse of claim 15, wherein the start codon of the human ACE2 cDNA minigene is upstream of the start codon for native mouse ACE2, and wherein the human ACE2 cDNA minigene disrupts expression of native mouse ACE2

20. The genetically-modified mouse of claim 14, wherein the human TMPRSS2 cDNA minigene is under control of the promotor sequence for the native mouse TMPRSS2 gene.

21. A genetically-modified mouse comprising in its genome at least one chromosome comprising a nucleic acid sequence encoding a human cDNA ACE2 minigene, wherein the mouse does not express endogenous ACE2.

22. The genetically-modified mouse of claim 21, wherein the nucleic acid sequence encoding the human ACE2 is operably linked to an endogenous regulatory element at the endogenous ACE2 gene locus in the at least one chromosome.

23. A genetically-modified mouse comprising it its genome at least one chromosome comprising a nucleic acid sequence encoding a human cDNA TMPRSS2 minigene, wherein the mouse does not express endogenous TMPRSS2.

24. The genetically-modified mouse of claim 23, wherein the nucleic acid sequence encoding the human TMPRSS2 is operably linked to an endogenous regulatory element at the endogenous TMPRSS2 gene locus in the at least one chromosome.

25. A method of making a genetically-modified mouse, the method comprising replacing in at least one cell of the animal, at an endogenous ACE2 gene locus, a sequence encoding a region of an endogenous ACE2 with a sequence encoding a corresponding region of human cDNA ACE2 minigene.

26. The method of claim 25, wherein the mouse comprises a start codon of the human ACE2 cDNA minigene and a start codon of native mouse ACE2 gene, and wherein the start codon of the human ACE2 cDNA minigene is less than 20 amino acids from the start codon of native mouse ACE2 gene.

27. The method of claim 25, wherein the mouse expresses the human cDNA ACE2 minigene.


SEQUENCE LISTING (132 sequences)

SEQ ID NO: 1 - DNA, 6671 bp, Artificial Sequence
  pSYNB026_C57BL6_Ace2_Humanizing Construct
gagttctcagctgagttgtaagcaggtaagtgaaagggaaagaggcacctgataaagtcagctgtaggactagagtttagcatgtctcctgagaaatagaaaatgactgcttgaaactttaccaaagccacacaggaagcatcaaactccctatggagtggagaagagtcttataattttttaaatgggcagagaaatgaatttatttttaatttttagagacagggtttctttgtatagctctagctgtctttgattggtagacaaagctgtcctcaaactcagagatcttccttcctttgtctcctgagtgctgggattaaaggcatggaccaccactgccctgccccattctctccattaattttaagtgaatgcttgcaaaagctcacttctttggtgaacagcttcctttacaaataagtacctttgccttcgtttttataggattcttaaaaagaaaaaaaagattcagccaggtggttgtggtgcacacctttaatcccagcagtcaggaggcagaggaaagcagatctcttgagtttgaggctagcctagtctacagagggagttccaggacagccaaggctacagagaggaactgtctaaaaacaccaagaaagagagaaaggagagagggagaggatggatagcttattgatagaattgtcagaaaaggctataagttccaatatgtgtcccatgatttctaagtctagccctttctgttatagtaaaatcatagtacaccctcctcctccagtgtatctttaacagcttttaaggaacatattaactaaatgtccaggttttgatttggccataaaatgttagcaaagctaaggttttctaggattaatgaataacatgtctttatttagtttacttaaaaaaatcattctaaaatatctgtttacatatctgtcctctccaggattaacttcatattggtccagcagcttgtttactgttctcttctgtttcttcttctgctttttttttcttctcttctcagtgcccaacccaagttcaaaggctgatgagagagaaaaactcatgaagagattttactctagggaaagttgctcagtggatgggatcttggcgcacggggaaagatgtccagctcctcctcagtccaccattgaggaacaggccaagacatttttggacaagtttaaccacgaagccgaagacctgttctatcaaagttcacttgcttcttggaattataacaccaatattactgaagagaatgtccaaaacatggtaagtttagtctttttgtcttttatttcttgtcccggatccggtggtggtgcaaatcaaagaactgctcctcagtggattttgcctttacttctagaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactccttgtgcttacttatgtgctggggcttctttacgttttgtctgcttttcagttctatgaacaaagttatcatctgagtgtctggggctcctggccttccaaaaaaaagtgcctagcaaaaaagcaccgactcggtgccactttttcaagttgataacggactagccttattttaacttgctatttctagctctaaaaccctggctccttctcagccttcaaacaaggcttttctccaagggatatttatagtctcaaaacacacaattactttacagttagggtgagtttccttttgtgctgttttttaaaataataatttagtatttgtatctcttataaatatttttttccctctctagctttacaataattttcccagaacttgtttattgcagcttataatggttacaaataaagcaatagcatcacaaatttcacaaataaagcatttttttcactgcattctagttgtggtttgtccaaactcatcaatgtatcttatcatgtctggatgaagttcctattccgaagttcctattctctagaaagtataggaacttctgcaccatggtatcaccatgcactcaccatgggagattgcgccaacagtattaccactgcctagcgcgtagttaggtgtaatcggctgaactaccagcggccgccttctcagccttgttgctgttactactgctcagtccctcaccgaggaaaatgccaagacatttttaaacaactttaatcaggaagctgaagacctgtcttatcaaagttcacttgcttcttggaattataatactaacattactgaagaaaatgcccaaaagatggtaagttcttgaggctacccagggggttattgattgcttcttaaagatcagaattactgcctataaaactggataaggaaatcatagagatctctcaagtgtgaggatgagtgactgcctctgtagctctgatcctagtctcccagatggctaaattcaattgaccttagagttcatctggaaaattgttatgaatgaattatttgcccagattccaaagatgagtgaaaatgtttaataaagttgccatcactattctcattatatttggtatgtaaagcattcatggaaatgttctaagtcgttattgagccaataattttctttagcttataatgccaacaggtctatccgagaactacaaatgacatattaactgaaaaatgcaactggggtttactgaaggcagcagcttagtaattaaggtaaccatggcttaggtgaaactggacctgggaattccttctttcattgacacagagctctgaggaatttccaaaggtcacagaagaaaagctataattaaactagtcccaaaaaatctcagcctactctgggaaagcagcatattttgtttgacaagtgcaaggacttagaacttttttttttctcactgatcctgaagtgccttttaagtatagttaagtggtggaaaattgagcaactatttaagaaaagactcttttttttcttcttccagcaatgctttccttcaaaacggtagcttcaaaacttcctgtcttttaaatgatcagggggctgtgtgtttaaattattgccattcatagaacagagtgggtctgaggatgcctgtttcctttgaaattctatgccccctcccagttttctaaaatttaagaaaccacagagactttgacaatgtagttgccaaatgagttgcttttaactgctctaatagtttggtcttaccggtgttgtttttagtgtaactttttttcccctcagtttttattttatttctgttggagatcaccaacgtgtactgtagattttctgagctgaatggttcaaggtgagactaatattatacttgaaagcctagcctggagctgacatccagatcctggctgtaccactttttagctgggtaaacttgaattcattaaatggacagtctgggtcttagttactttttgttttaagatgtattttaattttaaaactatgtgtatgtgtgtgtgctcccgaatataagtatagatgcccatgaaggtcagagatattggaacccccaggagtggtgcagcaggctattgtgtgccaccagacctagatgctggcaacttgcaacctctgcaagggaagtacatatccttaaccatcgagccacctttctaccccagtctta

SEQ ID NO: 2 - DNA, 8500 bp, Artificial Sequence
  pSYNB028_Ace2_NEO_BalbC
gagttctcagctgagttgtaagcaggtaagtgaaagggaaagaggcacctgataaagtcagctgtaggactagagtttagcatgtctcctgagaaatagaaaatgactgcttgaaactttaccaaagccacacaggaagcatcaaactccctatggagtggagaagagtcttataattttttaaatgggcagagaaatgaatttatttttaatttttagagacagggtttctttgtatagctctagctgtctttgattggtagacaaagctgtcctcaaactcagagatcttccttcctttgtctcctgagtgctgggattaaaggcatggaccaccactgccctgccccattctctccattaattttaagtgaatgcttgcaaaagctcacttctttggtgaacagcttcctttacaaataagtacctttgccttcgtttttataggattcttaaaaagaaaaaaaagattcagccaggtggttgtggtgcacacctttaatcccagcagtcaggaggcagaggaaagcagatctcttgagtttgaggctagcctagtctacagagggagttccaggacagccaaggctacagagaggaactgtctaaaaacaccaagaaagagagaaaggagagagggagaggatggatagcttattgatagaattgtcagaaaaggctataagttccaatatgtgtcccatgatttctaagtctagccctttctgttatagtaaaatcatagtacaccctcctcctccagtgtatctttaacagcttttaaggaacatattaactaaatgtccaggttttgatttggccataaaatgttagcaaagctaaggttttctaggattaatgaataacatgtctttatttagtttacttaaaaaaatcattctaaaatatctgtttacatatctgtcctctccaggattaacttcatattggtccagcagcttgtttactgttctcttctgtttcttcttctgctttttttttcttctcttctcagtgcccaacccaagttcaaaggctgatgagagagaaaaactcatgaagagattttactctagggaaagttgctcagtggatgggatcttggcgcacggggaaagatgtccagctcctcctggcttctgctttctctggtggcagtgacaacagcacagtccaccattgaggaacaggccaagacatttttggacaagtttaaccacgaagccgaagacctgttctatcaaagttcacttgcttcttggaattataacaccaatattactgaagagaatgtccaaaacatggtaagtttagtctttttgtcttttatttcttgtcccggatccggtggtggtgcaaatcaaagaactgctcctcagtggattttgcctttacttctagaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactccttgtgcttacttatgtgctggggcttctttacgttttgtctgcttttcagttctatgaacaaagttatcatctgagtgtctggggctcctggccttccaaaaaaaagtgcctagcaaaaaagcaccgactcggtgccactttttcaagttgataacggactagccttattttaacttgctatttctagctctaaaaccctggctccttctcagccttcaaacaaggcttttctccaagggatatttatagtctcaaaacacacaattactttacagttagggtgagtttccttttgtgctgttttttaaaataataatttagtatttgtatctcttataaatatttttttccctctctagctttacaataattttcccagaacttgtttattgcagcttataatggttacaaataaagcaatagcatcacaaatttcacaaataaagcatttttttcactgcattctagttgtggtttgtccaaactcatcaatgtatcttatcatgtctggatgaagttcctattccgaagttcctattctctagaaagtataggaacttcaggtctgaagaggagtttacgtccagccaagctagcttggctgcaggtcgtcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttcgaagttcctattctctagaaagtataggaacttctgcaccatggtatcaccatgcactcaccatgggagattgcgccaacagtattaccactgcctagcgcgtagttaggtgtaatcggctgaactaccagcggccgccttctcagccttgttgctgttactactgctcagtccctcaccgaggaaaatgccaagacatttttaaacaactttaatcaggaagctgaagacctgtcttatcaaagttcacttgcttcttggaattataatactaacattactgaagaaaatgcccaaaagatggtaagttcttgaggctacccagggggttattgattgcttcttaaagatcagaattactgcctataaaactggataaggaaatcatagagatctctcaagtgtgaggatgagtgactgcctctgtagctctgatcctagtctcccagatggctaaattcaattgaccttagagttcatctggaaaattgttatgaatgaattatttgcccagattccaaagatgagtgaaaatgtttaataaagttgccatcactattctcattatatttggtatgtaaagcattcatggaaatgttctaagtcgttattgagccaataattttctttagcttataatgccaacaggtctatccgagaactacaaatgacatattaactgaaaaatgcaactggggtttactgaaggcagcagcttagtaattaaggtaaccatggcttaggtgaaactggacctgggaattccttctttcattgacacagagctctgaggaatttccaaaggtcacagaagaaaagctataattaaactagtcccaaaaaatctcagcctactctgggaaagcagcatattttgtttgacaagtgcaaggacttagaacttttttttttctcactgatcctgaagtgccttttaagtatagttaagtggtggaaaattgagcaactatttaagaaaagactcttttttttcttcttccagcaatgctttccttcaaaacggtagcttcaaaacttcctgtcttttaaatgatcagggggctgtgtgtttaaattattgccattcatagaacagagtgggtctgaggatgcctgtttcctttgaaattctatgccccctcccagttttctaaaatttaagaaaccacagagactttgacaatgtagttgccaaatgagttgcttttaactgctctaatagtttggtcttaccggtgttgtttttagtgtaactttttttcccctcagtttttattttatttctgttggagatcaccaacgtgtactgtagattttctgagctgaatggttcaaggtgagactaatattatacttgaaagcctagcctggagctgacatccagatcctggctgtaccactttttagctgggtaaacttgaattcattaaatggacagtctgggtcttagttactttttgttttaagatgtattttaattttaaaactatgtgtatgtgtgtgtgctcccgaatataagtatagatgcccatgaaggtcagagatattggaacccccaggagtggtgcagcaggctattgtgtgccaccagacctagatgctggcaacttgcaacctctgcaagggaagtacatatccttaaccatcgagccacctttctaccccagtctta

SEQ ID NO: 3 - DNA, 6994 bp, Artificial Sequence
  pSYNB027_C57BL6_Tmprss2_Humanizing Construct
gtcatccaggacaaagtgctaggtgtatcttgttctttcctgctggagaggggaacacattcttagacacctggtttctcaatctggaatgctctgttaatatgggaatagctttcgctgtcccaccctgaggtttcaagttataatctttggacctttgaaggagagtgctagtgagttattaagcacagatgattaaccttggagtgttggtattgccttaaaattctcaacctttaggccagcaagacagctccataaggtaagagatgacctgcattcaatctttgtggccacagggtggaaggagacaactggctctcacaggttgtcctatgatctctggttacacacacgtgcaacacacacaataaatacatgagcttttaaaagtctgagcatccagtggtccctgctgaggatatctgcttccagaagatgatttttctgtcattcctgaggcccatgagagaacctgagcagaaagagagctggagaggtttggggtaagaaccattagacatgcatagggagacctctctgggagaggtccagctactgagagtagggagagtggaaataacaatgccaacctgtgaacttgctactgaacctgacaactgctgggccatactaggagtacagtctggggaaggtcacgtgaatggactgagtgaaaggattgatacccaagaagtggatcctgccctttttacattcacacagcacttactttgacctccaagttcccctgaaagggcacctacaccctcagcaccaaaatgcatacactctatagggatgttcacactgagctaacaccatcctggtgagaacagatgggtaaaggactaccaactacagctgcatgagagaaggaccctggaagagaatggccacagagtctgcattgtgggtcccttctagcagtttctcagctcagctgaaaagtgtatttactacacccaaggttgcacacattatctgaatatgaggtccctttagcaggtgagttctcagatatgagctatatcctgaaccttgacattaacctggctcaaaatttcagacctcaaggctcagggggtgatttagaggaggaaccagagaatgcgactctaactgtgtgacttttgaccccaaacttgtatgtatgttttctacagaatcttagttatcggtcaccttttctttatttcaaagaacaaataatgaaaaaaaaaaaatggtccaggaaaccaaagctaatgaaacacagatagttgagcgttctagttagtgcagaaacaagagtctagtcatgttaagcaagttgctgtgatgacagagtgattggcaatctgctggctctctccctgttttctgtgaacaatgctaactttccaaacctttagagtgagtgagagctgtaactgatttatctcctttcccattgtttaggggtcacctccaggaatcggaccttgctatgagaaccacggaggtggaagcggagctactaacttcagcctgctgaagcaggctggagacgtggaggagaaccctggacccatggctttgaactcagtaagtttagtctttttgtcttttatttcttgtcccggatccggtggtggtgcaaatcaaagaactgctcctcagtggattttgcctttacttctaggggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggaccaaacttcatccttcaggtgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtatagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggcagacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggtggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaaatcaaggatgctcagtttaaggtacactgtttccatgttatgtttctacacattgctacctcagtgctcctggaaacttagcttttgatgtctccaagtagtccaccttcatttaactctttgaaactgtatcatctttgccaagtaagagtggtggcctatttcagctgctttgacaaaatgactggctcctgacttaacgttctataaatgaatgtgctgaagcaaagtgcccatggtggcggcgaagaagagaaagatgtgttttgttttggactctctgtggtcccttccaatgctgtgggtttccaaccaggggaagggtcccttttgcattgccaagtgccataaccatgagcactactctaccatggttctgcctcctggccaagcaggctggtttgcaagaatgaaatgaatgattctacagctaggacttaaccttgaaatggaaagtcatgcaatcccatttgcaggatctgtctgtgcacatgcctctgtagagagcagcattcccagggaccttggaaacagttggcactgtaaggtgcttgctccccaagacacatcctaaaaggtgttgtaatggtgaaaacgtcttccttctttattgccccttcttatttatgtgaacaactgtttgtctttttttgtatcttttttaaactgtaaagttcaattgtgaaaatgaatatcatgcaaataaattatgcaatttttttttcaaagtaactactgcatctttgaagttctgcctggtgagtaggaccagcctccatttccttataagggggtgatgttgaggctgctggtcagaggaccaaaggtgaggcaaggccagacttggtgctcctgtggttggtgccctcagttcctgcagcctgtcctgttggagaggtccctcaaatgactccttcttattattctattagtctgtttccatgctcctaataaagacatacccaagactgcaatttacaaaagaaagaagtttattggatttacaattccacatggctggggaggcctcacaatcatggcagaaagcaaggaagagcaaatcacatcttacatggatggcagcaggcagaaaaaaagtgcctagcaaaaaagcaccgactcggtgccactttttcaagttgataacggactagccttattttaacttgctatttctagctctaaaaccgggtatcagtctgagcacacaaacaaggcttttctccaagggatatttatagtctcaaaacacacaattactttacagttagggtgagtttccttttgtgctgttttttaaaataataatttagtatttgtatctcttataaatatttttttccctctctagctttacaataattttcccagaacttgtttattgcagcttataatggttacaaataaagcaatagcatcacaaatttcacaaataaagcatttttttcactgcattctagttgtggtttgtccaaactcatcaatgtatcttatcatgtctggatgaagttcctattccgaagttcctattctctagaaagtataggaacttctgcaccatggtatcaccatgcactcaccatgggagattgcgccaacagtattaccactgcctagcgcgtagttaggtgtaatcggctgaactaccagcggccgtatcagtctgagcacatctgtcctccgagaccaccagtggctcccaatggctacaacttgtatccagcccagtactacccatctccagtgcctcagtatgctccgaggattacaacgcaagcctcaacatctgtcatccacacacatcccaagtcctcaggagcactgtgcacctcaagtaagactccttagcattgtatcacaagggactacagcttctggatctgccttctcttgtggtctcttatttctcctaccttgtcaataacactgcactgccaacagggccaatcttgtcttacctgccttccacattggctggtcagagccatgggctctgcctcatctctctttggcagggagagaccttcctctccccttggcctttggtgcagcaccactgtgtgcttgcccagttcagctggactgttagggcatggtttctcagtcccttcttgcctctgtgcttatggatgtgttgtcttgaatcaacagtaagcactcaaggttgggagtgaactgtcttagatgttattgatgtgctcagaacacagagggctcagggttaagatacctcctgtgactagccagagatctgttggagctctcacctctgccagttcttgccctagaagaagaagggtcttccccacaagggggatttctcccattccagggcctgcccatatactttctaggggagccaggctgcctctccacggcaaaattactctcccagccctggatgtatcatgtcttccccatagccccttaccccccccccccccatagccccagaacagtgctgaaggttgaatatgaaagccgaatctctttgccctctcctgtggccctttcatttgatgattattctcaccattagattccttacctcttatgtccctctaccttttaattctggctgttctcagtgttgcctctctggtgtcaaggacaccagggactctattgataagctactttatatatttattttttaaaactgagcttgtttgtattacatatgtgccagtgcctatggaggccagaaggtgttggatcctctagagctggagtccaaggtattttcggtggttgtaaacactcaacatgaatgctaggaaccaaattcggatcctctggaagagcagtagatgttcctaactgatggctatttctccagctccacaccagctttctagcagagttgctgctttatgagatgagcacctgtctgtgtgtttcatgtatcgttcaaggttgatacaaaagtgaatcctgtgggccacccatacttcatcagcagtcatgtgatagaccttaggcctcaggaatctgtttgtcccacttgg

SEQ ID NO: 4 - DNA, 8806 bp, Artificial Sequence
  pSYNB029_BalbC_Tmprss2_NEO Humanizing Construct
gtcatccaggacaaagtgctaggtgtatcttgttctttcctgctggagaggggaacacatccttagacggtttggttctgggacagacactcacctggtttctcaatctggaatgctctgttaatatgggaatagctttcgctgtcccaccctgaggtttcaagttataatctttggacctttgaaggagagtgctagtgagttattaagcacagatgattaaccttggagtgttggtattgccttaaaattctcaacctttaggccagcaagacagctccataaggtaagagatgacctgcattcaatctttgtggccacagggtggaaggagacaactggctctcacaggttgtcctatgatctctggttacacacacgtgcaacacacacaataaatacatgagcttttaaaagtctgagcatccagtggtccctgctgaggatatctgcttccagaagatgatttttctgtcattcctgaggcccatgagagaacctgagcagaaagagagctggagaggtttggggtaagaaccattagacatgcatagggagacctctctgggagaggtccagctactgagagtagggagagtggaaataacaatgccaacctgtgaacttgctactgaacctgacaactgctgggccatactaggagtacagtctggggaaggtcacgtgaatggactgagtgaaaggattgatacccaagaagtggatcctgccctttttacattcacacagcacttactttgacctccaagttcccctgaaagggcacctacaccctcagcaccaaaatgcatacactctatagggatgttcacactgagctaacaccatcctggtgagaacagatgggtaaaggactaccaactacagctgcatgagagaaggaccctggaagagaatggccacaggagtctgcattgtgggtcccttctagcagtttctcagctcagctgaaaagtgtatttactacacccaaggttgcacacattatctgaatatgaggtccctttagcaggtgagttctcagatatgagctatatcctgaaccttgacattaacctggctcaaaatttcagacctcaaggctcagggggtgatttagaggaggaaccagagaatgcgactctaactgtgtgacttttgactccaaacttgtatgtatgttttctacagaatcttagttatcggtcaccttttctttatttcaaagaacaaataatgaaaaaaaaatggtccaggaaaccaaagctaatgaaacacagatagttgagcgttctagttagtgcagaaacaagagtctagtcatgttaagcaagttgctgtgatgacagagtgattggcaatctgctggctctctccctgttttctgtgaacaatgctaactttccaaacctttagagtgagtgagagctgtaactgatttatctcctttcccattgtttaggggtcacctccaggaatcggaccttgctatgagaaccacggaggtggaagcggagctactaacttcagcctgctgaagcaggctggagacgtggaggagaaccctggacccatggctttgaactcagtaagtttagtctttttgtcttttatttcttgtcccggatccggtggtggtgcaaatcaaagaactgctcctcagtggattttgcctttacttctaggggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggaccaaacttcatccttcaggtgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtatagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggcagacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggtggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaaatcaaggatgctcagtttaaggtacactgtttccatgttatgtttctacacattgctacctcagtgctcctggaaacttagcttttgatgtctccaagtagtccaccttcatttaactctttgaaactgtatcatctttgccaagtaagagtggtggcctatttcagctgctttgacaaaatgactggctcctgacttaacgttctataaatgaatgtgctgaagcaaagtgcccatggtggcggcgaagaagagaaagatgtgttttgttttggactctctgtggtcccttccaatgctgtgggtttccaaccaggggaagggtcccttttgcattgccaagtgccataaccatgagcactactctaccatggttctgcctcctggccaagcaggctggtttgcaagaatgaaatgaatgattctacagctaggacttaaccttgaaatggaaagtcatgcaatcccatttgcaggatctgtctgtgcacatgcctctgtagagagcagcattcccagggaccttggaaacagttggcactgtaaggtgcttgctccccaagacacatcctaaaaggtgttgtaatggtgaaaacgtcttccttctttattgccccttcttatttatgtgaacaactgtttgtctttttttgtatcttttttaaactgtaaagttcaattgtgaaaatgaatatcatgcaaataaattatgcaatttttttttcaaagtaactactgcatctttgaagttctgcctggtgagtaggaccagcctccatttccttataagggggtgatgttgaggctgctggtcagaggaccaaaggtgaggcaaggccagacttggtgctcctgtggttggtgccctcagttcctgcagcctgtcctgttggagaggtccctcaaatgactccttcttattattctattagtctgtttccatgctcctaataaagacatacccaagactgcaatttacaaaagaaagaagtttattggatttacaattccacatggctggggaggcctcacaatcatggcagaaagcaaggaagagcaaatcacatcttacatggatggcagcaggcagaaaaaaagtgcctagcaaaaaagcaccgactcggtgccactttttcaagttgataacggactagccttattttaacttgctatttctagctctaaaaccgggtatcagtctgagcacacaaacaaggcttttctccaagggatatttatagtctcaaaacacacaattactttacagttagggtgagtttccttttgtgctgttttttaaaataataatttagtatttgtatctcttataaatatttttttccctctctagctttacaataattttcccagaacttgtttattgcagcttataatggttacaaataaagcaatagcatcacaaatttcacaaataaagcatttttttcactgcattctagttgtggtttgtccaaactcatcaatgtatcttatcatgtctggatgaagttcctattccgaagttcctattctctagaaagtataggaacttcaggtctgaagaggagtttacgtccagccaagctagcttggctgcaggtcgtcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttcgaagttcctattctctagaaagtataggaacttctgcaccatggtatcaccatgcactcaccatgggagattgcgccaacagtattaccactgcctagcgcgtagttaggtgtaatcggctgaactaccagcggccgtatcagtctgagcacatctgtcctccgagaccaccagtggctcccaatggctacaacttgtatccagcccagtactacccatctccagtgcctcagtatgctccgaggattacaacgcaagcctcaacatctgtcatccacacacatcccaagtcctcaggagcaccgtgcacctcaagtaagactccttagcattgtatcacaagggactacagtttctggatctgccttctcttgtggtctcttatttctcctaccttgtcaataacactgcactgccaacagggccaatcttgtcttacctgccttccacattggctggtcagagccatgggctctgcctcatctctctttggcagggagagaccttcctctccccttggcctttggtgcagcaccactgtgtgcttgcccagttcagctggactgttagggcatggtttctcagtcccttcttgcctctgtgcttatggatgtgttgtcttgaatcaacagtaagcactcaaggttgggagtgaactgtcttagatgttattgatgtgctcggaacacggagggctcagggttaagatgcctcctgtgactagccagagatctgttggagctctcacctctgccagttcttgccctagaagaagaagggtcttccccacaagggggatttctcccattccagggcctgctcaaatactttctaggggagccaggctgcctctccatggcaaaattactctcccagccctggatgtatcatgtcttccccatagccccttacccccccccccatagccccagaacagtgctgaaggttgaatatgaaagccaaatctctttgccctctcctgtggccctttcatttgatgattactctcaccattagattccttacctctatgtccctctaccttttaattctggctgttctcagtgttgcttctctggtgtcaaggacaccagggactctattgataagctactttatatatttattttttaaaactgagcttgtgtgtattacatatgtgccagtgcctatggaggccagaaggtgttggatcctctagagctggagtccaaggtattttcggtggttgtaaacactcaacacgaatgctaggaaccaaattcggatcctctggaagagcagtagatgttcctaactgatggctatttctccagctccacaccagctttctagcagaattgctgctttatgagatgagcacctgtctgtgtgtttcatgtgtcgttcaaggttgatacaaaagtgaatcctgtgggccacccatacttcatcagcagtcatgtgatagaccttaggcctcaggaatctgtttgtcccacttgg

SEQ ID NO: 5 - DNA, 20 bp, Artificial Sequence
  protospacer sequence
aaggctgagaaggagccagg

SEQ ID NO: 6 - protein, 12 aa, Artificial Sequence
  signal peptide coding sequence
WLLLSLVAVTTA

SEQ ID NO: 7 - DNA, 25 bp, Artificial Sequence
  Fwd Primer SB550
ggtctcacaagagacagctatagag

SEQ ID NO: 8 - DNA, 22 bp, Artificial Sequence
  Rev Primer SB196
tgaggagcagttctttgatttg

SEQ ID NO: 9 - DNA, 23 bp, Artificial Sequence
  Fwd Primer SB466
tgcaccatggtatcaccatgcac

SEQ ID NO: 10 - DNA, 23 bp, Artificial Sequence
  Rev Primer SB551
cagctgatcattcatactctccc

SEQ ID NO: 11 - DNA, 20 bp, Artificial Sequence
  Fwd Primer SB175
caagatggattgcacgcagg

SEQ ID NO: 12 - DNA, 23 bp, Artificial Sequence
  Rev Primer SB177
aaaccacaactagaatgcagtga

SEQ ID NO: 13 - DNA, 23 bp, Artificial Sequence
  Fwd Primer SB319
ccgtaaagcactaaatcggaacc

SEQ ID NO: 14 - DNA, 24 bp, Artificial Sequence
  Rev Primer SB553
aagatctctgagtttgaggacagc

SEQ ID NO: 15 - DNA, 21 bp, Artificial Sequence
  Fwd Primer SB552
agctgaatggttcaaggtgag

SEQ ID NO: 16 - DNA, 23 bp, Artificial Sequence
  Rev Primer SB315
cttttgctcacatgttctttcct

SEQ ID NO: 17 - DNA, 20 bp, Artificial Sequence
  protospacer sequence
tgtgctcagactgatacccg

SEQ ID NO: 18 - DNA, 24 bp, Artificial Sequence
  Fwd Primer SB546
gaggtccactccctagtgattaag

SEQ ID NO: 19 - DNA, 20 bp, Artificial Sequence
  Rev Primer SB269
gtccagggttctcctccacg

SEQ ID NO: 20 - DNA, 23 bp, Artificial Sequence
  Fwd Primer SB466
tgcaccatggtatcaccatgcac

SEQ ID NO: 21 - DNA, 22 bp, Artificial Sequence
  Rev Primer SB547
gggggtcattctgtacttctgg

SEQ ID NO: 22 - DNA, 23 bp, Artificial Sequence
  Fwd Primer SB177
aaaccacaactagaatgcagtga

SEQ ID NO: 23 - DNA, 23 bp, Artificial Sequence
  Rev Primer SB178
aaaagcaccgactcggtgccact

SEQ ID NO: 24 - DNA, 23 bp, Artificial Sequence
  Fwd Primer SB315
cttttgctcacatgttctttcct

SEQ ID NO: 25 - DNA, 21 bp, Artificial Sequence
  Rev Primer SB548
gagctgtcttgctggcctaaa

SEQ ID NO: 26 - DNA, 23 bp, Artificial Sequence
  Fwd Primer SB319
ccgtaaagcactaaatcggaacc

SEQ ID NO: 27 - DNA, 25 bp, Artificial Sequence
  Rev Primer SB549
cctaactgatggctatttctccagc

SEQ ID NO: 28 - DNA, 20 bp, Artificial Sequence
  protospacer sequence
aaggctgagaaggagccagg

SEQ ID NO: 29 - DNA, 24 bp, Artificial Sequence
  Fwd Primer Ace2-5LR-F1
gatgcgctttggatttcataatgc

SEQ ID NO: 30 - DNA, 26 bp, Artificial Sequence
  Rev Primer SV40Int-R5
cactgaggagcagttctttgatttgc

SEQ ID NO: 31 - DNA, 28 bp, Artificial Sequence
  Fwd Primer YCDS-F2
agttaggtgtaatcggctgaactaccag

SEQ ID NO: 32 - DNA, 27 bp, Artificial Sequence
  Rev Primer Ace2-3LR-R1
accctacaagcatcacacattcttctg

SEQ ID NO: 33 - DNA, 28 bp, Artificial Sequence
  Fwd Primer AMC-097
taatacgactcactatagggcgaattgg

SEQ ID NO: 34 - DNA, 29 bp, Artificial Sequence
  Rev Primer Ace2-5HR-R1
aggcaaaggtacttatttgtaaaggaagc

SEQ ID NO: 35 - DNA, 27 bp, Artificial Sequence
  Fwd Primer Ace2-3HR-F1
ttgacaatgtagttgccaaatgagttg

SEQ ID NO: 36 - DNA, 25 bp, Artificial Sequence
  Rev Primer AMC-107
ttgtgtggaattgtgagcggataac

SEQ ID NO: 37 - DNA, 28 bp, Artificial Sequence
  Fwd Primer Ace2-5PF
taaagatgatatgttccttgaggccagt

SEQ ID NO: 38 - DNA, 23 bp, Artificial Sequence
  Rev Primer Ace2-5PR
acacaatggggcacagcatgtaa

SEQ ID NO: 39 - DNA, 31 bp, Artificial Sequence
  Fwd Primer Ace2-3PF
gtaattggatactgtacctggcattagtttg

SEQ ID NO: 40 - DNA, 29 bp, Artificial Sequence
  Rev Primer Ace2-3PR
gcaaactcacagtctaattctgcaactca

SEQ ID NO: 41 - DNA, 27 bp, Artificial Sequence
  Fwd Primer Tmprss2-5LR-F1
gccagtgttccattttatgagtctctg

SEQ ID NO: 42 - DNA, 26 bp, Artificial Sequence
  Rev Primer SV40Int-R5
cactgaggagcagttctttgatttgc

SEQ ID NO: 43 - DNA, 28 bp, Artificial Sequence
  Fwd Primer YCDS-F2
agttaggtgtaatcggctgaactaccag

SEQ ID NO: 44 - DNA, 27 bp, Artificial Sequence
  Rev Primer Tmprss2-3LR-R1
ggtggatcagagcaaagaacagaagag

SEQ ID NO: 45 - DNA, 25 bp, Artificial Sequence
  Fwd Primer AMC-107
ttgtgtggaattgtgagcggataac

SEQ ID NO: 46 - DNA, 25 bp, Artificial Sequence
  Rev Primer Tmprss2-5HR-R1
tgtcttgctggcctaaaggttgaga

SEQ ID NO: 47 - DNA, 27 bp, Artificial Sequence
  Fwd Primer Tmprss2-3HR-F1
aggacaccagggactctattgataagc

SEQ ID NO: 48 - DNA, 28 bp, Artificial Sequence
  Rev Primer AMC-097
taatacgactcactatagggcgaattgg

SEQ ID NO: 49 - DNA, 23 bp, Artificial Sequence
  Fwd Primer Tmprss2-5PF
ctgcctgtccccatttcttcaag

SEQ ID NO: 50 - DNA, 28 bp, Artificial Sequence
  Rev Primer Tmprss2-5PR
gcacctgctcctacacacagttcttaaa

SEQ ID NO: 51 - DNA, 26 bp, Artificial Sequence
  Fwd Primer Tmprss2-3PF
tgctgtccctctctacttctgagtgg

SEQ ID NO: 52 - DNA, 26 bp, Artificial Sequence
  Rev Primer Tmprss2-3PR
gaagcccaagactgtagtctgaatgg

SEQ ID NO: 53 - DNA, 1134 bp, Artificial Sequence
  Left Homology Arm
gagttctcagctgagttgtaagcaggtaagtgaaagggaaagaggcacctgataaagtcagctgtaggactagagtttagcatgtctcctgagaaatagaaaatgactgcttgaaactttaccaaagccacacaggaagcatcaaactccctatggagtggagaagagtcttataattttttaaatgggcagagaaatgaatttatttttaatttttagagacagggtttctttgtatagctctagctgtctttgattggtagacaaagctgtcctcaaactcagagatcttccttcctttgtctcctgagtgctgggattaaaggcatggaccaccactgccctgccccattctctccattaattttaagtgaatgcttgcaaaagctcacttctttggtgaacagcttcctttacaaataagtacctttgccttcgtttttataggattcttaaaaagaaaaaaaagattcagccaggtggttgtggtgcacacctttaatcccagcagtcaggaggcagaggaaagcagatctcttgagtttgaggctagcctagtctacagagggagttccaggacagccaaggctacagagaggaactgtctaaaaacaccaagaaagagagaaaggagagagggagaggatggatagcttattgatagaattgtcagaaaaggctataagttccaatatgtgtcccatgatttctaagtctagccctttctgttatagtaaaatcatagtacaccctcctcctccagtgtatctttaacagcttttaaggaacatattaactaaatgtccaggttttgatttggccataaaatgttagcaaagctaaggttttctaggattaatgaataacatgtctttatttagtttacttaaaaaaatcattctaaaatatctgtttacatatctgtcctctccaggattaacttcatattggtccagcagcttgtttactgttctcttctgtttcttcttctgctttttttttcttctcttctcagtgcccaacccaagttcaaaggctgatgagagagaaaaactcatgaagagattttactctagggaaagttgctcagtggatgggatcttggcgcacggggaaagatgtccagctcctcct

SEQ ID NO: 54 - protein, 12 aa, Artificial Sequence
  signal peptide coding sequence
WLLLSLVAVTTA

SEQ ID NO: 55 - DNA, 51 bp, Artificial Sequence
  translation of signal peptide coding sequence with first 5 amino acids of mouse Ace2
atgtccagctcctcctggcttctgctttctctggtggcagtgacaacagca

SEQ ID NO: 56 - DNA, 2464 bp, Artificial Sequence
  hACE2 CDS human ACE2 cDNA coding sequence with SV40 intron inserted for enhanced expression
cagtccaccattgaggaacaggccaagacatttttggacaagtttaaccacgaagccgaagacctgttctatcaaagttcacttgcttcttggaattataacaccaatattactgaagagaatgtccaaaacatggtaagtttagtctttttgtcttttatttcttgtcccggatccggtggtggtgcaaatcaaagaactgctcctcagtggattttgcctttacttctagaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 57 - DNA, 872 bp, Artificial Sequence
  hACE2 3 primeUTR 872 bp human ACE2 3 prime UTR
aaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactc

SEQ ID NO: 58 - DNA, 100 bp, Artificial Sequence
  hACE2 3 prime 100 bp human intergenic sequence immediately 3prime of ACE2 polyadenylation region
cttgtgcttacttatgtgctggggcttctttacgttttgtctgcttttcagttctatgaacaaagttatcatctgagtgtctggggctcctggccttcca

SEQ ID NO: 59 - DNA, 240 bp, Artificial Sequence
  Ace2-gRNA32 Cassette Active genetics cassette comprised of a mouse U6 promoter-driven Ace2-gRNA32 guide RNA with 2xPolIII terminator, all in reverse orientation relative to Ace2
aaaaaaagtgcctagcaaaaaagcaccgactcggtgccactttttcaagttgataacggactagccttattttaacttgctatttctagctctaaaaccctggctccttctcagccttcaaacaaggcttttctccaagggatatttatagtctcaaaacacacaattactttacagttagggtgagtttccttttgtgctgttttttaaaataataatttagtatttgtatctcttata

SEQ ID NO: 60 - DNA, 326 bp, Artificial Sequence
  Splice-Trap FRT Cassette
aatatttttttccctctctagctttacaataattttcccagaacttgtttattgcagcttataatggttacaaataaagcaatagcatcacaaatttcacaaataaagcatttttttcactgcattctagttgtggtttgtccaaactcatcaatgtatcttatcatgtctggatgaagttcctattccgaagttcctattctctagaaagtataggaacttctgcaccatggtatcaccatgcactcaccatgggagattgcgccaacagtattaccactgcctagcgcgtagttaggtgtaatcggctgaactaccagcggccg

SEQ ID NO: 61 - DNA, 1535 bp, Artificial Sequence
  Right Homology Arm
ccttctcagccttgttgctgttactactgctcagtccctcaccgaggaaaatgccaagacatttttaaacaactttaatcaggaagctgaagacctgtcttatcaaagttcacttgcttcttggaattataatactaacattactgaagaaaatgcccaaaagatggtaagttcttgaggctacccagggggttattgattgcttcttaaagatcagaattactgcctataaaactggataaggaaatcatagagatctctcaagtgtgaggatgagtgactgcctctgtagctctgatcctagtctcccagatggctaaattcaattgaccttagagttcatctggaaaattgttatgaatgaattatttgcccagattccaaagatgagtgaaaatgtttaataaagttgccatcactattctcattatatttggtatgtaaagcattcatggaaatgttctaagtcgttattgagccaataattttctttagcttataatgccaacaggtctatccgagaactacaaatgacatattaactgaaaaatgcaactggggtttactgaaggcagcagcttagtaattaaggtaaccatggcttaggtgaaactggacctgggaattccttctttcattgacacagagctctgaggaatttccaaaggtcacagaagaaaagctataattaaactagtcccaaaaaatctcagcctactctgggaaagcagcatattttgtttgacaagtgcaaggacttagaacttttttttttctcactgatcctgaagtgccttttaagtatagttaagtggtggaaaattgagcaactatttaagaaaagactcttttttttcttcttccagcaatgctttccttcaaaacggtagcttcaaaacttcctgtcttttaaatgatcagggggctgtgtgtttaaattattgccattcatagaacagagtgggtctgaggatgcctgtttcctttgaaattctatgccccctcccagttttctaaaatttaagaaaccacagagactttgacaatgtagttgccaaatgagttgcttttaactgctctaatagtttggtcttaccggtgttgtttttagtgtaactttttttcccctcagtttttattttatttctgttggagatcaccaacgtgtactgtagattttctgagctgaatggttcaaggtgagactaatattatacttgaaagcctagcctggagctgacatccagatcctggctgtaccactttttagctgggtaaacttgaattcattaaatggacagtctgggtcttagttactttttgttttaagatgtattttaattttaaaactatgtgtatgtgtgtgtgctcccgaatataagtatagatgcccatgaaggtcagagatattggaacccccaggagtggtgcagcaggctattgtgtgccaccagacctagatgctggcaacttgcaacctctgcaagggaagtacatatccttaaccatcgagccacctttctaccccagtctta

SEQ ID NO: 62 - DNA, 1481 bp, Artificial Sequence
  Left Homology Arm
gtcatccaggacaaagtgctaggtgtatcttgttctttcctgctggagaggggaacacattcttagacacctggtttctcaatctggaatgctctgttaatatgggaatagctttcgctgtcccaccctgaggtttcaagttataatctttggacctttgaaggagagtgctagtgagttattaagcacagatgattaaccttggagtgttggtattgccttaaaattctcaacctttaggccagcaagacagctccataaggtaagagatgacctgcattcaatctttgtggccacagggtggaaggagacaactggctctcacaggttgtcctatgatctctggttacacacacgtgcaacacacacaataaatacatgagcttttaaaagtctgagcatccagtggtccctgctgaggatatctgcttccagaagatgatttttctgtcattcctgaggcccatgagagaacctgagcagaaagagagctggagaggtttggggtaagaaccattagacatgcatagggagacctctctgggagaggtccagctactgagagtagggagagtggaaataacaatgccaacctgtgaacttgctactgaacctgacaactgctgggccatactaggagtacagtctggggaaggtcacgtgaatggactgagtgaaaggattgatacccaagaagtggatcctgccctttttacattcacacagcacttactttgacctccaagttcccctgaaagggcacctacaccctcagcaccaaaatgcatacactctatagggatgttcacactgagctaacaccatcctggtgagaacagatgggtaaaggactaccaactacagctgcatgagagaaggaccctggaagagaatggccacagagtctgcattgtgggtcccttctagcagtttctcagctcagctgaaaagtgtatttactacacccaaggttgcacacattatctgaatatgaggtccctttagcaggtgagttctcagatatgagctatatcctgaaccttgacattaacctggctcaaaatttcagacctcaaggctcagggggtgatttagaggaggaaccagagaatgcgactctaactgtgtgacttttgaccccaaacttgtatgtatgttttctacagaatcttagttatcggtcaccttttctttatttcaaagaacaaataatgaaaaaaaaaaaatggtccaggaaaccaaagctaatgaaacacagatagttgagcgttctagttagtgcagaaacaagagtctagtcatgttaagcaagttgctgtgatgacagagtgattggcaatctgctggctctctccctgttttctgtgaacaatgctaactttccaaacctttagagtgagtgagagctgtaactgatttatctcctttcccattgtttaggggtcacctccaggaatcggaccttgctatgagaaccacgg

SEQ ID NO: 63 - DNA, 13 bp, Artificial Sequence
  GGSG linker
aggtggaagcgga

SEQ ID NO: 64 - DNA, 57 bp, Artificial Sequence
  P2A porcine teschovirus 2A self-cleaving peptide sequence
gctactaacttcagcctgctgaagcaggctggagacgtggaggagaaccctggaccc

SEQ ID NO: 65 - DNA, 1576 bp, Artificial Sequence
  hTMPRSS2 CDS human TMPRSS2 cDNA coding sequence with SV40 intron inserted for enhanced expression
atggctttgaactcagtaagtttagtctttttgtcttttatttcttgtcccggatccggtggtggtgcaaatcaaagaactgctcctcagtggattttgcctttacttctaggggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggaccaaacttcatccttcaggtgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtatagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggcagacggctaa

SEQ ID NO: 66 - DNA, 1837 bp, Artificial Sequence
  hTMPRSS2 3 prime UTR 1837 bp human TMPRSS2 3 prime UTR
tccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggtggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaaatcaaggatgctcagtttaaggtacactgtttccatgttatgtttctacacattgctacctcagtgctcctggaaacttagcttttgatgtctccaagtagtccaccttcatttaactctttgaaactgtatcatctttgccaagtaagagtggtggcctatttcagctgctttgacaaaatgactggctcctgacttaacgttctataaatgaatgtgctgaagcaaagtgcccatggtggcggcgaagaagagaaagatgtgttttgttttggactctctgtggtcccttccaatgctgtgggtttccaaccaggggaagggtcccttttgcattgccaagtgccataaccatgagcactactctaccatggttctgcctcctggccaagcaggctggtttgcaagaatgaaatgaatgattctacagctaggacttaaccttgaaatggaaagtcatgcaatcccatttgcaggatctgtctgtgcacatgcctctgtagagagcagcattcccagggaccttggaaacagttggcactgtaaggtgcttgctccccaagacacatcctaaaaggtgttgtaatggtgaaaacgtcttccttctttattgccccttcttatttatgtgaacaactgtttgtctttttttgtatcttttttaaactgtaaagttcaattgtgaaaatgaatatcatgcaaataaattatgcaatttttttttcaaagtaactactgcatctttgaagttctgcctggtgagtaggaccagcctccatttccttataagggggtgatgttgaggctgctggtcagaggaccaaaggtgaggcaaggccagacttggtgctcctgtggttggtgccctcagttcctgcagcctgtcctgttggagaggtccctcaaatgactccttcttattattctattagtctgtttccatgctcctaataaagacatacccaagactgcaattta

SEQ ID NO: 67 - DNA, 112 bp, Artificial Sequence
  hTMPRSS2 3 prime 112 bp human intergenic sequence immediately 3 prime of TMPRSS2 polyadenylation region
caaaagaaagaagtttattggatttacaattccacatggctggggaggcctcacaatcatggcagaaagcaaggaagagcaaatcacatcttacatggatggcagcaggcag

SEQ ID NO: 68 - DNA, 240 bp, Artificial Sequence
  Tmprss2-gRNA5 Cassette Active genetics cassette comprised of a mouse U6 promoter-driven Tmprss2-gRNA5 guide RNA with 2xPolIII terminator, all in reverse orientation relative to Tmprss2 gene
aaaaaaagtgcctagcaaaaaagcaccgactcggtgccactttttcaagttgataacggactagccttattttaacttgctatttctagctctaaaaccgggtatcagtctgagcacacaaacaaggcttttctccaagggatatttatagtctcaaaacacacaattactttacagttagggtgagtttccttttgtgctgttttttaaaataataatttagtatttgtatctcttata

SEQ ID NO: 69 - DNA, 326 bp, Artificial Sequence; identical to SEQ ID NO: 60
  Splice-Trap FRT Cassette

SEQ ID NO: 70 - DNA, 1352 bp, Artificial Sequence
  Right Homology Arm
tatcagtctgagcacatctgtcctccgagaccaccagtggctcccaatggctacaacttgtatccagcccagtactacccatctccagtgcctcagtatgctccgaggattacaacgcaagcctcaacatctgtcatccacacacatcccaagtcctcaggagcactgtgcacctcaagtaagactccttagcattgtatcacaagggactacagcttctggatctgccttctcttgtggtctcttatttctcctaccttgtcaataacactgcactgccaacagggccaatcttgtcttacctgccttccacattggctggtcagagccatgggctctgcctcatctctctttggcagggagagaccttcctctccccttggcctttggtgcagcaccactgtgtgcttgcccagttcagctggactgttagggcatggtttctcagtcccttcttgcctctgtgcttatggatgtgttgtcttgaatcaacagtaagcactcaaggttgggagtgaactgtcttagatgttattgatgtgctcagaacacagagggctcagggttaagatacctcctgtgactagccagagatctgttggagctctcacctctgccagttcttgccctagaagaagaagggtcttccccacaagggggatttctcccattccagggcctgcccatatactttctaggggagccaggctgcctctccacggcaaaattactctcccagccctggatgtatcatgtcttccccatagccccttaccccccccccccccatagccccagaacagtgctgaaggttgaatatgaaagccgaatctctttgccctctcctgtggccctttcatttgatgattattctcaccattagattccttacctcttatgtccctctaccttttaattctggctgttctcagtgttgcctctctggtgtcaaggacaccagggactctattgataagctactttatatatttattttttaaaactgagcttgtttgtattacatatgtgccagtgcctatggaggccagaaggtgttggatcctctagagctggagtccaaggtattttcggtggttgtaaacactcaacatgaatgctaggaaccaaattcggatcctctggaagagcagtagatgttcctaactgatggctatttctccagctccacaccagctttctagcagagttgctgctttatgagatgagcacctgtctgtgtgtttcatgtatcgttcaaggttgatacaaaagtgaatcctgtgggccacccatacttcatcagcagtcatgtgatagaccttaggcctcaggaatctgtttgtcccacttgg

SEQ ID NO: 71 - DNA, 175 bp, Artificial Sequence
  SA-SV40 poly(A) signal
aatatttttttccctctctagctttacaataattttcccagaacttgtttattgcagcttataatggttacaaataaagcaatagcatcacaaatttcacaaataaagcatttttttcactgcattctagttgtggtttgtccaaactcatcaatgtatcttatcatgtctggat

SEQ ID NO: 72 - DNA, 1945 bp, Artificial Sequence
  FRT-PGK-EM7-Neo-bGHpA-FRT Neomycin resistance gene driven by the mouse PGK promoter and with a bovine growth hormone polyadenylation sequence
gaagttcctattccgaagttcctattctctagaaagtataggaacttcaggtctgaagaggagtttacgtccagccaagctagcttggctgcaggtcgtcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttcgaagttcctattctctagaaagtataggaacttctgcaccatggtatcaccatgcactcaccatgggagattgcgccaacagtattaccactgcctagcgcgtagttaggtgtaatcggctgaactaccagcggccg

SEQ ID NO: 73 - DNA, 1503 bp, Artificial Sequence
  Left Homology Arm
gtcatccaggacaaagtgctaggtgtatcttgttctttcctgctggagaggggaacacatccttagacggtttggttctgggacagacactcacctggtttctcaatctggaatgctctgttaatatgggaatagctttcgctgtcccaccctgaggtttcaagttataatctttggacctttgaaggagagtgctagtgagttattaagcacagatgattaaccttggagtgttggtattgccttaaaattctcaacctttaggccagcaagacagctccataaggtaagagatgacctgcattcaatctttgtggccacagggtggaaggagacaactggctctcacaggttgtcctatgatctctggttacacacacgtgcaacacacacaataaatacatgagcttttaaaagtctgagcatccagtggtccctgctgaggatatctgcttccagaagatgatttttctgtcattcctgaggcccatgagagaacctgagcagaaagagagctggagaggtttggggtaagaaccattagacatgcatagggagacctctctgggagaggtccagctactgagagtagggagagtggaaataacaatgccaacctgtgaacttgctactgaacctgacaactgctgggccatactaggagtacagtctggggaaggtcacgtgaatggactgagtgaaaggattgatacccaagaagtggatcctgccctttttacattcacacagcacttactttgacctccaagttcccctgaaagggcacctacaccctcagcaccaaaatgcatacactctatagggatgttcacactgagctaacaccatcctggtgagaacagatgggtaaaggactaccaactacagctgcatgagagaaggaccctggaagagaatggccacaggagtctgcattgtgggtcccttctagcagtttctcagctcagctgaaaagtgtatttactacacccaaggttgcacacattatctgaatatgaggtccctttagcaggtgagttctcagatatgagctatatcctgaaccttgacattaacctggctcaaaatttcagacctcaaggctcagggggtgatttagaggaggaaccagagaatgcgactctaactgtgtgacttttgactccaaacttgtatgtatgttttctacagaatcttagttatcggtcaccttttctttatttcaaagaacaaataatgaaaaaaaaatggtccaggaaaccaaagctaatgaaacacagatagttgagcgttctagttagtgcagaaacaagagtctagtcatgttaagcaagttgctgtgatgacagagtgattggcaatctgctggctctctccctgttttctgtgaacaatgctaactttccaaacctttagagtgagtgagagctgtaactgatttatctcctttcccattgtttaggggtcacctccaggaatcggaccttgctatgagaaccacgg

SEQ ID NO: 74 - DNA, 1349 bp, Artificial Sequence
  Right Homology Arm
gtatcagtctgagcacatctgtcctccgagaccaccagtggctcccaatggctacaacttgtatccagcccagtactacccatctccagtgcctcagtatgctccgaggattacaacgcaagcctcaacatctgtcatccacacacatcccaagtcctcaggagcaccgtgcacctcaagtaagactccttagcattgtatcacaagggactacagtttctggatctgccttctcttgtggtctcttatttctcctaccttgtcaataacactgcactgccaacagggccaatcttgtcttacctgccttccacattggctggtcagagccatgggctctgcctcatctctctttggcagggagagaccttcctctccccttggcctttggtgcagcaccactgtgtgcttgcccagttcagctggactgttagggcatggtttctcagtcccttcttgcctctgtgcttatggatgtgttgtcttgaatcaacagtaagcactcaaggttgggagtgaactgtcttagatgttattgatgtgctcggaacacggagggctcagggttaagatgcctcctgtgactagccagagatctgttggagctctcacctctgccagttcttgccctagaagaagaagggtcttccccacaagggggatttctcccattccagggcctgctcaaatactttctaggggagccaggctgcctctccatggcaaaattactctcccagccctggatgtatcatgtcttccccatagccccttacccccccccccatagccccagaacagtgctgaaggttgaatatgaaagccaaatctctttgccctctcctgtggccctttcatttgatgattactctcaccattagattccttacctctatgtccctctaccttttaattctggctgttctcagtgttgcttctctggtgtcaaggacaccagggactctattgataagctactttatatatttattttttaaaactgagcttgtgtgtattacatatgtgccagtgcctatggaggccagaaggtgttggatcctctagagctggagtccaaggtattttcggtggttgtaaacactcaacacgaatgctaggaaccaaattcggatcctctggaagagcagtagatgttcctaactgatggctatttctccagctccacaccagctttctagcagaattgctgctttatgagatgagcacctgtctgtgtgtttcatgtgtcgttcaaggttgatacaaaagtgaatcctgtgggccacccatacttcatcagcagtcatgtgatagaccttaggcctcaggaatctgtttgtcccacttgg

SEQ ID NO: 75 - DNA, 110 bp, Artificial Sequence
  Rat ACE2
acggggaaagatgtcaagctcctgctggctccttctcagccttgttgctgttgctactgctcagtccctcatcgaggaaaaggccgagagctttttaaacaagtttaacc

SEQ ID NO: 76 - DNA, 20 bp, Artificial Sequence
  gRNA1 no PAM
gaaagatgtcaagctcctgc

SEQ ID NO: 77 - DNA, 20 bp, Artificial Sequence
  gRNA2 no PAM
tactgctcagtccctcatcg

SEQ ID NO: 78 - DNA, 110 bp, Artificial Sequence
  Rat TMPRSS2
cacaggcaggatggcattgaactcagtaagtggttcatcttacgtctctggccctttccttgttccttcgcccctctggtatcttccacaggtgtccatcagtaatttta

SEQ ID NO: 79 - DNA, 20 bp, Artificial Sequence
  gRNA1 no PAM
aaattactgatggacacctg

SEQ ID NO: 80 - DNA, 20 bp, Artificial Sequence
  gRNA2 no PAM
agtggttcatcttacgtctc

SEQ ID NO: 81 - DNA, 100 bp, Artificial Sequence
  Hamster ACE2
acggggaaagatgtcaagctcctcctggctccttctcagccttgttgctgtaactactgctcagtccatcatcgaggaacaggccaagacatttttagac

SEQ ID NO: 82 - DNA, 20 bp, Artificial Sequence
  gRNA1 no PAM
aacaaggctgagaaggagcc

SEQ ID NO: 83 - DNA, 20 bp, Artificial Sequence
  gRNA2 no PAM
gaaagatgtcaagctcctcc

SEQ ID NO: 84 - DNA, 110 bp, Artificial Sequence
  Hamster TMPRSS2
cacagccaggatggctttgaactcagtaagtggtcgattgttaaatctctggccttctccttgtccattcatccttctgttatcttccatgggtgtccatctgtaatttt

SEQ ID NO: 85 - DNA, 20 bp, Artificial Sequence
  gRNA1 no PAM
atccttctgttatcttccat

SEQ ID NO: 86 - DNA, 20 bp, Artificial Sequence
  gRNA2 no PAM
tactgagttcaaagccatcc

SEQ ID NO: 87 - DNA, 110 bp, Artificial Sequence
  Guinea Pig ACE2
tgttcagtggatgagatctggtggacggggaaagatgtccggctctttctggttcctgctcaatcttgttgctgtaactactgctcagttcaacctcgaggaacaggcca

SEQ ID NO: 88 - DNA, 20 bp, Artificial Sequence
  gRNA1 no PAM
tcagttcaacctcgaggaac

SEQ ID NO: 89 - DNA, 20 bp, Artificial Sequence
  gRNA2 no PAM
tactgctcagttcaacctcg

SEQ ID NO: 90 - DNA, 110 bp, Artificial Sequence
  Guinea Pig TMPRSS2
tgacctcagcatggatttgaactcagtaagtagttaatgattacctccctgccctccccccttccctgcattcccagcagtgcaagggggcagctggaggaggggtaggt

SEQ ID NO: 91 - DNA, 20 bp, Artificial Sequence
  gRNA1 no PAM
ctgagttcaaatccatgctg

SEQ ID NO: 92 - DNA, 20 bp, Artificial Sequence
  gRNA2 no PAM
ccttgcactgctgggaatgc

SEQ ID NO: 93 - DNA, 19 bp, Artificial Sequence
  guide target no PAM
aaggctggaaggagccagg

SEQ ID NO: 94 - DNA, 25 bp, Artificial Sequence
  off-target site 1 contribution
aacctgagaaggctgggaaggagcc

SEQ ID NO: 95 - DNA, 50 bp, Artificial Sequence
  off-target site 1 contribution
aggagggtagagggccaggcccacgggcgcacagagctctgagtttgagt

SEQ ID NO: 96 - DNA, 49 bp, Artificial Sequence
  off-target site 1 contribution
ggagggtagagggccaggcccacgggcgcacagagctctgagtttgagt

SEQ ID NO: 97 - DNA, 20 bp, Artificial Sequence
  off-target site 1
aaggctgggaaggagccagg

SEQ ID NO: 98 - DNA, 20 bp, Artificial Sequence
  off-target site 2
gagactgagaaggagccagg

SEQ ID NO: 99 - DNA, 19 bp, Artificial Sequence
  off-target site 3
gaggtgggaaggagccagg

SEQ ID NO: 100 - DNA, 19 bp, Artificial Sequence
  off-target site 4
agggatggaaggagccagg

SEQ ID NO: 101 - DNA, 19 bp, Artificial Sequence
  off-target site 5
aagatgggaaggagccagg

SEQ ID NO: 102 - DNA, 20 bp, Artificial Sequence
  off-target site 1 Fwd Primer
taccgaaaacctgcaggacc

SEQ ID NO: 103 - DNA, 20 bp, Artificial Sequence
  off-target site 1 Rev Primer
tgttcgctgtatcgacccac

SEQ ID NO: 104 - DNA, 20 bp, Artificial Sequence
  off-target site 2 Fwd Primer
gtggctcagcccttaacaca

SEQ ID NO: 105 - DNA, 20 bp, Artificial Sequence
  off-target site 2 Rev Primer
cgtggtaattgtcatcgcgg

SEQ ID NO: 106 - DNA, 20 bp, Artificial Sequence
  off-target site 3 Fwd Primer
tgggctaaggtcatcaggga

SEQ ID NO: 107 - DNA, 20 bp, Artificial Sequence
  off-target site 3 Rev Primer
cggtgcttgagaactgggta

SEQ ID NO: 108 - DNA, 20 bp, Artificial Sequence
  off-target site 4 Fwd Primer
cacctgtgtggttgggagag

SEQ ID NO: 109 - DNA, 20 bp, Artificial Sequence
  off-target site 4 Rev Primer
ggcttggagacccttctcac

SEQ ID NO: 110 - DNA, 20 bp, Artificial Sequence
  off-target site 5 Fwd Primer
accttggctgtcatcactca

SEQ ID NO: 111 - DNA, 20 bp, Artificial Sequence
  off-target site 5 Rev Primer
gctttgtgcatgtttggcac

SEQ ID NO: 112 - DNA, 20 bp, Artificial Sequence
  off-target site 1
tgtgatcagaatgatacccg

SEQ ID NO: 113 - DNA, 20 bp, Artificial Sequence
  off-target site 2
cgtgatcagaatgatacccg

SEQ ID NO: 114 - DNA, 19 bp, Artificial Sequence
  off-target site 3
tgtctcagactgataccca

SEQ ID NO: 115 - DNA, 20 bp, Artificial Sequence
  off-target site 4
tgtcttctggctgatacccg

SEQ ID NO: 116 - DNA, 21 bp, Artificial Sequence
  off-target site 5
tgtggtcaagactgatacctg

SEQ ID NO: 117 - DNA, 21 bp, Artificial Sequence
  off-target site 1 Fwd Primer
aggtgtagagacgatccagca

SEQ ID NO: 118 - DNA, 21 bp, Artificial Sequence
  off-target site 1 Rev Primer
aggagccatgttcaccatacc

SEQ ID NO: 119 - DNA, 20 bp, Artificial Sequence
  off-target site 2 Fwd Primer
acgctcatggattagcacca

SEQ ID NO: 120 - DNA, 20 bp, Artificial Sequence
  off-target site 2 Rev Primer
acaaccaccccctacatcca

SEQ ID NO: 121 - DNA, 20 bp, Artificial Sequence
  off-target site 3 Fwd Primer
tagctttgctggcaaggtgg

SEQ ID NO: 122 - DNA, 20 bp, Artificial Sequence
  off-target site 3 Rev Primer
tgtgagtggcctcatgatcg

SEQ ID NO: 123 - DNA, 20 bp, Artificial Sequence
  off-target site 4 Fwd Primer
gttgacggtgtccagtgtcc

SEQ ID NO: 124 - DNA, 20 bp, Artificial Sequence
  off-target site 4 Rev Primer
ccctcgtgcatgcctcttac

SEQ ID NO: 125 - DNA, 21 bp, Artificial Sequence
  off-target site 5 Fwd Primer
gttgccttggctgtaactcac

SEQ ID NO: 126 - DNA, 20 bp, Artificial Sequence
  off-target site 5 Rev Primer
tgcttctgaccaggtgcttt

SEQ ID NO: 127 - DNA, 21 bp, Artificial Sequence
  off-target site 1 contribution
aacctgagaaggctgggaagg

SEQ ID NO: 128 - DNA, 36 bp, Artificial Sequence
  off-target site 1 contribution
ccaggcccacgggcgcacagagctctgagtttgagt

SEQ ID NO: 129 - DNA, 22 bp, Artificial Sequence
  off-target site 1 contribution
aacctgagaaggctgggaagga

SEQ ID NO: 130 - DNA, 35 bp, Artificial Sequence
  off-target site 1 contribution
caggcccacgggcgcacagagctctgagtttgagt

SEQ ID NO: 131 - DNA, 23 bp, Artificial Sequence
  off-target site 1 contribution
aacctgagaaggctgggaaggag

SEQ ID NO: 132 - DNA, 38 bp, Artificial Sequence
  off-target site 1 contribution
ggccaggcccacgggcgcacagagctctgagtttgagt